SEQUENCE LISTING (146 sequences)

SEQ ID NO: 1 - DNA, 213 bp, Mycobacterium avium
  source 1..213: mol_type = genomic DNA; sub_species = paratuberculosis
atgaccgtgaccgaggtggtggtggcccagcccgtgtgggccggcgtggacgccggcaaggccgaccactactgcatggtgatcaacgacgacgcccagaggctgctgagccagagggtggccaacgacgaggccgccctgctggagctgatcgccgccgtgaccaccctggccgacggcggcgaggtgacctgggccatcgacctgaacgcc

SEQ ID NO: 2 - protein, 71 aa, Mycobacterium avium
  source 1..71: note = paratuberculosis
MTVTEVVVAQPVWAGVDAGKADHYCMVINDDAQRLLSQRVANDEAALLELIAAVTTLADGGEVTWAIDLNA

SEQ ID NO: 3 - protein, 14 aa, Mycobacterium avium
  source 1..14: note = paratuberculosis
MVINDDAQRLLSQR

SEQ ID NO: 4 - protein, 26 aa, Mycobacterium avium
  source 1..26: note = paratuberculosis
MVINDDAQRLLSQRVANDEAALLELI

SEQ ID NO: 5 - protein, 14 aa, synthetic construct
  REGION 1..14: note = Phosphorylated A0X short peptide
  MOD_RES 12: note = Phosphoserine
MVINDDAQRLLSQR

SEQ ID NO: 6 - protein, 26 aa, synthetic construct
  REGION 1..26: note = Phosphorylated A0X long peptide
  MOD_RES 12: note = Phosphoserine
MVINDDAQRLLSQRVANDEAALLELI

SEQ ID NO: 7 - protein, 15 aa, Mycobacterium avium
  source 1..15: note = paratuberculosis
VTTLADGGEVTWAID

SEQ ID NO: 8 - protein, 18 aa, Mycobacterium avium
  source 1..18: note = paratuberculosis
VTTLADGGEVTWAIDLNA

SEQ ID NO: 9 - protein, 20 aa, Mycobacterium avium
  source 1..20: note = paratuberculosis
EVVVAQPVWAGVDAGKADHY

SEQ ID NO: 10 - protein, 24 aa, Mycobacterium avium
  source 1..24: note = paratuberculosis
MTVTEVVVAQPVWAGVDAGKADHY

SEQ ID NO: 11 - protein, 31 aa, synthetic construct
  REGION 1..29: note = Synthetic Peptide
  REGION 15..16: note = XX = Peptide linker, A, or AA
MVINDDAQRLLSQRXXVTTLADGGEVTWAID

SEQ ID NO: 12 - protein, 34 aa, synthetic construct
  REGION 1..32: note = Synthetic Peptide
  REGION 15..16: note = XX = Peptide linker, A, or AA
MVINDDAQRLLSQRXXVTTLADGGEVTWAIDLNA

SEQ ID NO: 13 - protein, 43 aa, synthetic construct
  REGION 1..41: note = Synthetic Peptide
  REGION 27..28: note = XX = Peptide linker, A, or AA
MVINDDAQRLLSQRVANDEAALLELIXXVTTLADGGEVTWAID

SEQ ID NO: 14 - protein, 46 aa, synthetic construct
  REGION 1..44: note = Synthetic Peptide
  REGION 27..28: note = XX = Peptide linker, A, or AA
MVINDDAQRLLSQRVANDEAALLELIXXVTTLADGGEVTWAIDLNA

SEQ ID NO: 15 - protein, 35 aa, synthetic construct
  REGION 1..34: note = Synthetic Peptide
  VARIANT 21: note = X = Peptide linker or C
EVVVAQPVWAGVDAGKADHYXMVINDDAQRLLSQR

SEQ ID NO: 16 - protein, 47 aa, synthetic construct
  REGION 1..46: note = Synthetic Peptide
  VARIANT 21: note = X = Peptide linker or C
EVVVAQPVWAGVDAGKADHYXMVINDDAQRLLSQRVANDEAALLELI

SEQ ID NO: 17 - protein, 39 aa, synthetic construct
  REGION 1..38: note = Synthetic Peptide
  VARIANT 25: note = X = Peptide linker or C
MTVTEVVVAQPVWAGVDAGKADHYXMVINDDAQRLLSQR

SEQ ID NO: 18 - protein, 51 aa, synthetic construct
  REGION 1..50: note = Synthetic Peptide
  VARIANT 25: note = X = Peptide linker or C
MTVTEVVVAQPVWAGVDAGKADHYXMVINDDAQRLLSQRVANDEAALLELI

SEQ ID NO: 19 - protein, 67 aa, synthetic construct
  REGION 1..64: note = Synthetic Peptide
  VARIANT 21: note = X = Peptide linker or C
  REGION 48..49: note = XX = Peptide linker, A, or AA
EVVVAQPVWAGVDAGKADHYXMVINDDAQRLLSQRVANDEAALLELIXXVTTLADGGEVTWAIDLNA

SEQ ID NO: 20 - protein, 71 aa, synthetic construct
  REGION 1..68: note = Synthetic Peptide
  VARIANT 25: note = X = Peptide linker or C
  REGION 52..53: note = XX = Peptide linker, A, or AA
MTVTEVVVAQPVWAGVDAGKADHYXMVINDDAQRLLSQRVANDEAALLELIXXVTTLADGGEVTWAIDLNA

SEQ ID NO: 21 - DNA, 606 bp, Mycobacterium avium
  source 1..606: mol_type = genomic DNA; sub_species = paratuberculosis
atgcctctgctgaccatcggcgatcagttccccgcctacgagcttgaccgcgtgatcgcgggcgacctgtccaaggtcgacgccaagcagcccggtgactacttcaccaccgtcaccagcgaggaccacgccggcaagtggcgcgtggtgttcttctggcccaaggacttcaccttcgtctgccccaccgagatcgccaccttcggcaagctcaacgacgagttcgaggaccgcgacgcccaggtgctcggcgtctcgatcgacagcgagttcgtccacttcaactggcgcgcccagcacgaggacctgaagaacctgccgttcccgatgctctcggacatcaagcgcgaactgagcctggccaccggtgttctcaacgccgacggcgtggccgaccgggccaccttcatcgtcgacccgaacaacgagatccagttcgtctcggtcaccgcgggttcggtgggccgcaacgtcgaggaagtgctgcgggtgctggatgcgctgcagtccgacgagctgtgcgcgtgcaactggcgcaagggtgacccgacgctgaacgccaccgaactgctcaaggcctctgcttaaaagggcgaattcgtttaa

SEQ ID NO: 22 - protein, 195 aa, Mycobacterium avium
  source 1..195: note = paratuberculosis
MPLLTIGDQFPAYELDRVIAGDLSKVDAKQPGDYFTTVTSEDHAGKWRVVFFWPKDFTFVCPTEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASA

SEQ ID NO: 23 - DNA, 801 bp, Mycobacterium avium
  source 1..801: mol_type = genomic DNA
atgactgcgccagtgttctcgataattatccctaccttcaatgcagcggtgacgctgcaagcctgcctcggaagcatcgtcgggcagacctaccgggaagtggaagtggtccttgtcgacggcggttcgaccgatcggaccctcgacatcgcgaacagtttccgcccggaactcggctcgcgactggtcgttcacagcgggcccgatgatggcccctacgacgccatgaaccgcggcgtcggcgtagccacaggcgaatgggtactttttttaggcgccgacgacaccctctacgaaccaaccacgttggcccaggtagccgcttttctcggcgaccatgcggcaagccatcttgtctatggcgatgttgtgatgcgttcgacgaaaagccggcatgccggacctttcgacctcgaccgcctcctatttgagacgaatttgtgccaccaatcgatcttttaccgccgtgagcttttcgacggcatcggcccttacaacctgcgctaccgagtctgggcggactgggacttcaatattcgctgcttctccaacccggcgctgattacccgctacatggacgtcgtgatttccgaatacaacgacatgaccggcttcagcatgaggcaggggactgataaagagttcagaaaacggctgccaatgtacttctgggttgcagggtgggagacttgcaggcgcatgctggcgtttttgaaagacaaggagaatcgccgtctggccttgcgtacgcggttgataagggttaaggccgtctccaaagaacgaagcgcagaaccgtag

SEQ ID NO: 24 - protein, 266 aa, Mycobacterium avium
  source 1..266: note = paratuberculosis
MTAPVFSIIIPTFNAAVTLQACLGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEP

SEQ ID NO: 25 - DNA, 417 bp, Mycobacterium avium
  source 1..417: mol_type = genomic DNA; sub_species = paratuberculosis
cgatttcgccgccaccgccacgccgaaatcatcctgagcatgcccggattcggcgtcatcctgggcgctgagttcctcgccgccaccggcggggacatggccgcattcgcctccgccgaccgcctcgccggcgtcgccggcctggcgccggtaccacgagattccggccgcatcagcggaaacctcaaacgcccccgacgctacgaccggcgcctgctgcgcgcctgctacctgtcggccttggtcagcatccgcaccgacccctcctcgcgcacctactacgaccgaaaacgcaccgaaggaaaacgccacacccaagccgtcctcgccctggcccgccgccgcctcaacgtcctgtgggccatgctgcgcgaccacgctgtctaccaccccgcaaccactaccgcggcggcttga

SEQ ID NO: 26 - protein, 138 aa, Mycobacterium avium
RFRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAA

SEQ ID NO: 27 - DNA, 402 bp, Mycobacterium avium
  source 1..402: mol_type = genomic DNA; sub_species = paratuberculosis
cgatttcgccgccaccgccacgccgaaatcatcctgagcatgcccggattcggcgtcatcctgggcgctgagttcctcgccgccaccggcggggacatggccgcattcgcctccgccgaccgcctcgccggcgtcgccggcctggcgccggtaccacgagattccggccgcatcagcggaaacctcaaacgcccccgacgctacgaccggcgcctgctgcgcgcctgcgtcagcatccgcaccgacccctcctcgcgcacctactacgaccgaaaacgcaccgaaggaaaacgccacacccaagccgtcctcgccctggcccgccgccgcctcaacgtcctgtgggccatgctgcgcgaccacgctgtctaccaccccgcaaccactaccgcggcggcttga

SEQ ID NO: 28 - protein, 133 aa, Mycobacterium avium
  source 1..133: note = paratuberculosis
RFRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAA

SEQ ID NO: 29 - DNA, 1335 bp, Mycobacterium avium
  source 1..1335: mol_type = genomic DNA; sub_species = paratuberculosis
gtgactgaagccaatgagtgcaactcggcgtcgcgaaaggtttcagtcgcggttgagcaagacaccgcaagactactggagtgcgtgcacaagcgcccccagctcgcggctgaaagcggatgcaaaggggttcgaagcttgagcaacatgcgaaggggagaacggcctatgagcctgggacaggttttcgacccgcgcgcgaatgcacttaatgcgtggcgcttggtgttggcgagcggggtgatcctatggcattcgtttccgctcactggacgtatgccgtgggcgccgttcgtccagttgcttggccttggatgcgttgatggtttctttgcggtctccggctatctcatcgtctcgagctggcttcgcaacccgcatcccgcccaatacttcaccgctcgatgtcttcgtattctcccgggtctgtggatctgtctcatcttgacggcgtttgtcatcgctccgataggtgtgggcgcccagggcggttcggccgcgaaactactgatgtccggcgctccgatcgagtatgtgctaaaagacagtgcggtttggatggttaagttcgatatcggtggcacacctcgcgatattccagttgcgggtatttggaacggttctctgtggacattgggttgggaggtgctttgctatatcggcgtagcagtatttggtatgctcggacttcttagtcgccgttggttcgttccagggatattgatcctggcgctgtcgtggtcggtgttcttgccggcatggggcggaatacacgcgatcgcctccaatgctgcgcgattcgctgtgatgttttcggccggagcgttgctgtatcaattccgtaacgtgattccggctcggtggtccttcgttgccgtcggcctcattatcgttgtggtttcctctgccgtgctgccggactaccggttggtggcggcccttccgatggcgtacctaatcatcgcttcgggttcgctcatccacaatcaaaggatgaggttccgcaccgatctatcctatggagtatatatttatgcgtttccaattcagcaagtgctggtcctgtgtggattcgccgagataaatccaatcgctttctgcgcgatttctgtcgcagctattttgccgctcgccgcgctcagttggttcttggtcgagaaacctgcgttgtcctggaagagtcgtctccggcggaaaaacagttcaattgcgctagccaatatggaagatggtggatcagtcggccgctcaaatgacattcccggaaggcgggcccgctttattggcgagaaagccgaagatcctcccgcgccgagcccaagaccggctttgtaa

SEQ ID NO: 30 - protein, 444 aa, Mycobacterium avium
  source 1..444: note = paratuberculosis
VTEANECNSASRKVSVAVEQDTARLLECVHKRPQLAAESGCKGVRSLSNMRRGERPMSLGQVFDPRANALNAWRLVLASGVILWHSFPLTGRMPWAPFVQLLGLGCVDGFFAVSGYLIVSSWLRNPHPAQYFTARCLRILPGLWICLILTAFVIAPIGVGAQGGSAAKLLMSGAPIEYVLKDSAVWMVKFDIGGTPRDIPVAGIWNGSLWTLGWEVLCYIGVAVFGMLGLLSRRWFVPGILILALSWSVFLPAWGGIHAIASNAARFAVMFSAGALLYQFRNVIPARWSFVAVGLIIVVVSSAVLPDYRLVAALPMAYLIIASGSLIHNQRMRFRTDLSYGVYIYAFPIQQVLVLCGFAEINPIAFCAISVAAILPLAALSWFLVEKPALSWKSRLRRKNSSIALANMEDGGSVGRSNDIPGRRARFIGEKAEDPPAPSPRPAL

SEQ ID NO: 31 - DNA, 585 bp, synthetic construct
  misc_feature 1..585: note = Codon Optimised ahpC
  source 1..585: mol_type = other DNA
ccccttctcactatcggagaccagttccccgcttacgaacttacagctcttatcgctggagatctgagtaaggttgacgccaaacagcccggcgattatttcactaccgttaccagtgaggatcacgcaggtaaatggagagtcgtcttcttctggcctaaagacttcacctttgtgtgccctactgagatcgcaacattcgggaagctgaacgatgagttcgaagatcgagacgcacaggttttgggcgtgtctatcgattccgagttcgtgcacttcaactggagagcacagcatgaagatctcaagaaccttccattccccatgctcagcgacatcaagagagaactgagcttggcaacaggtgttctgaatgctgatggcgttgctgacagagcaacattcattgttgaccccaataacgagatccagttcgtttccgttactgctggttctgtcggtagaaacgttgaagaggtcctgagagttctcgacgcacttcagagtgatgaactgtgtgcctgcaattggcggaaaggagatcctactctcaatgccacagagctgcttaaagcaagtgctctc

SEQ ID NO: 32 - protein, 195 aa, Mycobacterium avium
  source 1..195: note = paratuberculosis
PLLTIGDQFPAYELTALIAGDLSKVDAKQPGDYFTTVTSEDHAGKWRVVFFWPKDFTFVCPTEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASAL

SEQ ID NO: 33 - DNA, 729 bp, synthetic construct
  misc_feature 1..729: note = Modified Gsd
  source 1..729: mol_type = other DNA
ggatccattgtcggacagacctatagagaggtggaagttgtcctggtcgatggtggatctacagataggactctcgacattgccaactcctttagaccagagctcggttcaaggctcgttgttcattctggaccagatgatggaccatacgacgccatgaacagaggtgttggagttgctacaggagaatgggtcttgttccttggagctgatgacactctgtacgaaccgactacattggctcaggttgcagcatttttgggagatcatgcagcttctcaccttgtgtacggagatgtggtcatgagatccaccaagtccagacatgctggaccattcgatcttgacagactcctgttcgagaccaacctctgtcatcagagcatcttctacagacgggaactcttcgacggaattggaccttacaacctcaggtacagggtttgggcagactgggatttcaacatcaggtgcttctcgaacccagctttgatcacacggtacatggatgttgtgatctccgagtacaacgatatgaccggcttctccatgagacagggaaccgacaaagagttcaggaagcgcttgccaatgtacttctgggttgctggatgggaaacatgtcggagaatgcttgctttcctgaaggacaaggagaacaggagacttgctctcaggactagactcatcagggtcaaagcagtgtccaaggaaaggagtgctgaacct

SEQ ID NO: 34 - protein, 243 aa, Mycobacterium avium
  source 1..243: note = paratuberculosis
GSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEP

SEQ ID NO: 35 - DNA, 420 bp, synthetic construct
  misc_feature 1..420: note = Codon Optimised P12
  source 1..420: mol_type = other DNA
agaattcggagacatagacatgcagagatcatcctgagcatgcctggatttggcgttatcctcggagctgaatttcttgcagcaacaggaggtgatatggcagctttcgcatcagctgacagattggctggagttgcaggtttggctccagttccaagagattcagggagaatcagcggtaacctcaagagacctagacgctacgacagaagactgcttagagcctgctatctgagtgctttggttagcattagaaccgacccctctagtcgaacctactacgataggaagcggactgaaggtaagagacatacccaggcagtgttggcacttgctagaagacggcttaatgttctgtgggctatgctgagagatcatgccgtgtaccatcctgctaccacaacagctgctgctagactt

SEQ ID NO: 36 - protein, 140 aa, Mycobacterium avium
  source 1..140: note = paratuberculosis
RIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARL

SEQ ID NO: 37 - DNA, 405 bp, synthetic construct
  misc_feature 1..405: note = Modified P12
  source 1..405: mol_type = other DNA
agaattcggagacatagacatgcagagatcatcctgagcatgcctggatttggcgttatcctcggagctgaatttcttgcagcaacaggaggtgatatggcagctttcgcatcagctgacagattggctggagttgcaggtttggctccagttccaagagattcagggagaatcagcggtaacctcaagagacctagacgctacgacagaagactgcttagagcctgcgttagcattagaaccgacccctctagtcgaacctactacgataggaagcggactgaaggtaagagacatacccaggcagtgttggcacttgctagaagacggcttaatgttctgtgggctatgctgagagatcatgccgtgtaccatcctgctaccacaacagctgctgctagactt

SEQ ID NO: 38 - protein, 135 aa, Mycobacterium avium
  source 1..135: note = paratuberculosis
RIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARL

SEQ ID NO: 39 - DNA, 738 bp, synthetic construct
  misc_feature 1..738: note = Modified Mpa
  source 1..738: mol_type = other DNA
aagcttcgcagaggtgagagacctatgagtcttggccaggtctttgatcctagagctaatgcactgcactctttccctcttacaggacgcatgccttgggctccatttatcgttagttcctggctcagaaaccctcatccagctcagtacttcacagccagatgtctcagaatccttcctggtctttggattggagcacagggtggttccgcagctaagctgttgatgagtggtgcaccaatcgaatacgtcctgaaagactcagcagtgtggatgttcaagttcgacattggaggaacaccaagggatattcctgtcgctggtatctggaatggaagtttgtggaccccagcatggggaggtattcatgctatcgcttccaacgcttaccagttccgaaatgtgatccctgcaagatggtctgtgagttcagccgtgttgccaaactatagacttgttgctgctctccccatggcctaccataatcagcgaatgaggtttcggacagatctgtcctatggtgtgtacgggttcgctgaaatcaatcccatcgctctggttgagaaacctgccctgtcttggaaatccagactgagacggaagaactcttccatcgctctcgcaaacatggaagatggtggtagtgttggaaggagtaacgacatccctgggaggagggctagatttattggtgagaaagccgaagatcctcctgctccatctcctagacccgccttg

SEQ ID NO: 40 - protein, 246 aa, Mycobacterium avium
  source 1..246: note = paratuberculosis
KLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPAL

SEQ ID NO: 41 - protein, 833 aa, synthetic construct
  REGION 1..833: note = vaccine polypeptide
MQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 42 - protein, 838 aa, synthetic construct
  REGION 1..838: note = vaccine polypeptide
MQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 43 - protein, 406 aa, Mycobacterium avium
  source 1..406: note = paratuberculosis
MTVTEVVVAQPVWAGVDAGKADHYCMVINDDAQRLLSQRVANDEAALLELIAAVTTLADGGEVTWAIDLNAGGAALLIALLIAAGQRLLYIPGRTVHHAAGSYRGEGKTDAKDAAIIADQARMRHDLQPLRAGDDIAVELRILTSRRSDLVADRTRAINRMRAQLLEYFPALERAFDYNKSRAALILLTGYQTPDALRSAGGARVAAFLRKRKARNADTVAATALQAANAQHSIVPGQQLAATVVARLAKEVMALDTEIGDTDAMIEERFRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAA

SEQ ID NO: 44 - protein, 10 aa, Mycobacterium avium
  source 1..10: note = paratuberculosis
VSIRTDPSSR

SEQ ID NO: 45 - protein, 10 aa, synthetic construct
  REGION 1..10: note = Phosphorylated A4 peptide
  MOD_RES 8: note = Phosphoserine
VSIRTDPSSR

SEQ ID NO: 46 - protein, 10 aa, synthetic construct
  REGION 1..10: note = Phosphorylated A4 peptide
  MOD_RES 9: note = Phosphoserine
VSIRTDPSSR

SEQ ID NO: 47 - protein, 10 aa, synthetic construct
  REGION 1..10: note = Phosphorylated A4 peptide
  MOD_RES 8: note = Phosphoserine
  MOD_RES 9: note = Phosphoserine
VSIRTDPSSR

SEQ ID NO: 48 - protein, 10 aa, Mycobacterium avium
  source 1..10: note = paratuberculosis
NLKRPRRYDR

SEQ ID NO: 49 - DNA, 2522 bp, synthetic construct
  misc_feature 1..2522: note = vaccine polynucleotide sequence
  source 1..2522: mol_type = other DNA
atgcagatcttcgtcaaactgccccttctcactatcggagaccagttccccgcttacgaacttacagctcttatcgctggagatctgagtaaggttgacgccaaacagcccggcgattatttcactaccgttaccagtgaggatcacgcaggtaaatggagagtcgtcttcttctggcctaaagacttcacctttgtgtgccctactgagatcgcaacattcgggaagctgaacgatgagttcgaagatcgagacgcacaggttttgggcgtgtctatcgattccgagttcgtgcacttcaactggagagcacagcatgaagatctcaagaaccttccattccccatgctcagcgacatcaagagagaactgagcttggcaacaggtgttctgaatgctgatggcgttgctgacagagcaacattcattgttgaccccaataacgagatccagttcgtttccgttactgctggttctgtcggtagaaacgttgaagaggtcctgagagttctcgacgcacttcagagtgatgaactgtgtgcctgcaattggcggaaaggagatcctactctcaatgccacagagctgcttaaagcaagtgctctcggatccattgtcggacagacctatagagaggtggaagttgtcctggtcgatggtggatctacagataggactctcgacattgccaactcctttagaccagagctcggttcaaggctcgttgttcattctggaccagatgatggaccatacgacgccatgaacagaggtgttggagttgctacaggagaatgggtcttgttccttggagctgatgacactctgtacgaaccgactacattggctcaggttgcagcatttttgggagatcatgcagcttctcaccttgtgtacggagatgtggtcatgagatccaccaagtccagacatgctggaccattcgatcttgacagactcctgttcgagaccaacctctgtcatcagagcatcttctacagacgggaactcttcgacggaattggaccttacaacctcaggtacagggtttgggcagactgggatttcaacatcaggtgcttctcgaacccagctttgatcacacggtacatggatgttgtgatctccgagtacaacgatatgaccggcttctccatgagacagggaaccgacaaagagttcaggaagcgcttgccaatgtacttctgggttgctggatgggaaacatgtcggagaatgcttgctttcctgaaggacaaggagaacaggagacttgctctcaggactagactcatcagggtcaaagcagtgtccaaggaaaggagtgctgaacctagaattcggagacatagacatgcagagatcatcctgagcatgcctggatttggcgttatcctcggagctgaatttcttgcagcaacaggaggtgatatggcagctttcgcatcagctgacagattggctggagttgcaggtttggctccagttccaagagattcagggagaatcagcggtaacctcaagagacctagacgctacgacagaagactgcttagagcctgctatctgagtgctttggttagcattagaaccgacccctctagtcgaacctactacgataggaagcggactgaaggtaagagacatacccaggcagtgttggcacttgctagaagacggcttaatgttctgtgggctatgctgagagatcatgccgtgtaccatcctgctaccacaacagctgctgctagacttaagcttcgcagaggtgagagacctatgagtcttggccaggtctttgatcctagagctaatgcactgcactctttccctcttacaggacgcatgccttgggctccatttatcgttagttcctggctcagaaaccctcatccagctcagtacttcacagccagatgtctcagaatccttcctggtctttggattggagcacagggtggttccgcagctaagctgttgatgagtggtgcaccaatcgaatacgtcctgaaagactcagcagtgtggatgttcaagttcgacattggaggaacaccaagggatattcctgtcgctggtatctggaatggaagtttgtggaccccagcatggggaggtattcatgctatcgcttccaacgcttaccagttccgaaatgtgatccctgcaagatggtctgtgagttcagccgtgttgccaaactatagacttgttgctgctctccccatggcctaccataatcagcgaatgaggtttcggacagatctgtcctatggtgtgtacgggttcgctgaaatcaatcccatcgctctggttgagaaacctgccctgtcttggaaatccagactgagacggaagaactcttccatcgctctcgcaaacatggaagatggtggtagtgttggaaggagtaacgacatccctgggaggagggctagatttattggtgagaaagccgaagatcctcctgctccatctcctagacccgccttgaggattccaaaccctcttctcggtcttga

SEQ ID NO: 50 - protein, 9 aa, synthetic construct
  REGION 1..9: note = Synthetic Peptide
VDAGKADHY

SEQ ID NO: 51 - protein, 68 aa, synthetic construct
  REGION 1..68: note = Synthetic polypeptide
MTVTEVVVAQPVWAGVDAGKADHYCMVINDDAQRLLSQRVANDEAALLELIAAVTTLADGGEVTWAID

SEQ ID NO: 52 - protein, 39 aa, synthetic construct
  REGION 1..39: note = Synthetic polypeptide
MVINDDAQRLLSQRVDAGKADHYAVTTLADGGEVTWAID

SEQ ID NO: 53 - protein, 22 aa, synthetic construct
  REGION 1..22: note = Synthetic polypeptide
LNAGGAALLIALLIAAGQRLLY

SEQ ID NO: 54 - protein, 16 aa, synthetic construct
  REGION 1..16: note = Synthetic peptide
IPGRTVHHAAGSYRGE

SEQ ID NO: 55 - protein, 16 aa, synthetic construct
  REGION 1..16: note = Synthetic peptide
IPGATVHHAAGSYRGE

SEQ ID NO: 56 - protein, 38 aa, synthetic construct
  REGION 1..38: note = Synthetic polypeptide
LNAGGAALLIALLIAAGQRLLYIPGRTVHHAAGSYRGE

SEQ ID NO: 57 - protein, 38 aa, synthetic construct
  REGION 1..38: note = Synthetic polypeptide
LNAGGAALLIALLIAAGQRLLYIPGATVHHAAGSYRGE

SEQ ID NO: 58 - protein, 106 aa, synthetic construct
  REGION 1..106: note = Synthetic polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGRTVHHAAGSYRGE

SEQ ID NO: 59 - protein, 106 aa, synthetic construct
  REGION 1..106: note = Synthetic polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGATVHHAAGSYRGE

SEQ ID NO: 60 - protein, 77 aa, synthetic construct
  REGION 1..77: note = Synthetic polypeptide
MVINDDAQRLLSQRVDAGKADHYAVTTLADGGEVTWAIDLNAGGAALLIALLIAAGQRLLYIPGRTVHHAAGSYRGE

SEQ ID NO: 61 - protein, 77 aa, synthetic construct
  REGION 1..77: note = Synthetic polypeptide
MVINDDAQRLLSQRVDAGKADHYAVTTLADGGEVTWAIDLNAGGAALLIALLIAAGQRLLYIPGATVHHAAGSYRGE

SEQ ID NO: 62 - protein, 968 aa, synthetic construct
  REGION 1..968: note = Vaccine polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGATVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 63 - protein, 963 aa, synthetic construct
  REGION 1..963: note = Vaccine polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGATVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 64 - protein, 968 aa, synthetic construct
  REGION 1..968: note = Vaccine polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGRTVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 65 - protein, 963 aa, synthetic construct
  REGION 1..963: note = Vaccine polypeptide
MTVTEVVVAQPVWAGVDAGKADHYCMVINDDAQRLLSQRVANDEAALLELIAAVTTLADGGEVTWAIDLNAGGAALLIALLIAAGQRLLYIPGRTVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 66 - protein, 939 aa, synthetic construct
  REGION 1..939: note = Vaccine polypeptide
MVINDDAQRLLSQRVDAGKADHYAVTTLADGGEVTWAIDLNAGGAALLIALLIAAGQRLLYIPGATVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 67 - protein, 934 aa, synthetic construct
  REGION 1..934: note = Vaccine polypeptide
MVINDDAQRLLSQRVDAGKADHYAVTTLADGGEVTWAIDLNAGGAALLIALLIAAGQRLLYIPGATVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 68 - protein, 939 aa, synthetic construct
  REGION 1..939: note = Vaccine polypeptide
MVINDDAQRLLSQRVDAGKADHYAVTTLADGGEVTWAIDLNAGGAALLIALLIAAGQRLLYIPGRTVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACYLSALVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 69 - protein, 934 aa, synthetic construct
  REGION 1..934: note = Vaccine polypeptide
MVINDDAQRLLSQRVDAGKADHYAVTTLADGGEVTWAIDLNAGGAALLIALLIAAGQRLLYIPGRTVHHAAGSYRGEAPVKQTLNFDLLKLAGDVESNPGPMQIFVKLPLLTIGDQFPAYELTALIAGDLSKVDAKQPGDYFTTVTSEDHAGKWRVVFFWPKDFTGPEIATFGKLNDEFEDRDAQVLGVSIDSEFVHFNWRAQHEDLKNLPFPMLSDIKRELSLATGVLNADGVADRATFIVDPNNEIQFVSVTAGSVGRNVEEVLRVLDALQSDELCACNWRKGDPTLNATELLKASALGSIVGQTYREVEVVLVDGGSTDRTLDIANSFRPELGSRLVVHSGPDDGPYDAMNRGVGVATGEWVLFLGADDTLYEPTTLAQVAAFLGDHAASHLVYGDVVMRSTKSRHAGPFDLDRLLFETNLCHQSIFYRRELFDGIGPYNLRYRVWADWDFNIRCFSNPALITRYMDVVISEYNDMTGFSMRQGTDKEFRKRLPMYFWVAGWETCRRMLAFLKDKENRRLALRTRLIRVKAVSKERSAEPRIRRHRHAEIILSMPGFGVILGAEFLAATGGDMAAFASADRLAGVAGLAPVPRDSGRISGNLKRPRRYDRRLLRACVSIRTDPSSRTYYDRKRTEGKRHTQAVLALARRRLNVLWAMLRDHAVYHPATTTAAARLKLRRGERPMSLGQVFDPRANALHSFPLTGRMPWAPFIVSSWLRNPHPAQYFTARCLRILPGLWIGAQGGSAAKLLMSGAPIEYVLKDSAVWMFKFDIGGTPRDIPVAGIWNGSLWTPAWGGIHAIASNAYQFRNVIPARWSVSSAVLPNYRLVAALPMAYHNQRMRFRTDLSYGVYGFAEINPIALVEKPALSWKSRLRRKNSSIALANMEDGGSVGRSNDIPGRRARFIGEKAEDPPAPSPRPALRIPNPLLGLD

SEQ ID NO: 70 - protein, 9 aa, synthetic construct
  REGION 1..9: note = a 2A peptide fragment
  SITE 5: note = X = A or R or N or D or C or Q or E or G or H or I or L or K or M or F or P or S or T or W or Y or V
GDVEXNPGP

SEQ ID NO: 71 - protein, 24 aa, synthetic construct
  REGION 1..24: note = a 2A peptide
APVKQTLNFDLLKLAGDVESNPGP

SEQ ID NO: 72 - protein, 4 aa, synthetic construct
  REGION 1..4: note = peptide fragment
MTVT

SEQ ID NO: 73 - protein, 10 aa, synthetic construct
  REGION 1..10: note = Synthetic peptide
VDAGKADHYA

SEQ ID NO: 74 - protein, 7 aa, synthetic construct
  REGION 1..7: note = leader sequence
MQIFVKL

SEQ ID NO: 75 - protein, 13 aa, synthetic construct
  REGION 1..13: note = peptide tag
PALRIPNPLLGLD

SEQ ID NO: 76 - protein, 9 aa, synthetic construct
  REGION 1..9: note = Mpa T cell epitope
GFAEINPIA

SEQ ID NO: 77 - protein, 4 aa, synthetic construct
  REGION 1..4: note = peptide linker
SGSG

SEQ ID NO: 78 - protein, 4 aa, synthetic construct
  REGION 1..4: note = peptide linker
GGGS

SEQ ID NO: 79 - protein, 5 aa, synthetic construct
  REGION 1..5: note = peptide linker
EAAAK

SEQ ID NO: 80 - protein, 12 aa, synthetic construct
  REGION 1..12: note = peptide linker
HEYGAEALERAG

SEQ ID NO: 81 - protein, 15 aa, synthetic construct
  REGION 1..15: note = Synthetic peptide
YLSALVSIRTDPSSR

SEQ ID NO: 82 - protein, 15 aa, synthetic construct
  REGION 1..15: note = Synthetic peptide
  MOD_RES 14: note = Phosphoserine
YLSALVSIRTDPSSR

SEQ ID NO: 83 - protein, 106 aa, synthetic construct; identical to SEQ ID NO: 59
  REGION 1..106: note = vaccine polypeptide

SEQ ID NO: 84 - protein, 77 aa, synthetic construct; identical to SEQ ID NO: 61
  REGION 1..77: note = vaccine polypeptide

SEQ ID NO: 85 - protein, 15 aa, Mycobacterium avium
  source 1..15: note = paratuberculosis
ELIAAVTTLADGGEV

SEQ ID NO: 86 - protein, 15 aa, Mycobacterium avium
  source 1..15: note = paratuberculosis
DRKRTEGKRHTQAVL

SEQ ID NO: 87 - protein, 15 aa, Mycobacterium avium
  source 1..15: note = paratuberculosis
NKSRAALILLTGYQT

SEQ ID NO: 88 - protein, 15 aa, Mycobacterium avium
  source 1..15: note = paratuberculosis
AKEVMALDTEIGDTD

SEQ ID NO: 89 - protein, 15 aa, Mycobacterium avium
  source 1..15: note = paratuberculosis
ALDTEIGDTDAMIEE

SEQ ID NO: 90 - protein, 40 aa, Mycobacterium avium
  source 1..40: note = paratuberculosis
GRISGNLKRPRRYDRRLLRACYLSALVSIRTDPSSRTYYD

SEQ ID NO: 91 - protein, 18 aa, Mycobacterium avium
  source 1..18: note = paratuberculosis
NKSRAALILLTGYQTPDA

SEQ ID NO: 92 - protein, 18 aa, Mycobacterium avium
  source 1..18: note = paratuberculosis
NLKRPRRYDRRLLRAGYL

SEQ ID NO: 93 - protein, 10 aa, Mycobacterium avium
  source 1..10: note = paratuberculosis
MVINDDAQRL

SEQ ID NO: 94 - protein, 11 aa, Mycobacterium avium
  source 1..11: note = paratuberculosis
MVINDDAQRLC

SEQ ID NO: 95 - protein, 11 aa, Mycobacterium avium
  source 1..11: note = paratuberculosis
CNLKRPRRYDR

SEQ ID NO: 96 - protein, 11 aa, Mycobacterium avium
  source 1..11: note = paratuberculosis
CVSIRTDPSSR

SEQ ID NO: 97 - protein, 15 aa, Mycobacterium avium
  source 1..15: note = paratuberculosis
MVINDDAQRLLSQRC

SEQ ID NO: 98 - protein, 16 aa, Mycobacterium avium
  source 1..16: note = paratuberculosis
VTTLADGGEVTWAIDC

SEQ ID NO: 99 - protein, 16 aa, Mycobacterium avium
  source 1..16: note = paratuberculosis
YLSALVSIRTDPSSRC

SEQ ID NO: 100 - protein, 10 aa, Mycobacterium avium
  MOD_RES 1: note = acetylated methionine
  SITE 10: note = 8branchedPolylysineOctamer
  source 1..10: note = paratuberculosis
MVINDDAQRL

SEQ ID NO: 101 - protein, 10 aa, Mycobacterium avium
  SITE 1: note = Biotinyl
  MOD_RES 10: note = amidated leucine
  source 1..10: note = paratuberculosis
MVINDDAQRL

SEQ ID NO: 102 - protein, 9 aa, Mycobacterium avium
  SITE 1: note = Biotinyl
  MOD_RES 9: note = amidated arginine
  source 1..9: note = paratuberculosis
MVINDDLQR

SEQ ID NO: 103 - protein, 8 aa, Mycobacterium avium
  SITE 1: note = Biotinyl
  MOD_RES 8: note = amidated glutamic acid
  source 1..8: note = paratuberculosis
MVINNDAE

SEQ ID NO: 104 - protein, 11 aa, Mycobacterium avium
  MOD_RES 1: note = acetylated valine
  SITE 11: note = 8branchedPolylysineOctamer
  source 1..11: note = paratuberculosis
VTTLADGGEVT

SEQ ID NO: 105 - protein, 11 aa, Mycobacterium avium
  SITE 1: note = Biotinyl
  MOD_RES 11: note = amidated threonine
  source 1..11: note = paratuberculosis
VTTLADGGEVT

SEQ ID NO: 106 - protein, 11 aa, Mycobacterium avium
  SITE 1: note = Biotinyl
  MOD_RES 11: note = amidated threonine
  source 1..11: note = paratuberculosis
VATMADGGEVT

SEQ ID NO: 107 - protein, 11 aa, Mycobacterium avium
  SITE 1: note = Biotinyl
  MOD_RES 11: note = amidated threonine
  source 1..11: note = paratuberculosis
VTRLADGGEVT

SEQ ID NO: 108 - protein, 10 aa, Mycobacterium avium
  MOD_RES 1: note = acetylated asparagine
  SITE 10: note = 8branchedPolylysineOctamer
  source 1..10: note = paratuberculosis
NLKRPRRYDR

SEQ ID NO: 109 - protein, 10 aa, Mycobacterium avium
  SITE 1: note = Biotinyl
  MOD_RES 10: note = amidated arginine
  source 1..10: note = paratuberculosis
NLKRPRRYDR

SEQ ID NO: 110 - protein, 7 aa, Mycobacterium avium
  SITE 1: note = Biotinyl
  MOD_RES 7: note = amidated arginine
  source 1..7: note = paratuberculosis
NLKRPRR

SEQ ID NO: 111 - protein, 10 aa, Mycobacterium avium
  SITE 1: note = Biotinyl
  MOD_RES 10: note = acetylated arginine
  source 1..10: note = paratuberculosis
NLRRPRRYHR

SEQ ID NO: 112 - protein, 10 aa, Mycobacterium avium
  SITE 1: note = Biotinyl
  MOD_RES 10: note = amidated arginine
  source 1..10: note = paratuberculosis
NLHRPRRYHR

SEQ ID NO: 113 - protein, 10 aa, Mycobacterium avium
  SITE 1: note = Biotinyl
  MOD_RES 10: note = amidated arginine
  source 1..10: note = paratuberculosis
NMRRPRRYNR

SEQ ID NO: 114 - protein, 10 aa, Mycobacterium avium
  SITE 1: note = Biotinyl
  MOD_RES 10: note = amidated arginine
  source 1..10: note = paratuberculosis
NLRRPKRYNR

SEQ ID NO: 115 - protein, 10 aa, Mycobacterium avium
  SITE 1: note = Biotinyl
  MOD_RES 10: note = amidated arginine
  source 1..10: note = paratuberculosis
NLQRPRRYNR

SEQ ID NO: 116 - protein, 10 aa, Mycobacterium avium
  MOD_RES 1: note = acetylated valine
  SITE 10: note = 8branchedPolylysineOctamer
  source 1..10: note = paratuberculosis
VSIRTDPSSR

SEQ ID NO: 117 - protein, 10 aa, Mycobacterium avium
  SITE 1: note = Biotinyl
  MOD_RES 10: note = amidated arginine
  source 1..10: note = paratuberculosis
VSIRTDPSSR

SEQ ID NO: 118 - protein, 7 aa, Mycobacterium avium
  SITE 1: note = biotinyl
  MOD_RES 7: note = amidated proline
  source 1..7: note = paratuberculosis
VSIRTDP

SEQ ID NO: 119 - protein, 9 aa, Mycobacterium avium
  SITE 1: note = Biotinyl
  MOD_RES 9: note = amidated arginine
  source 1..9: note = paratuberculosis
SIRSDPSSR

SEQ ID NO: 120 - protein, 10 aa, Mycobacterium avium
  SITE 1: note = Biotinyl
  MOD_RES 10: note = amidated arginine
  source 1..10: note = paratuberculosis
YSIRSDPASR

SEQ ID NO: 121 - protein, 10 aa, Mycobacterium avium
  SITE 1: note = Biotinyl
  MOD_RES 10: note = amidated arginine
  source 1..10: note = paratuberculosis
VSVRYDPSSR

SEQ ID NO: 122 - protein, 10 aa, Mycobacterium avium
  SITE 1: note = Biotinyl
  MOD_RES 10: note = amidated arginine
  source 1..10: note = paratuberculosis
IAIRTDPASR

SEQ ID NO: 123 - protein, 11 aa, Mycobacterium avium
  MOD_RES 1: note = acetylated methionine
  source 1..11: note = paratuberculosis
MVINDDAQRLC

SEQ ID NO: 124 - protein, 12 aa, Mycobacterium avium
  MOD_RES 1: note = acetylated valine
  source 1..12: note = paratuberculosis
VTTLADGGEVTC

SEQ ID NO: 125 - protein, 11 aa, Mycobacterium avium
  MOD_RES 1: note = acetylated asparagine
  source 1..11: note = paratuberculosis
NLKRPRRYDRC

SEQ ID NO: 126 - protein, 11 aa, Mycobacterium avium
  MOD_RES 1: note = acetylated valine
  source 1..11: note = paratuberculosis
VSIRTDPSSRC

SEQ ID NO: 127 - protein, 15 aa, Mycobacterium avium
  MOD_RES 15: note = amidated arginine
  source 1..15: note = paratuberculosis
CMVINDDAQRLLSQR

SEQ ID NO: 128 - protein, 14 aa, Mycobacterium avium
  SITE 1: note = Biotinyl
  MOD_RES 14: note = amidated arginine
  source 1..14: note = paratuberculosis
MVINDDAQRLLSQR

SEQ ID NO: 129 - protein, 14 aa, Mycobacterium avium
  SITE 1: note = Biotinyl
  MOD_RES 14: note = amidated leucine
  source 1..14: note = paratuberculosis
MVINDDLQRIILFL

SEQ ID NO: 130 - protein, 14 aa, Mycobacterium avium
  SITE 1: note = Biotinyl
  MOD_RES 14: note = amidated leucine
  source 1..14: note = paratuberculosis
MSINDDAQKLKDRL

SEQ ID NO: 131 - protein, 15 aa, Mycobacterium avium
  MOD_RES 13: note = Phosphoserine
  SITE 15: note = Amide BSA conjugated
  source 1..15: note = paratuberculosis
CMVINDDAQRLLSQR

SEQ ID NO: 132 - protein, 14 aa, Mycobacterium avium
  SITE 1: note = Biotinyl
  MOD_RES 12: note = Phosphoserine
  MOD_RES 14: note = amidated arginine
  source 1..14: note = paratuberculosis
MVINDDAQRLLSQR

SEQ ID NO: 133 - protein, 14 aa, Mycobacterium avium
  SITE 1: note = Biotinyl
  MOD_RES 14: note = amidated arginine
  source 1..14: note = paratuberculosis
MVINDDAQRLLSQR

SEQ ID NO: 134 - protein, 21 aa, Mycobacterium avium
  MOD_RES 1: note = acetylated alanine
  SITE 21: note = BSA conjugated
  source 1..21: note = paratuberculosis
AAVTTLADGGEVTWAIDGKKC

SEQ ID NO: 135 - protein, 20 aa, Mycobacterium avium
  SITE 1: note = Biotinyl
  MOD_RES 20: note = amidated aspartic acid
  source 1..20: note = paratuberculosis
KKGAAVTTLADGGEVTWAID

SEQ ID NO: 136 - protein, 20 aa, Mycobacterium avium
  SITE 1: note = Biotinyl
  MOD_RES 20: note = amidated aspartic acid
  source 1..20: note = paratuberculosis
KKGAAGTTLADGGEVTWAID

SEQ ID NO: 137 - protein, 20 aa, Mycobacterium avium
  SITE 1: note = Biotinyl
  MOD_RES 20: note = amidated aspartic acid
  source 1..20: note = paratuberculosis
KKGSTVATMADGGEVTWAID

SEQ ID NO: 138 - protein, 20 aa, Mycobacterium avium
  SITE 1: note = Biotinyl
  MOD_RES 20: note = amidated aspartic acid
  source 1..20: note = paratuberculosis
KKGQAVTRLADGGEVTWAVD

SEQ ID NO: 139 - protein, 20 aa, Mycobacterium avium
  SITE 1: note = Biotinyl
  MOD_RES 20: note = amidated leucine
  source 1..20: note = paratuberculosis
KKGFEVTTLADGTEVATSPL

SEQ ID NO: 140 - protein, 16 aa, Mycobacterium avium
  MOD_RES 15: note = Phosphoserine
  SITE 16: note = Amide BSA conjugated
  source 1..16: note = paratuberculosis
CYLSALVSIRTDPSSR

SEQ ID NO: 141 - protein, 15 aa, Mycobacterium avium
  SITE 1: note = Biotinyl
  MOD_RES 14: note = Phosphoserine
  MOD_RES 15: note = amidated arginine
  source 1..15: note = paratuberculosis
YLSALVSIRTDPSSR

SEQ ID NO: 142 - protein, 15 aa, Mycobacterium avium
  SITE 1: note = Biotinyl
  MOD_RES 15: note = amidated arginine
  source 1..15: note = paratuberculosis
YLSALVSIRTDPSSR

SEQ ID NO: 143 - protein, 15 aa, Mycobacterium avium
  SITE 1: note = Biotinyl
  MOD_RES 14: note = Phosphoserine
  MOD_RES 15: note = amidated arginine
  source 1..15: note = paratuberculosis
YLSALYSIRSDPASR

SEQ ID NO: 144 - protein, 15 aa, Mycobacterium avium
  SITE 1: note = Biotinyl
  MOD_RES 14: note = Phosphoserine
  MOD_RES 15: note = amidated arginine
  source 1..15: note = paratuberculosis
YLSALVSVRYDPSSR

SEQ ID NO: 145 - protein, 15 aa, Mycobacterium avium
  SITE 1: note = Biotinyl
  MOD_RES 14: note = Phosphoserine
  MOD_RES 15: note = amidated arginine
  source 1..15: note = paratuberculosis
YLSAQIAIRTDPASR

SEQ ID NO: 146 - protein, 42 aa, synthetic construct
  REGION 1..42: note = Synthetic polypeptide
MVINDDAQRLLSQRVDAGKADHYAVTTLADGGEVTWAIDLNA

(19) United States
(12) Patent Application Publication
(10) Pub. No.: US 2025/0152691 A1
(43) Pub. Date: May 15, 2025

(54) IMMUNOGENIC COMPOSITION FOR PARATUBERCULOSIS

(71) Applicant: HAV VACCINES LIMITED, Milton Keynes (GB)

(72) Inventor: John HERMON-TAYLOR, London (GB)

(21) Appl. No.: 18/954,032

(22) Filed: Nov. 20, 2024

Related U.S. Application Data
(62) Division of application No. 17/259,075, filed on Jan. 8, 2021, now Pat. No. 12,186,382.

(30) Foreign Application Priority Data
Jul. 11, 2018 (GB) 1811382.9

(51) Int. Cl. A61K 39/04; A61K 39/00; A61K 45/06; A61P 31/04; C07K 14/35

(52) U.S. Cl. CPC A61K 39/04; A61K 45/06; A61P 31/04; C07K 14/35; A61K 2039/5256

(57) ABSTRACT

A vaccine comprising a polypeptide comprising an amino acid sequence of at least 9 contiguous amino acids from the N-terminal region of MAP P900, or a polynucleotide encoding said polypeptide, for use in a method of treating or preventing MAP infection or a condition or symptom associated with MAP infection in a subject.

### CROSS REFERENCE TO RELATED APPLICATIONS

This application is a divisional of U.S. patent application Ser. No. 17/259,075, filed Jan. 8, 2021, now U.S. Pat. No. 12,186,382, which is a national stage application under 35 U.S.C. 371 and claims the benefit of PCT Application No. PCT/GB2019/051933 having an international filing date of 10 Jul. 2019, which designated the United States, which PCT application claimed the benefit of Great Britain Patent Application No. 1811382.9 filed 11 Jul. 2018, the disclosures of each of which are incorporated herein by reference in their entireties.

### REFERENCE TO SEQUENCE LISTING

This application contains a Sequence Listing submitted as a XML file named “ST.26 sequence listing xml. N413293US-A PXT KZX.xml”, having a size of 229,222 bytes, and created on 13 Nov. 2024. The information contained in this electronic file is hereby incorporated by reference in its entirety.

### FIELD OF THE INVENTION

The present invention relates to the treatment or prevention of infection with Mycobacterium avium subspecies paratuberculosis (MAP), and to the treatment or prevention of disorders associated with MAP infection.

### BACKGROUND TO THE INVENTION

MAP is a very slow growing intracellular mycobacterial pathogen which can cause systemic infection and chronic inflammation of the intestine (Johne's disease (JD)) in many animal species including primates. MAP was first identified in a sick dairy cow in Germany in 1894. In subsequent years MAP infection and disease appeared to be limited to Europe and North America. Since then it has spread worldwide due to international trade in subclinically infected domestic livestock and the absence of a reliable sensitive diagnostic technology able to identify MAP infection in its earliest stage. The MAP genome is GC rich (69.3%) and shares 96.4% homology in sequence and genetic organisation with closely related Mycobacterium avium and other bacteria abundant in the environment and microbiomes of animals and humans (Li et al. PNAS 2005; 102:12344-9).

MAP infected animals shed these organisms onto pastures. In pluribacillary disease the MAP pathogens in their faeces are in their common bacillary form with established red-staining Ziehl-Neelsen coats visible microscopically. Humans appear to be less susceptible to this bacillary form of MAP which is also the one commonly used in conventional partially effective whole-killed MAP vaccines. Studies have shown that exposure of children in early life to MAP-infected farm animals particularly cattle can result in a significant reduction in the subsequent incidence of Crohn's disease (Cucino C, Sonnenberg A. (2001) Am J Gastroenterol. 96:1101-5. Radon K. et al (2007) Paediatrics; 120:354-61).

Within the body of the infected animal itself MAP is present intracellularly. This form appears more aggressive to humans so that infected animal cells in milk and dairy products and meat are a source of repeated MAP infection and long term colonisation. Furthermore, MAP is a tough environmental organism and its destruction by pasteurization is incomplete. Humans are therefore at risk in dairy products from cattle, sheep and goats.

MAP survives on pastures and is taken up by protists in which intracellular environment they can survive for months or years. These organisms infect wildlife such as rabbits whose MAP-laden faeces are taken up again by grazing cattle. MAP from dead infected animals is also spread by carrion feeders. Culling of infected animals and restocking after an interval with healthy livestock merely results in the return of chronic MAP infection. Rainfall washes MAP into rivers from which MAP can spread by aerosols and directly by domestic water supplies (Richardson et al. 2019 Microorganisms 7, 136).

Once MAP becomes established in farm animals and their environment it remains and is consistently followed after an interval by the emergence and rise of Crohn's disease in the human population. The main reservoir of MAP infection is the continual replenishment of environmental sources of MAP from infected livestock. The control and subsequent reversal of the global MAP problem therefore relies on the recognition of its existence and the availability of effective vaccines which can block faecal shedding and reduce or eliminate chronic MAP infection in animals and humans. Data obtained by the U.S. department of agriculture predicts that 91.1% of U.S. dairy herds are infected with MAP (Lombard J E et al. (2013) Preventive Veterinary Medicine 108: 234-238).

The IS900 element was identified and characterised in the late 1980s (Green et al. (1989) Nucleic Acids Res; 17:9063-73). It was the first of a group of related but different DNA insertion elements found in MAP and closely related Mycobacterium avium. MAP is the only known organism with 14 to 18 identical copies of IS900. MAP is also the only pathogen known to cause chronic inflammatory diseases in so many species including primates.

A virally vectored anti-MAP vaccine has been developed by HAV Vaccines Ltd with Oxford University Jenner Institute providing the hAd5 and ChAdOx2 adenoviral priming and MVA boosting vectors containing the HAV vaccine insert (Bull et al. 2007 PLoS ONE 2(11): e1229; Bull et al. 2014 Veterinary Research 45:112). HAV Vaccines Ltd designed and made the ‘HAV’ vaccine insert, which is described in WO 2007/017635. and comprises a 95 kDa fusion construct from 4 MAP genes 1589c (AhpC), 1234 (Gsd), 2444c (p12) and 1235 (mpa) present in all MAP strains. AhpC is a secreted virulence factor in MAP shared by other pathogenic mycobacteria. Gsd is directly involved in the synthesis and transport of fucose contributing to MAP's relatively inert and highly chemical and enzymic resistance characteristics. P12 consists mostly of the extracellular carboxyterminal portion of the IS900 protein released from the mycobacterial cell and involved in pathogenicity. Mpa is a cell surface acyltransferase and may have a pore function which contributes to MAP's intrinsic resistance pattern.

Similar sequences are present within all 4 of these MAP genes in the HAV insert, to those in secondary co-pathogens in Crohn's disease, including some E. coli and other M. avium sp. Overlap of the brisk immunological responses to the HAV insert induced by vaccination are predicted to maximise the efficacy of therapeutic vaccination. The DNA of the selected HAV genes was codon optimised for mammalian cell expression and strung together to express the single HAV vaccine antigen. For patient safety this was further edited to remove any genetic sequences with homology to a mammalian sequence.

Recognition of the reality of MAP infection in humans has been delayed for years by the absence of a practical clinical MAP diagnostic (Nacy C and Buckley M. Report from the American Academy of Microbiology 2008. Mycobacterium avium paratuberculosis: Infrequent Human Pathogen or Public Health Threat?).

### SUMMARY OF THE INVENTION

The present inventor has surprisingly found that peptide fragments from the N-terminal region of the MAP P900 protein are immunogenic. Using antibodies to such peptide fragments, the inventor has shown that MAP P900 is expressed in MAP-infected human and animal subjects. The inventor has also shown that the MAP 900 protein is cleaved such that the N-terminal extracellular domain and C-terminal extracellular domain do not always co-localise in cells of infected subjects. Furthermore, the inventor has shown that it is possible to produce antibodies that are specific either for a phosphorylated N-terminal peptide or for the non-phosphorylated form of the peptide, with the antibodies being mutually exclusive. Using such phosphorylation-sensitive antibodies, the inventors have shown that the phosphorylation pattern of the N-terminus of P900 differs in infected human gut compared to infected animal gut. In the infected animals, the N-terminus is phosphorylated in the infected gut, whereas in humans, the phosphorylated N-terminus is visible only in blood cells of infected MAP subjects.

The present inventor has developed a new MAP vaccine based on the importance of P900 to MAP, the recognition of its effects on individual infected cells and populations of infected cells including the observed cleavage of the N-terminal extracellular domain and its subsequently separation and differential location to the C-terminal domain of P900. The inventor has shown that peptides within the N-terminal domain are immunogenic. These peptides and longer polypeptides comprising them, as well as polynucleotides encoding these peptides and polypeptides, may be used as vaccines. For example, the inventor has found that these peptides and polypeptides may provide additional expression stimuli in the HAV vaccine described in the art.

Accordingly, the present invention provides a vaccine comprising a polypeptide comprising an amino acid sequence of at least 9 contiguous amino acids from the region of MAP P900 shown in SEQ ID NO: 2, or a polynucleotide encoding said polypeptide, for use in a method of treating or preventing MAP infection or a condition or symptom associated with MAP infection in a subject.

In particular embodiments, the vaccine comprises a polypeptide comprising the amino acid sequence MVINDDAQRLLSQR or a polynucleotide encoding such a polypeptide. The polypeptide comprising the amino acid sequence MVINDDAQRLLSQR may be phosphorylated at the serine residue either in the vaccine composition or after administration of the vaccine to the subject. The vaccine is particularly useful for treating a subject having: Crohn's disease, Johne's disease, Ulcerative Colitis, Psoriasis, Thyroiditis, Sarcoidosis, Parkinson's disease, Multiple Sclerosis, Type 1 Diabetes, arthritis, ankylosing spondylitis, rheumatoid arthritis, irritable bowel syndrome, inflammatory bowel disease, chronic enteritis, Alzheimer's disease, multiple sclerosis, idiopathic pulmonary fibrosis, leprosy and/or chronic fatigue syndrome.

The invention further provides:
a peptide of up to 100 amino acids, which comprises the amino acid sequence: MVINDDAQRLLSQRVANDEAALLELI.
a polypeptide which comprises the amino acid sequence MVINDDAQRLLSQR and an ahpC polypeptide, a gsd polypeptide, a p12 polypeptide and/or a mpa polypeptide.
a vaccine vector comprising a comprising a polynucleotide encoding a polypeptide comprising an amino acid sequence of at least 9 contiguous amino acids from the region of MAP P900 shown in SEQ ID NO: 2.
a peptide consisting of the amino acid sequence MVINDDAQRLLSQR, or a peptide, polypeptide, polynucleotide or vaccine vector of the invention for use in a method of treating or preventing MAP infection or a condition or symptom associated with MAP infection in a subject.
a method of treating or preventing MAP infection or a condition or symptom associated with MAP infection comprising administering to a subject in need thereof an effective amount of a polypeptide comprising an amino acid sequence of at least 9 contiguous amino acids from the region of MAP P900 shown in SEQ ID NO: 2, a polynucleotide encoding said polypeptide, a peptide consisting of the amino acid sequence MVINDDAQRLLSQR, or a peptide, polypeptide, polynucleotide or vaccine vector of the invention.
a kit for use in treating or preventing MAP infection or a condition or symptom associated with MAP infection, said kit comprising (i) at least one of a polypeptide comprising an amino acid sequence of at least 9 contiguous amino acids from the region of MAP P900 shown in SEQ ID NO: 2, a polynucleotide encoding said polypeptide, a peptide consisting of the amino acid sequence MVINDDAQRLLSQR, or a peptide, polypeptide, polynucleotide or vaccine vector of the invention and (ii) at least one other therapeutic agent, for simultaneous, sequential or separate use.

### BRIEF DESCRIPTION OF THE FIGURES

FIG. 1 shows the staining of MAP ISP900 in the ileum in a 40 year old man with Crohn's disease using two specific monoclonal antibodies against Mycobacterium avium subspecies paratuberculosis (MAP). These are A1 in red (top right) and A4 in green (top left). The bottom panel shows the two together.

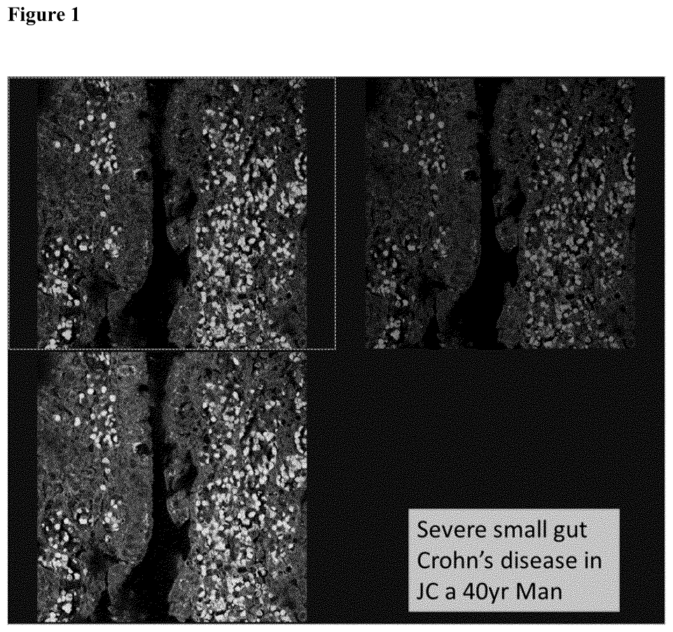

FIG. 2 shows the transverse colon of an individual with Crohn's disease stained using the A1 (red/top right) and A4 antibodies (green/top left). The bottom panel shows the two together.

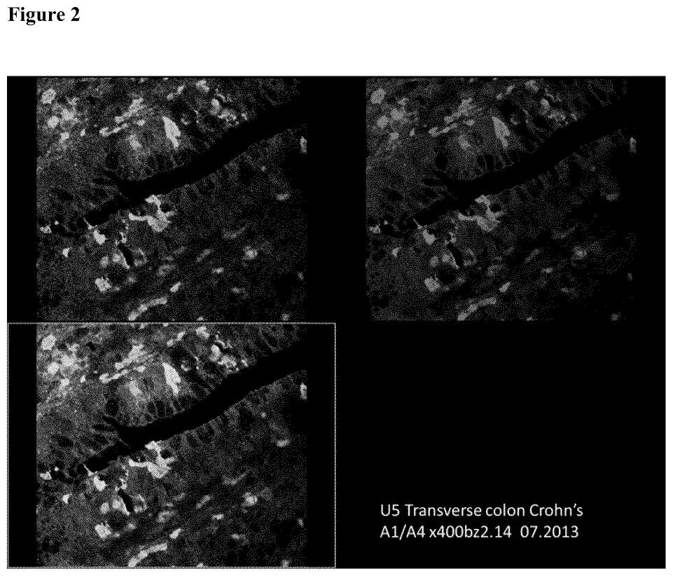

FIG. 3 shows the ileum of an individual with Crohn's disease stained using the A1 (red/top right) and A4 antibodies (green/top left). The bottom panel shows the two together.

FIG. 4 shows the rectum of a 3 month old male child with Crohn's disease stained using the A1 (red/top right) and A4 antibodies (green/top left). The bottom panel shows the two together.

FIG. 5 shows a gut arteriole in a sheep with Johne's disease stained using the A1 (red/top right) and A4 antibodies (green/top left). The bottom panel shows the two together.

FIG. 6 shows a gut arteriole in an individual with Crohn's disease stained using the A1 (red/top right) and A4 antibodies (green/top left). The bottom panel shows the two together.

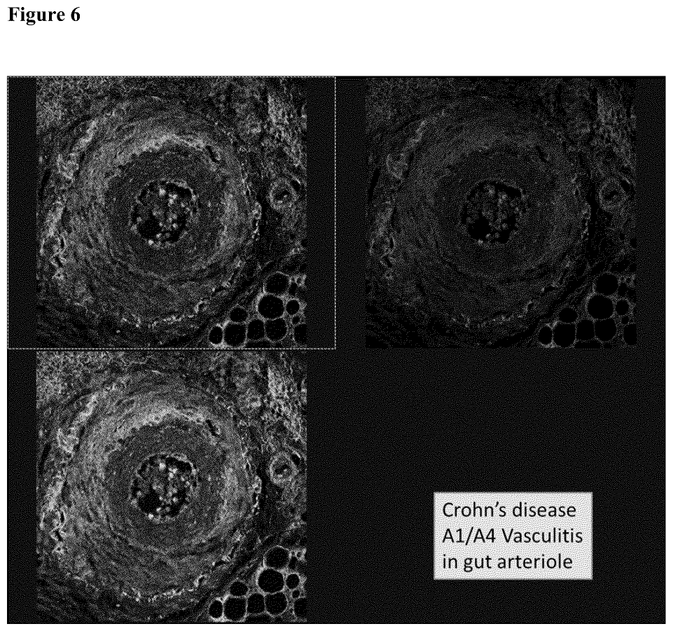

FIG. 7 shows MAP-laden white blood cells in breast milk stained using the A0X antibody.

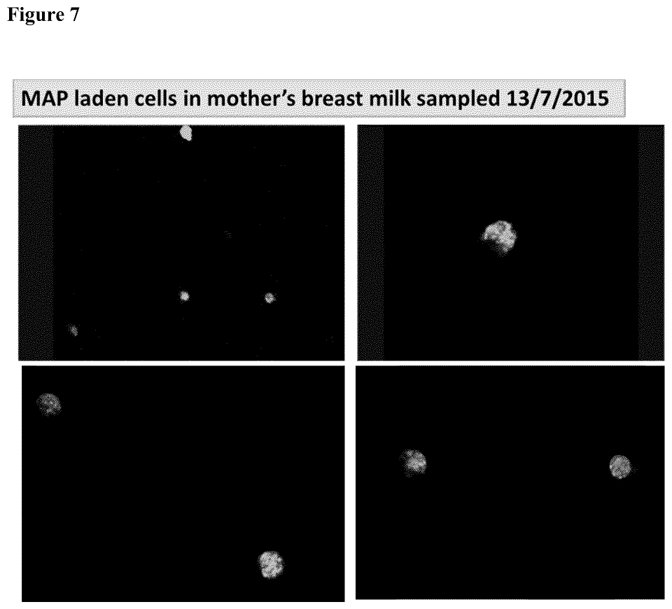

FIG. 8 shows MAP-Laden white blood cells in blood. A phase contrast image of the cells is shown (top right) and staining with the A0X antibody (top left). The bottom panel is an overlay of the two images.

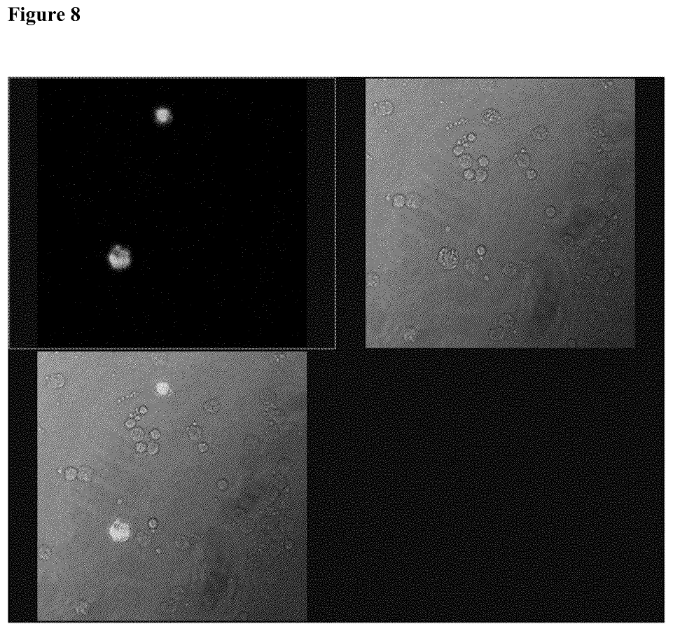

FIG. 9 shows a monocyte cell from the blood of a 25 year old man with severe Crohn's disease stained A4 in red (top right) and XA4P in green (top left). The cell is not perforated so staining is directed to the surface of the cell and almost certainly perturbs its function. The targets of the monoclonal antibodies contain the same amino acid sequence which includes 1 serine residue. This serine is not phosphorylated in the A4 target and is phosphorylated in the XA4P target. The phosphorylation event causes the targets to change their immunogenicity so that although they both crowd the cell surface in close apposition with one another they do not ad-mix. The use of A4 and XA4P monoclonal antibodies therefore allows the actions of the 2 MAP products to be used to trace the molecules and study their locations.

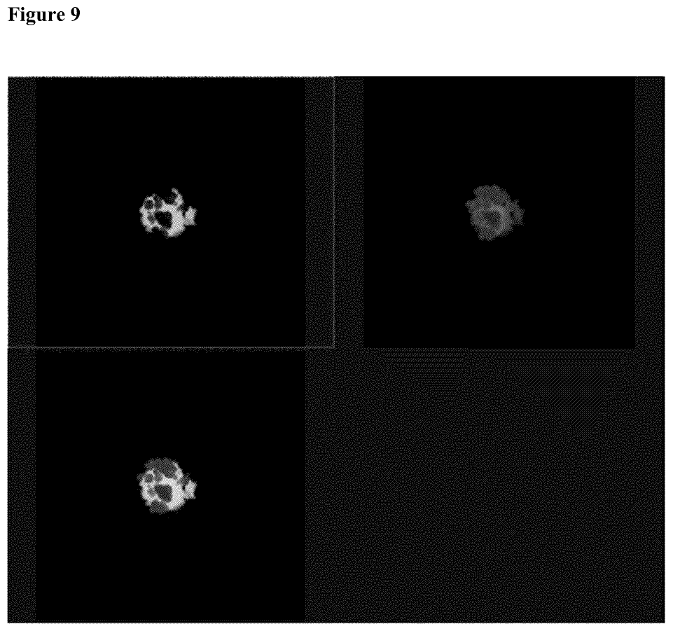

### BRIEF DESCRIPTION OF THE SEQUENCES

SEQ ID NOs: 1 and 2 are the nucleic acid and amino acid sequences respectively for the N terminal region of the MAP P900 protein.

SEQ ID NOs: 3 and 4 are the amino acid sequences of the short and long A0X peptides respectively.

SEQ ID NOs: 5 and 6 are the amino acid sequences of the short and long A0X peptides respectively, displaying the phosphorylation sites.

SEQ ID NOs: 7 and 8 are the amino acid sequences of the short and long A1 peptides respectively.

SEQ ID NOs: 9 and 10 are the amino acid sequences of the short and long AN terminal peptides respectively.

SEQ ID NOs: 11 and 12 are the amino acid sequences of the A0X short peptide linked to the A1 short peptide and the A0X short peptide linked to the A1 long peptide respectively.

SEQ ID NOs: 13 and 14 are the amino acid sequences of the A0X long peptide linked to the A1 short peptide and the A0X long peptide linked to the A1 long peptide respectively.

SEQ ID NOs: 15 and 16 are the amino acid sequences of the AN short peptide linked to the A0X short peptide and the AN short peptide linked to the A0X long peptide respectively.

SEQ ID NOs: 17 and 18 are the amino acid sequences of the AN long peptide linked to the A0X short peptide and the AN long peptide linked to the A0X long peptide respectively.

SEQ ID NOs: 19 and 20 are the amino acid sequences of the AN short peptide linked to the A0X long peptide linked to the A1 long peptide and the AN long peptide linked to the A0X long peptide respectively.

SEQ ID NOs: 21 and 22 are the nucleic acid and amino acid sequences respectively for the MAP ahpC gene

SEQ ID NOs: 23 and 24 are the nucleic acid and amino acid sequences respectively for the MAP gsd gene.

SEQ ID NOs: 25 and 26 are the nucleic acid and amino acid sequences respectively for the MAP p12 gene.

SEQ ID NOs: 27 and 28 are the nucleic acid and amino acid sequences respectively for truncated MAP p12 gene.

SEQ ID NOs: 29 and 30 are the nucleic acid and amino acid sequences respectively for the MAP mpa gene.

SEQ ID NOs: 31 and 32 are the nucleic acid and amino acid sequences respectively of a modified version of the MAP ahpC gene, in which the nucleic acid sequence has been codon optimised for human use.

SEQ ID NOs: 33 and 34 are the nucleic acid and amino acid sequences respectively of a modified version of the MAP gsd gene, in which the nucleic acid sequence has been codon optimised for human use and which is truncated at the N-terminus in order to remove the cysteine residue at position 22.

SEQ ID NOs: 35 and 36 are the nucleic acid and amino acid sequences respectively of a modified version of the MAP p12 gene, in which the nucleic acid sequence has been codon optimised for human use.

SEQ ID NOs: 37 and 38 are the nucleic acid and amino acid sequences respectively of a modified version of the truncated MAP p12 gene, in which the nucleic acid sequence has been codon optimised for human use.

SEQ ID NOs: 39 and 40 are the nucleic acid and amino acid sequences respectively of a modified version of the MAP mpa gene, in which the nucleic acid sequence has been codon optimised for human use and a number of transmembrane regions have been removed.

SEQ ID NO: 41 is the amino acid sequence of an internally truncated HAV vaccine polypeptide.

SEQ ID NO: 42 is the amino acid sequence of the HAV vaccine polypeptide.

SEQ ID NO: 43 is the amino acid sequence of the full length MAP P900 protein.

SEQ ID NO: 44 is the amino acid sequence of the A4 peptide.

SEQ ID NO: 45 is the amino acid sequence of the phosphorylated A4 peptide.

SEQ ID NO: 46 is the amino acid sequence of an alternatively phosphorylated A4 peptide.

SEQ ID NO: 47 is the amino acid sequence of the doubly phosphorylated A4 peptide.

SEQ ID NO: 48 is the amino acid sequence of the A3 peptide.

SEQ ID NO: 49 is the nucleic acid sequence of the HAV vaccine.

SEQ ID NO: 50 is the amino acid sequence of a peptide from the N-terminal region of P900.

SEQ ID NO: 51 is the amino acid sequence of a portion of the extracellular region of P900.

SEQ ID NO: 52 is the amino acid sequence of a polypeptide comprising three peptides from the extracellular region of P900.

SEQ ID NO: 53 is the amino acid sequence of a transmembrane region of P900.

SEQ ID NO: 54 is the amino acid sequence of an intramycobacterial region of P900.

SEQ ID NO: 55 is the amino acid sequence of a variant intramycobacterial region of P900.

SEQ ID NO: 56 is the amino acid sequence of a transmembrane region and an intramycobacterial region of P900.

SEQ ID NO: 57 is the amino acid sequence of a transmembrane region and a variant intramycobacterial region of P900.

SEQ ID NO: 58 is the amino acid sequence of a polypeptide comprising a portion of the extracellular region, a transmembrane region and an intramycobacterial region of P900.

SEQ ID NO: 59 is the amino acid sequence of a polypeptide comprising a portion of the extracellular region, a transmembrane region and a variant intramycobacterial region of P900.

SEQ ID NO: 60 is the amino acid sequence of a polypeptide comprising three peptides from the extracellular region, a transmembrane region and an intramycobacterial region of P900.

SEQ ID NO: 61 is the amino acid sequence of a polypeptide comprising three peptides from the extracellular region, a transmembrane region and a variant intramycobacterial region of P900.

SEQ ID NO: 62 is the amino acid sequence of the HAV vaccine polypeptide with the addition of SEQ ID NO: 59 at the N-terminus.

SEQ ID NO: 63 is the amino acid sequence of the HAVX1 vaccine polypeptide.

SEQ ID NO: 64 is the amino acid sequence of the HAV vaccine polypeptide with the addition of SEQ ID NO: 58 at the N-terminus.

SEQ ID NO: 65 is the amino acid sequence of an internally truncated HAV vaccine polypeptide with the addition of SEQ ID NO: 58 at the N-terminus.

SEQ ID NO: 61 is the amino acid sequence of the HAV vaccine polypeptide with the addition of SEQ ID NO: 59 at the N-terminus.

SEQ ID NO: 63 is the amino acid sequence of the HAVX2 vaccine polypeptide.

SEQ ID NO: 64 is the amino acid sequence of the HAV vaccine polypeptide with the addition of SEQ ID NO: 60 at the N-terminus.

SEQ ID NO: 65 is the amino acid sequence of an internally truncated HAV vaccine polypeptide with the addition of SEQ ID NO: 60 at the N-terminus.

SEQ ID NO: 70 is a consensus amino acid sequence for 2A peptides.

SEQ ID NO: 71 is the amino acid sequence of a 2A peptide.

SEQ ID NOs: 72 to 146 are amino acid sequences of peptides and polypeptides described in the Examples.

### DETAILED DESCRIPTION OF THE INVENTION

### Vaccine

The invention provides a vaccine comprising a polypeptide comprising an amino acid sequence of at least 9 contiguous amino acids from the region of MAP P900 shown in SEQ ID NO: 2, or a polynucleotide encoding said polypeptide, for use in a method of treating or preventing MAP infection or a condition or symptom associated with MAP infection in a subject.

The polypeptide in the vaccine may comprise, consist of, or consist essentially of from 9 to 71, 10 to 70, 12 to 65, 15 to 60, 18 to 55, 20 to 50, 25 to 45, or 30 to 40 contiguous amino acids of SEQ ID NO: 2.

The polypeptide may comprise two or more stretches of at least 9 contiguous amino acids from SEQ ID NO: 2. For example, the polypeptide may comprise one, two, three or more stretches of 9 or more contiguous amino acids from the amino acid sequence of SEQ ID NO: 2. Each contiguous stretch of amino acids may be a stretch of from 9 to 30 amino acids, such as from 12 to 25, 13 to 24, 14 to 22 or 15 to 20 amino acids in length. The contiguous amino acids from SEQ ID NO: 2 may be joined directly to each other or may comprise a linker between the stretches of contiguous amino acids. Preferably, a peptide linker (e.g. another amino acid sequence) is used to join the contiguous stretches of amino acids.

The vaccine may comprise a polypeptide comprising, consisting of, or consisting essentially of the amino acid sequence of SEQ ID NO: 3 (MVINDDAQRLLSQR) and/or any one or more of the peptides or polypeptides as described below alone or in combination. Specifically the vaccine may comprise a polypeptide comprising, consisting of or consisting essentially of at least one of the following amino acid sequences: MVINDDAQRLLSQR (SEQ ID NO: 3), MVINDDAQRLLSQRVANDEAALLELI (SEQ ID NO: 4), MVINDDAQRLL[pS]QR (SEQ ID NO: 5), VTTLADGGEVTWAID (SEQ ID NO: 7), VTTLADGGEVTWAIDLNA (SEQ ID NO: 8), EVVVAQPVWAGVDAGKADHY (SEQ ID NO: 9), MTVTEVVVAQPVWAGVDAGKADHY (SEQ ID NO: 10) and VDAGKADHY (SEQ ID NO: 50). Where two or more of these amino acid sequences are present in the polypeptide, they may be joined directly to one another, or the polypeptide may comprise one or more additional linking amino acids, such as from 1 to 20, 2 to 15, 3 to 10 or 4 to 8 amino acids. For example, A or AA may be used to join the amino terminus of

(SEQ ID NO: 7)
VTTLADGGEVTWAID,
(SEQ ID NO: 8)
VTTLADGGEVTWAIDLNA

to another amino acid sequence. The amino acids may be joined in any order. In some embodiments they are joined in the order that they occur in P900.

In some embodiments, the polypeptide in the vaccine, or encoded by the vaccine, comprises the amino acid sequence of any two or all of SEQ ID NO: 10, SEQ ID NO: 4 and SEQ ID NO: 7 in any order. These sequences may be immediately adjacent to one another, or may include one or more additional amino acids joining them. In some embodiments SEQ ID NO: 10, SEQ ID NO: 4 and SEQ ID NO: 7 may be joined in this order. In these cases the polypeptide may comprise the amino acid sequence of the whole of the normal extra-mycobacterial region:

(SEQ ID NO: 51)
MTVTEVVVAQPVWAGVDAGKADHYCMVINDDAQRLLSQRVANDEAALLEL
IAAVTTLADGGEVTWAID.

In some embodiments, the polypeptide in the vaccine, or encoded by the vaccine, comprises the amino acid sequence of SEQ ID NO: 3, SEQ ID NO:50 and SEQ ID NO: 7 in any order. For example, SEQ ID NO: 3, SEQ ID NO:50 and SEQ ID NO: 7 may be joined in this order. These sequences may be immediately adjacent to one another, or may include one or more additional amino acids joining them. For example, the polypeptide may comprise the amino acid sequence:

(SEQ ID NO: 52)
MVINDDAQRLLSQRVDAGKADHYAVTTLADGGEVTWAID.

In some embodiments, the vaccine may additionally comprise an amino acid sequence that forms all or part of a transmembrane stretch of P900. For example, the vaccine may comprise a polypeptide comprising the amino acid sequence

(SEQ ID NO: 53)
LNAGGAALLIALLIAAGQRLLY

in addition to one or more of the amino acid sequences mentioned above. The polypeptide may further comprise one or more amino acids that are adjacent to the transmembrane stretch on the cytoplasmic side of the membrane. For example, the vaccine may comprise the amino acid sequence IPGRTVHHAAGSYRGE (SEQ ID NO: 54), the variant thereof, IPGATVHHAAGSYRGE (SEQ ID NO: 55), or any N-terminal fragment of either thereof. In the polypeptide, the amino acid sequence SEQ ID NO: 54 or SEQ ID NO: 55, or the N-terminal fragment thereof, is preferably adjacent to the C-terminal end of the amino acid sequence of SEQ ID NO: 53. For example, the polypeptide may comprise the amino acid sequence

(SEQ ID NO: 56)
LNAGGAALLIALLIAAGQRLLYIPGRTVHHAAGSYRGE
(SEQ ID NO: 57)
LNAGGAALLIALLIAAGQRLLYIPGATVHHAAGSYRGE.

The N-terminal fragment of SEQ ID NO: 54 or SEQ ID NO: 55, may be from 1 to 15 amino acids in length, such as from 2 to 14, 3 to 10, 4 to 9, 5 to 8, 6 or 7 amino acids in length.

The vaccine may, for example, include or encode a polypeptide comprising the amino acid sequence:

(SEQ ID NO: 58)
MTVTEVVVAQPVWAGVDAGKADHYCMVINDDAQRLLSQRVANDEAALLEL
IAAVTTLADGGEVTWAIDLNAGGAALLIALLIAAGQRLLYIPGRTVHHAA
GSYRGE;
(SEQ ID NO: 59)
MTVTEVVVAQPVWAGVDAGKADHYCMVINDDAQRLLSQRVANDEAALLEL
IAAVTTLADGGEVTWAIDLNAGGAALLIALLIAAGQRLLYIPGATVHHAA
GSYRGE
(SEQ ID NO: 60)
MVINDDAQRLLSQRVDAGKADHYAVTTLADGGEVTWAIDLNAGGAALLIA
LLIAAGQRLLYIPGRTVHHAAGSYRGE;
or
(SEQ ID NO: 61)
MVINDDAQRLLSQRVDAGKADHYAVTTLADGGEVTWAIDLNAGGAALLIA
LLIAAGQRLLYIPGATVHHAAGSYRGE.

SEQ ID NO: 58 and SEQ ID NO: 59 represent the normal extra-mycobacterial region, the transmembrane region (SEQ ID NO: 53) and the first intra-mycobacterial portion of the P900 sequence (SEQ ID NO: 54 or SEQ ID NO: 55) stopping short of the active site mechanism of the putative transposase. It has no known toxicity and is abundantly expressed in vivo during MAP infection. SEQ ID NO: 60 and SEQ ID NO: 61 disrupt the normal sequence of P900, place emphasis on the N-terminal epitope (SEQ ID NO: 3) and expose a further epitope (SEQ ID NO: 7/SEQ ID NO: 8) just before it dips into the transmembrane sequence (SEQ ID NO: 53). The present inventor has shown for the first time that the extracellular amino terminal end of P900 comprises at least three immunogenic regions. These immunogenic regions are SEQ ID NOs: 3 and 4 (referred to herein as A0X), which can be phosphorylated to form SEQ ID NOs: 5 and 6 (herein referred to as A0XP); the SEQ ID NOs: 7 and 8 (herein referred to as A1); and SEQ ID NOs: 9 and 10 (referred to herein as AN).

In a preferred embodiment, the vaccine comprises the A0X peptide, or the A0X peptide is coded in the vaccine sequence and expressed in the native amino acid form. After vaccination and expression the serine may be phosphorylated as shown. Both phosphorylated and non-phosphorylated forms of the peptide can be present within the same MAP infected cells and on the same cell surfaces. Similarly, vaccinated subjects may present both the phosphorylated and un-phosphorylated form to the immune system. Staining of such cells with fluorescent monoclonal antibodies specific for either the native peptide or its phosphorylated derivative shows that the antibodies are mutually exclusive and reveal separate labelled peptide clusters packing the cytoplasm and surface of MAP-infected cells. Despite being widely exposed to antibody and cell mediated immunity these mycobacterial peptides are not recognised by the unvaccinated MAP infected host. Vaccination releases the block on immune recognition of the amino terminal peptides as it does after vaccination at the carboxyterminal portion of P900.

There are important species differences in A0X serine phosphorylation. Monoclonal antibodies to A0XP were made by immunising mice to synthetic MVINDDAQRLLpSQR and clonal selection. These antibodies demonstrated that phosphorylated A0XP could not be seen in sections of normal or inflamed human intestinal tissue except faintly in immune cells within intestinal blood vessels. A0XP is however, strongly positive against circulating human WBCs. On the other hand in cattle, sheep, goats and deer A0XP was strongly positive in intestinal cells as well in circulating WBCs. These new observations may be taken into account when considering the use of additional immunisation against MAP infection in animals using synthetic polymers of A0XP and appropriate adjuvants.

In a preferred embodiment, the vaccine comprises the A0X peptide, or the A0X peptide is coded in the vaccine sequence and expressed in the native amino acid form and the A0X peptide is not adjacent to a cysteine residue at its N-terminal end. For example, the A0X peptide is at the N-terminus of the peptide or polypeptide in the vaccine. The cysteine residue present next to the A0X peptide amino acid sequence in P900 is thought to tether P900 to the membrane. Expressing the A0X peptide without this N-terminal cysteine residue improves the visibility of the peptide to the immune system.

The vaccine may comprise A1 peptide, optionally in addition to the A0X peptide. The A1 peptide is closely attached to the external surface of MAP and its carboxy terminus dives into the outer layer of its first transmembrane portion (highlighted above). It is a robust antigen and a useful marker for MAP and closely related M. avium. For greatest immunogenicity, the two alanine residues present in the P900 amino acid sequence adjacent to the amino terminus of A1 are not included in A1 such that the peptide immunogen begins with VTT. However, the A1 peptide may additionally include two N-terminal alanine residues. The present inventor discovered that A1 is an immunogen in mice and rabbits immunised with truncated recombinant P900 followed by screening against a cascade of synthetic 15mer peptide antigens.

The vaccine may comprise the AN peptide, optionally in addition to the A0X and/or A1 peptide. Unlike the other amino terminal peptides of P900 spontaneous antibody recognition of the AN peptide sequence although low is generally seen in MAP infected hosts. If using the synthetic AN peptide as a vaccine immunogen omit the initial MTVT to improve immunogenicity. If using the peptide as an immunogen the c-terminal Cys can be removed to prevent cross linking or used with a maleimido linkage to a carrier molecule such as Keyhole Limpet Haemocyanin (KLH) or bovine serum albumin.

Particular examples of polypeptides that may be used include polypeptides comprising one of the following amino acid sequences:

(i)
(SEQ ID NO: 11)
MVINDDAQRLLSQRX₂VTTLADGGEVTWAID;
(ii)
(SEQ ID NO: 12)
MVINDDAQRLLSQRX₂VTTLADGGEVTWAIDLNA;
(iii)
(SEQ ID NO: 13)
MVINDDAQRLLSQRVANDEAALLELIX₂VTTLADGGEVTWAID;
(iv)
(SEQ ID NO: 14)
MVINDDAQRLLSQRVANDEAALLELIX₂VTTLADGGEVTWAIDLNA;
(v)
(SEQ ID NO: 15)
EVVVAQPVWAGVDAGKADHYX₁MVINTDDAQRLLSQR;
(vi)
(SEQ ID NO: 16)
EVVVAQPVWAGVDAGKADHYX₁MVINTDDAQRLLSQRVANDEAALLELI;
(vii)
(SEQ ID NO: 17)
MTVTEVVVAQPVWAGVDAGKADHYX₁MVINDDAQRLLSQR;
or
(viii)
(SEQ ID NO: 18)
MTVTEVVVAQPVWAGVDAGKADHYX₁MVINDDAQRLLSQRVANDEAALLE
LI,
(ix)
(SEQ ID NO: 19)
EVVVAQPVWAGVDAGKADHYX₁MVINDDAQRLLSQRVANDEAALLELIX₂
VTTLADGGEVTWAIDLNA;
and
(x)
(SEQ ID NO: 20)
MTVTEVVVAQPVWAGVDAGKADHYX₁MVINDDAQRLLSQRVANDEAALLE
LIX₂VTTLADGGEVTWAIDLNA.

wherein X₁ is a peptide linker or C and X₂ is a peptide linker, A or AA. The peptide linker may be any of the peptide linkers as described below.

The polypeptide may be a variant of SEQ ID NO: 2 that comprises one or more of SEQ ID NOs: 3 to 10 and has at least one amino acid substitution, deletion or addition in SEQ ID NO: 2 outside the region of the amino acid sequence of SEQ ID NOs: 3, 4, 5, 6, 7, 8, 9 or 10.

The polypeptide in the vaccine may further comprise the amino acid sequence of at least one additional MAP polypeptide, or a fragment thereof. The vaccine may further comprise the amino acid sequence of at least one additional MAP polypeptide, or a fragment thereof or at least one further polynucleotide encoding an additional MAP polypeptide, or a fragment thereof.

The polypeptide described above, may, in one embodiment, be joined to the additional polypeptide by a “self-cleaving” peptide. A “self-cleaving” peptide is a peptide that mediates cleavage of the polypeptide it is contained in during translation. One example of a self cleaving peptide is a 2A polypeptide. 2A peptides are 18-22 amino acid-long viral peptides that mediate cleavage of polypeptides during translation in eukaryotic cells (Liu et al. 2017 Scientific Reports 7:2193). The 2A peptide may be derived from any virus that includes a 2A peptide in its viral genome. The 2A peptide typically comprises the conserved sequence GDVEXNPGP (SEQ ID NO: 70). One example of a 2A peptide has the sequence APVKQTLNFDLLKLAGDVESNPGP (SEQ ID NO: 71).

The least one additional polypeptide may be, for example, an ahpC polypeptide, a gsd polypeptide, a p12 polypeptide and/or a mpa polypeptide. In particular, expression of a nucleic acid sequence encoding the extracellular amino terminal P900 peptides of P900 will facilitate the disabling of the P900 system when used in combination with a p12 polypeptide, for example when introduced into the HAV vaccine described in WO2007/017635, which is incorporated herein by reference in its entirety.

ahpC is a secreted component shared by many pathogenic mycobacteria. It is involved in the ability of MAP to survive within macrophages and is upregulated on entry into a state of microbial dormancy. The nucleic acid and amino acid sequences of the MAP ahpC gene and protein are given in SEQ ID NOs: 21 and 22 respectively. For use in the present invention, this sequence, or a variant thereof as discussed below may be used. For example, the MAP ahpC gene sequence may be codon optimised as discussed further below to make it more suitable for mammalian, in particular human, use. A suitable modified ahpC sequence and encoded protein are given in SEQ ID NOs: 31 and 32 respectively.

gsd is a glycosyl transferase encoded by the GS pathogenicity element with a predicted signal sequence and lipid acylation site. Microarray analysis shows that it is up-regulated in the intracellular environment. It is expressed on the microbial cell surface and is predicted to transfer GDP-fucose to sub-terminal rhamnose to cap surface glycopeptidolipid on MAP with derivatised fucose giving the pathogen in its ZN-negative state an inert, hydrophobic, and highly resistant cell surface. The nucleic acid and amino acid sequences of the MAP gsd gene and protein are given in SEQ ID NOs: 23 and 24 respectively. For use in the present invention, this sequence, or a variant thereof as discussed below, may be used. For example, the MAP gsd gene sequence may be codon optimised as discussed further below to make it more suitable for mammalian, in particular human, use. Other modifications may be made, for example potential acylation sites may be removed. One suitable modified gsd sequence and encoded protein are given in SEQ ID NOs: 33 and 34 respectively.

p12 is the carboxyterminal 17 kDa fragment of p43 encoded by IS900 which is also up-regulated intracellularly. It is strongly predicted on the cell surface and both in MAP and in p43.rec.E. coli it is the substrate for specific proteolytic cleavage and exodomain release. The nucleic acid and amino acid sequences of the MAP p12 gene and protein are given in SEQ ID NOs: 25 and 26 respectively. For use in the present invention, this sequence, or a variant thereof as discussed below may be used. For example, the MAP p12 gene sequence may be codon optimised as discussed further below to make it more suitable for mammalian, in particular human, use. One suitable modified p12 sequence and encoded protein are given in SEQ ID NOs: 35 and 36 respectively.

mpa is also expressed on the surface of MAP and is believed to be unique to the pathogen. It is both an acetylase and a predicted pore molecule with 10 transmembrane regions and a large extracellular peptide loop. The nucleic acid and amino acid sequences of the MAP mpa gene and protein are given in SEQ ID NOs: 29 and 30 respectively. For use in the present invention, this sequence, or a variant thereof as discussed below may be used. For example, the MAP mpa gene sequence may be codon optimised as discussed further below to make it more suitable for mammalian, in particular human, use. Other modifications may be made, for example transmembrane regions may be removed to reduce the hydrophobicity of the protein. One suitable modified mpa sequence and encoded protein are given in SEQ ID NOs: 39 and 40 respectively.

A suitable ahpC polypeptide may have the amino acid sequence of SEQ ID NO: 22 or SEQ ID NO: 32. A suitable gsd polypeptide may have the amino acid sequence of SEQ ID NO: 24 or SEQ ID NO: 34. A suitable p12 polypeptide may have the amino acid sequence of SEQ ID NO: 26 or SEQ ID NO: 36. A suitable mpa polypeptide may have the amino acid sequence of SEQ ID NO: 30 or SEQ ID NO: 40. A suitable ahpC, gsd, p12 or mpa sequence may alternatively be a variant of one of these specific sequences. For example, a variant may be a substitution, deletion or addition variant of any of the above amino acid sequences, or may be a fragment of any thereof as described herein.

In particular embodiments, the ahpC polypeptide may comprise the sequence of SEQ ID NO: 22, a variant thereof having more than 70% amino acid sequence identity to SEQ ID NO: 22 across the full length of SEQ ID NO: 22, or a fragment of at least 8 amino acids of SEQ ID NO: 22 which comprises an epitope. Preferably the ahpC polypeptide has the amino acid sequence given in SEQ ID NO: 32. The gsd polypeptide may comprise the sequence of SEQ ID NO: 24, a variant thereof having more than 70% amino acid sequence identity to SEQ ID NO: 24 across the full length of SEQ ID NO: 24, or a fragment of at least 8 amino acids of SEQ ID NO: 24 which comprises an epitope. Preferably the gsd polypeptide has the amino acid sequence given in SEQ ID NO: 34. The pl2 polypeptide may comprise the sequence of SEQ ID NO: 26, a variant thereof having more than 70% amino acid sequence identity to SEQ ID NO: 26 across the full length of SEQ ID NO: 26, or a fragment of at least 8 amino acids of SEQ ID NO: 26 which comprises an epitope. The fragment of SEQ ID NO: 10 preferably comprises SEQ ID NOs: 44, 45, 46 or 47 and/or SEQ ID NO: 48. Preferably the pl2 polypeptide has the amino acid sequence given in SEQ ID NO: 36 or SEQ ID NO: 37 or SEQ ID NOs: 44, 45, 46 or 47 and/or SEQ ID NO: 48. The mpa polypeptide may comprise the sequence of SEQ ID NO: 30, a variant thereof having more than 70% amino acid sequence identity to SEQ ID NO: 30 across the full length of SEQ ID NO: 30, or a fragment of at least 8 amino acids of SEQ ID NO: 30 which comprises an epitope. Preferably the mpa polypeptide has the amino acid sequence given in SEQ ID NO: 40.

The introduction of an extracellular amino terminal peptides of the P900 protein into a MAP vaccine comprising a p12 polypeptide acts to complete the disabling of both ends of the P900 protein system. Therefore, in a preferred embodiment, the vaccine comprises a polypeptide comprising at least 9 contiguous amino acids of SEQ ID NO: 2 and a polypeptide comprising at least 9 contiguous amino acids of SEQ ID NO: 10, or a polypeptide comprising at least 9 contiguous amino acids of SEQ ID NO: 2 and at least 9 contiguous amino acids of SEQ ID NO: 10, or a polynucleotide, or polynucleotides, encoding said polypeptide(s). For example, the vaccine may comprise a polypeptide comprising, consisting of, or consisting essentially of any one of SEQ ID NOs: 3 to 10 and a polypeptide comprising, consisting of, or consisting essentially of any one of SEQ ID NOs: 44 to 48, or a polypeptide comprising at least one of SEQ ID NOs: 3 to 10 and at least one of SEQ ID NOs: 44 to 48, or a polynucleotide, or polynucleotides, encoding said polypeptide(s). In a preferred embodiment the vaccine comprises a polypeptide comprising, consisting of, or consisting essentially SEQ ID NO: 3 or 4 and a polypeptide comprising, consisting of, or consisting essentially of any one of SEQ ID NOs: 44 to 47, or a polypeptide comprising SEQ ID NO: 3 or 4 and one of SEQ ID NOs: 44 to 48, or a polynucleotide, or polynucleotides, encoding said polypeptide(s).

In particular embodiments, the vaccine may comprise a polypeptide comprising, consisting of or consisting essentially of the amino acid sequence: MQIFVKLPLLTIGDQFPAYELTALIAGDLSKVDAKQPGDYFTTVTSEDHAGKWRV VFFWPKDFTGPEIATFGKLNDEFEDRDAQVLGVSIDSEFVHFNWRAQHEDLKNLP FPMLSDIKRELSLATGVLNADGVADRATFIVDPNNEIQFVSVTAGSVGRNVEEVLR VLDALQSDELCACNWRKGDPTLNATELLKASALGSIVGQTYREVEVVLVDGGST DRTLDIANSFRPELGSRLVVHSGPDDGPYDAMNRGVGVATGEWVLFLGADDTLY EPTTLAQVAAFLGDHAASHLVYGDVVMRSTKSRHAGPFDLDRLLFETNLCHQSIF YRRELFDGIGPYNLRYRVWADWDFNIRCFSNPALITRYMDVVISEYNDMTGFSMR QGTDKEFRKRLPMYFWVAGWETCRRMLAFLKDKENRRLALRTRLIRVKAVSKER SAEPRIRRHRHAEIILSMPGFGVILGAEFLAATGGDMAAFASADRLAGVAGLAPVP RDSGRISGNLKRPRRYDRRLLRACVSIRTDPSSRTYYDRKRTEGKRHTQAVLALAR RRLNVLWAMLRDHAVYHPATTTAAARLKLRRGERPMSLGQVFDPRANALHSFPL TGRMPWAPFIVSSWLRNPHPAQYFTARCLRILPGLWIGAQGGSAAKLLMSGAPIE YVLKDSAVWMFKFDIGGTPRDIPVAGIWNGSLWTPAWGGIHAIASNAYQFRNVIP ARWSVSSAVLPNYRLVAALPMAYHNQRMRFRTDLSYGVYGFAEINPIALVEKPAL SWKSRLRRKNSSIALANMEDGGSVGRSNDIPGRRARFIGEKAEDPPAPSPRPALRI PNPLLGLD (SEQ ID NO: 41). The vaccine may comprise a polypeptide comprising, consisting of or consisting essentially of the amino acid sequence shown above (SEQ ID NO: 41), wherein a peptide comprising an amino acid sequence of at least 9 contiguous amino acids from the region of MAP P900 shown in SEQ ID NO: 2, preferably a peptide comprising, consisting of or consisting essentially of the amino acid sequence MVINDDAQRLLSQR, is added at the N-terminus or inserted between: positions 7 and 8, positions 199 and 200, positions 442 and 443, positions 577 and 578, and/or positions 820 and 821; or MQIFVKLPLLTIGDQFPAYELTALIAGDLSKVDAKQPGDYFTTVTSEDHAGKWRV VFFWPKDFTGPEIATFGKLNDEFEDRDAQVLGVSIDSEFVHFNWRAQHEDLKNLP FPMLSDIKRELSLATGVLNADGVADRATFIVDPNNEIQFVSVTAGSVGRNVEEVLR VLDALQSDELCACNWRKGDPTLNATELLKASALGSIVGQTYREVEVVLVDGGST DRTLDIANSFRPELGSRLVVHSGPDDGPYDAMNRGVGVATGEWVLFLGADDTLY EPTTLAQVAAFLGDHAASHLVYGDVVMRSTKSRHAGPFDLDRLLFETNLCHQSIF YRRELFDGIGPYNLRYRVWADWDFNIRCFSNPALITRYMDVVISEYNDMTGFSMR QGTDKEFRKRLPMYFWVAGWETCRRMLAFLKDKENRRLALRTRLIRVKAVSKER SAEPRIRRHRHAEIILSMPGFGVILGAEFLAATGGDMAAFASADRLAGVAGLAPVP RDSGRISGNLKRPRRYDRRLLRACYLSALVSIRTDPSSRTYYDRKRTEGKRHTQAV LALARRRLNVLWAMLRDHAVYHPATTTAAARLKLRRGERPMSLGQVFDPRANA LHSFPLTGRMPWAPFIVSSWLRNPHPAQYFTARCLRILPGLWIGAQGGSAAKLLMS GAPIEYVLKDSAVWMFKFDIGGTPRDIPVAGIWNGSLWTPAWGGIHAIASNAYQF RNVIPARWSVSSAVLPNYRLVAALPMAYHNQRMRFRTDLSYGVYGFAEINPIALV EKPALSWKSRLRRKNSSIALANMEDGGSVGRSNDIPGRRARFIGEKAEDPPAPSPR PALRIPNPLLGLD (SEQ ID NO: 42) wherein a peptide comprising an amino acid sequence of at least 9 contiguous amino acids from the region of MAP P900 shown in SEQ ID NO: 2, preferably a peptide comprising, consisting of or consisting essentially of amino acid sequence MVINDDAQRLLSQR, is added at the N-terminus or inserted between: positions 7 and 8, positions 199 and 200, positions 442 and 443, positions 582 and 583,and/or positions 825 and 826.

In particular embodiments, the vaccine may comprise a polypeptide comprising, consisting of or consisting essentially of the amino acid sequence:

(SEQ ID NO: 62)
MTVTEVVVAQPVWAGVDAGKADHYCMVINDDAQRLLSQRVANDEAALL
ELIAAVTTLADGGEVTWAIDLNAGGAALLIALLIAAGQRLLYIPGATV
HHAAGSYRGEAPVKQTLNFDLLKLAGDVESNPGPMQIFVKLPLLTIGD
QFPAYELTALIAGDLSKVDAKQPGDYFTTVTSEDHAGKWRVVFFWPKD
FTGPEIATFGKLNDEFEDRDAQVLGVSIDSEFVHFNWRAQHEDLKNLP
FPMLSDIKRELSLATGVLNADGVADRATFIVDPNNEIQFVSVTAGSVG
RNVEEVLRVLDALQSDELCACNWRKGDPTLNATELLKASALGSIVGQT
YREVEVVLVDGGSTDRTLDIANSFRPELGSRLVVHSGPDDGPYDAMNR
GVGVATGEWVLFLGADDTLYEPTTLAQVAAFLGDHAASHLVYGDVVMR
STKSRHAGPFDLDRLLFETNLCHQSIFYRRELFDGIGPYNLRYRVWAD
WDFNIRCFSNPALITRYMDVVISEYNDMTGFSMRQGTDKEFRKRLPMY
FWVAGWETCRRMLAFLKDKENRRLALRTRLIRVKAVSKERSAEPRIRR
HRHAEIILSMPGFGVILGAEFLAATGGDMAAFASADRLAGVAGLAPVP
RDSGRISGNLKRPRRYDRRLLRACYLSALVSIRTDPSSRTYYDRKRTE
GKRHTQAVLALARRRLNVLWAMLRDHAVYHPATTTAAARLKLRRGERP
MSLGQVFDPRANALHSFPLTGRMPWAPFIVSSWLRNPHPAQYFTARCL
RILPGLWIGAQGGSAAKLLMSGAPIEYVLKDSAVWMFKFDIGGTPRDI
PVAGIWNGSLWTPAWGGIHAIASNAYQFRNVIPARWSVSSAVLPNYRL
VAALPMAYHNQRMRFRTDLSYGVYGFAEINPIALVEKPALSWKSRLRR
KNSSIALANMEDGGSVGRSNDIPGRRARFIGEKAEDPPAPSPRPALRI
PNPLLGLD;
(SEQ ID NO: 63)
MTVTEVVVAQPVWAGVDAGKADHYCMVINDDAQRLLSQRVANDEAALL
ELIAAVTTLADGGEVTWAIDLNAGGAALLIALLIAAGQRLLYIPGATV
HHAAGSYRGEAPVKQTLNFDLLKLAGDVESNPGPMQIFVKLPLLTIGD
QFPAYELTALIAGDLSKVDAKQPGDYFTTVTSEDHAGKWRVVFFWPKD
FTGPEIATFGKLNDEFEDRDAQVLGVSIDSEFVHFNWRAQHEDLKNLP
FPMLSDIKRELSLATGVLNADGVADRATFIVDPNNEIQFVSVTAGSVG
RNVEEVLRVLDALQSDELCACNWRKGDPTLNATELLKASALGSIVGQT
YREVEVVLVDGGSTDRTLDIANSFRPELGSRLVVHSGPDDGPYDAMNR
GVGVATGEWVLFLGADDTLYEPTTLAQVAAFLGDHAASHLVYGDVVMR
STKSRHAGPFDLDRLLFETNLCHQSIFYRRELFDGIGPYNLRYRVWAD
WDFNIRCFSNPALITRYMDVVISEYNDMTGFSMRQGTDKEFRKRLPMY
FWVAGWETCRRMLAFLKDKENRRLALRTRLIRVKAVSKERSAEPRIRR
HRHAEIILSMPGFGVILGAEFLAATGGDMAAFASADRLAGVAGLAPVP
RDSGRISGNLKRPRRYDRRLLRACVSIRTDPSSRTYYDRKRTEGKRHT
QAVLALARRRLNVLWAMLRDHAVYHPATTTAAARLKLRRGERPMSLGQ
VFDPRANALHSFPLTGRMPWAPFIVSSWLRNPHPAQYFTARCLRILPG
LWIGAQGGSAAKLLMSGAPIEYVLKDSAVWMFKFDIGGTPRDIPVAGI
WNGSLWTPAWGGIHAIASNAYQFRNVIPARWSVSSAVLPNYRLVAALP
MAYHNQRMRFRTDLSYGVYGFAEINPIALVEKPALSWKSRLRRKNSSI
ALANMEDGGSVGRSNDIPGRRARFIGEKAEDPPAPSPRPALRIPNPLL
GLD;
(SEQ ID NO: 64)
MTVTEVVVAQPVWAGVDAGKADHYCMVINDDAQRLLSQRVANDEAALL
ELIAAVTTLADGGEVTWAIDLNAGGAALLIALLIAAGQRLLYIPGRTV
HHAAGSYRGEAPVKQTLNFDLLKLAGDVESNPGPMQIFVKLPLLTIGD
QFPAYELTALIAGDLSKVDAKQPGDYFTTVTSEDHAGKWRVVFFWPKD
FTGPEIATFGKLNDEFEDRDAQVLGVSIDSEFVHFNWRAQHEDLKNLP
FPMLSDIKRELSLATGVLNADGVADRATFIVDPNNEIQFVSVTAGSVG
RNVEEVLRVLDALQSDELCACNWRKGDPTLNATELLKASALGSIVGQT
YREVEVVLVDGGSTDRTLDIANSFRPELGSRLVVHSGPDDGPYDAMNR
GVGVATGEWVLFLGADDTLYEPTTLAQVAAFLGDHAASHLVYGDVVMR
STKSRHAGPFDLDRLLFETNLCHQSIFYRRELFDGIGPYNLRYRVWAD
WDFNIRCFSNPALITRYMDVVISEYNDMTGFSMRQGTDKEFRKRLPMY
FWVAGWETCRRMLAFLKDKENRRLALRTRLIRVKAVSKERSAEPRIRR
HRHAEIILSMPGFGVILGAEFLAATGGDMAAFASADRLAGVAGLAPVP
RDSGRISGNLKRPRRYDRRLLRACYLSALVSIRTDPSSRTYYDRKRTE
GKRHTQAVLALARRRLNVLWAMLRDHAVYHPATTTAAARLKLRRGERP
MSLGQVFDPRANALHSFPLTGRMPWAPFIVSSWLRNPHPAQYFTARCL
RILPGLWIGAQGGSAAKLLMSGAPIEYVLKDSAVWMFKFDIGGTPRDI
PVAGIWNGSLWTPAWGGIHAIASNAYQFRNVIPARWSVSSAVLPNYRL
VAALPMAYHNQRMRFRTDLSYGVYGFAEINPIALVEKPALSWKSRLRR
KNSSIALANMEDGGSVGRSNDIPGRRARFIGEKAEDPPAPSPRPALRI
PNPLLGLD;
(SEQ ID NO: 65)
MTVTEVVVAQPVWAGVDAGKADHYCMVINDDAQRLLSQRVANDEAALL
ELIAAVTTLADGGEVTWAIDLNAGGAALLIALLIAAGQRLLYIPGRTV
HHAAGSYRGEAPVKQTLNFDLLKLAGDVESNPGPMQIFVKLPLLTIGD
QFPAYELTALIAGDLSKVDAKQPGDYFTTVTSEDHAGKWRVVFFWPKD
FTGPEIATFGKLNDEFEDRDAQVLGVSIDSEFVHFNWRAQHEDLKNLP
FPMLSDIKRELSLATGVLNADGVADRATFIVDPNNEIQFVSVTAGSVG
NVEEVLRVLDALQSDELCACNWRKGDPTLNATELLKASALGSIVGQT
YREVEVVLVDGGSTDRTLDIANSFRPELGSRLVVHSGPDDGPYDAMNR
GVGVATGEWVLFLGADDTLYEPTTLAQVAAFLGDHAASHLVYGDVVMR
STKSRHAGPFDLDRLLFETNLCHQSIFYRRELFDGIGPYNLRYRVWAD
WDFNIRCFSNPALITRYMDVVISEYNDMTGFSMRQGTDKEFRKRLPMY
FWVAGWETCRRMLAFLKDKENRRLALRTRLIRVKAVSKERSAEPRIRR
HRHAEIILSMPGFGVILGAEFLAATGGDMAAFASADRLAGVAGLAPVP
RDSGRISGNLKRPRRYDRRLLRACVSIRTDPSSRTYYDRKRTEGKRHT
QAVLALARRRLNVLWAMLRDHAVYHPATTTAAARLKLRRGERPMSLGQ
VFDPRANALHSFPLTGRMPWAPFIVSSWLRNPHPAQYFTARCLRILPG
LWIGAQGGSAAKLLMSGAPIEYVLKDSAVWMFKFDIGGTPRDIPVAGI
WNGSLWTPAWGGIHAIASNAYQFRNVIPARWSVSSAVLPNYRLVAALP
MAYHNQRMRFRTDLSYGVYGFAEINPIALVEKPALSWKSRLRRKNSSI
ALANMEDGGSVGRSNDIPGRRARFIGEKAEDPPAPSPRPALRIPNPLL
GLD;
(SEQ ID NO: 66)
MVINDDAQRLLSQRVDAGKADHYAVTTLADGGEVTWAIDLNAGGAALL
IALLIAAGQRLLYIPGATVHHAAGSYRGEAPVKQTLNFDLLKLAGDVE
SNPGPMQIFVKLPLLTIGDQFPAYELTALIAGDLSKVDAKQPGDYFTT
VTSEDHAGKWRVVFFWPKDFTGPEIATFGKLNDEFEDRDAQVLGVSID
SEFVHFNWRAQHEDLKNLPFPMLSDIKRELSLATGVLNADGVADRATF
IVDPNNEIQFVSVTAGSVGRNVEEVLRVLDALQSDELCACNWRKGDPT
LNATELLKASALGSIVGQTYREVEVVLVDGGSTDRTLDIANSFRPELG
SRLVVHSGPDDGPYDAMNRGVGVATGEWVLFLGADDTLYEPTTLAQVA
AFLGDHAASHLVYGDVVMRSTKSRHAGPFDLDRLLFETNLCHQSIFYR
RELFDGIGPYNLRYRVWADWDFNIRCFSNPALITRYMDVVISEYNDMT
GFSMRQGTDKEFRKRLPMYFWVAGWETCRRMLAFLKDKENRRLALRTR
LIRVKAVSKERSAEPRIRRHRHAEIILSMPGFGVILGAEFLAATGGDM
AAFASADRLAGVAGLAPVPRDSGRISGNLKRPRRYDRRLLRACYLSAL
VSIRTDPSSRTYYDRKRTEGKRHTQAVLALARRRLNVLWAMLRDHAVY
HPATTTAAARLKLRRGERPMSLGQVFDPRANALHSFPLTGRMPWAPFI
VSSWLRNPHPAQYFTARCLRILPGLWIGAQGGSAAKLLMSGAPIEYVL
KDSAVWMFKFDIGGTPRDIPVAGIWNGSLWTPAWGGIHAIASNAYQFR
NVIPARWSVSSAVLPNYRLVAALPMAYHNQRMRFRTDLSYGVYGFAEI
NPIALVEKPALSWKSRLRRKNSSIALANMEDGGSVGRSNDIPGRRARF
IGEKAEDPPAPSPRPALRIPNPLLGLD;
or
(SEQ ID NO: 67)
MVINDDAQRLLSQRVDAGKADHYAVTTLADGGEVTWAIDLNAGGAALL
IALLIAAGQRLLYIPGATVHHAAGSYRGEAPVKQTLNFDLLKLAGDVE
SNPGPMQIFVKLPLLTIGDQFPAYELTALIAGDLSKVDAKQPGDYFTT
VTSEDHAGKWRVVFFWPKDFTGPEIATFGKLNDEFEDRDAQVLGVSID
SEFVHFNWRAQHEDLKNLPFPMLSDIKRELSLATGVLNADGVADRATF
IVDPNNEIQFVSVTAGSVGRNVEEVLRVLDALQSDELCACNWRKGDPT
LNATELLKASALGSIVGQTYREVEVVLVDGGSTDRTLDIANSFRPELG
SRLVVHSGPDDGPYDAMNRGVGVATGEWVLFLGADDTLYEPTTLAQVA
AFLGDHAASHLVYGDVVMRSTKSRHAGPFDLDRLLFETNLCHQSIFYR
RELFDGIGPYNLRYRVWADWDFNIRCFSNPALITRYMDVVISEYNDMT
GFSMRQGTDKEFRKRLPMYFWVAGWETCRRMLAFLKDKENRRLALRTR
LIRVKAVSKERSAEPRIRRHRHAEIILSMPGFGVILGAEFLAATGGDM
AAFASADRLAGVAGLAPVPRDSGRISGNLKRPRRYDRRLLRACVSIRT
DPSSRTYYDRKRTEGKRHTQAVLALARRRLNVLWAMLRDHAVYHPATT
TAAARLKLRRGERPMSLGQVFDPRANALHSFPLTGRMPWAPFIVSSWL
RNPHPAQYFTARCLRILPGLWIGAQGGSAAKLLMSGAPIEYVLKDSAV
WMFKFDIGGTPRDIPVAGIWNGSLWTPAWGGIHAIASNAYQFRNVIPA
RWSVSSAVLPNYRLVAALPMAYHNQRMRFRTDLSYGVYGFAEINPIAL
VEKPALSWKSRLRRKNSSIALANMEDGGSVGRSNDIPGRRARFIGEKA
EDPPAPSPRPALRIPNPLLGLD
(SEQ ID NO: 68)
MVINDDAQRLLSQRVDAGKADHYAVTTLADGGEVTWAIDLNAGGAALL
IALLIAAGQRLLYIPGRTVHHAAGSYRGEAPVKQTLNFDLLKLAGDVE
SNPGPMQIFVKLPLLTIGDQFPAYELTALIAGDLSKVDAKQPGDYFTT
VTSEDHAGKWRVVFFWPKDFTGPEIATFGKLNDEFEDRDAQVLGVSID
SEFVHFNWRAQHEDLKNLPFPMLSDIKRELSLATGVLNADGVADRATF
IVDPNNEIQFVSVTAGSVGRNVEEVLRVLDALQSDELCACNWRKGDPT
LNATELLKASALGSIVGQTYREVEVVLVDGGSTDRTLDIANSFRPELG
SRLVVHSGPDDGPYDAMNRGVGVATGEWVLFLGADDTLYEPTTLAQVA
AFLGDHAASHLVYGDVVMRSTKSRHAGPFDLDRLLFETNLCHQSIFYR
RELFDGIGPYNLRYRVWADWDFNIRCFSNPALITRYMDVVISEYNDMT
GFSMRQGTDKEFRKRLPMYFWVAGWETCRRMLAFLKDKENRRLALRTR
LIRVKAVSKERSAEPRIRRHRHAEIILSMPGFGVILGAEFLAATGGDM
AAFASADRLAGVAGLAPVPRDSGRISGNLKRPRRYDRRLLRACYLSAL
VSIRTDPSSRTYYDRKRTEGKRHTQAVLALARRRLNVLWAMLRDHAVY
HPATTTAAARLKLRRGERPMSLGQVFDPRANALHSFPLTGRMPWAPFI
VSSWLRNPHPAQYFTARCLRILPGLWIGAQGGSAAKLLMSGAPIEYVL
KDSAVWMFKFDIGGTPRDIPVAGIWNGSLWTPAWGGIHAIASNAYQFR
NVIPARWSVSSAVLPNYRLVAALPMAYHNQRMRFRTDLSYGVYGFAEI
NPIALVEKPALSWKSRLRRKNSSIALANMEDGGSVGRSNDIPGRRARF
IGEKAEDPPAPSPRPALRIPNPLLGLD;
or
(SEQ ID NO: 69)
MVINDDAQRLLSQRVDAGKADHYAVTTLADGGEVTWAIDLNAGGAALL
IALLIAAGQRLLYIPGRTVHHAAGSYRGEAPVKQTLNFDLLKLAGDVE
SNPGPMQIFVKLPLLTIGDQFPAYELTALIAGDLSKVDAKQPGDYFTT
VTSEDHAGKWRVVFFWPKDFTGPEIATFGKLNDEFEDRDAQVLGVSID
SEFVHFNWRAQHEDLKNLPFPMLSDIKRELSLATGVLNADGVADRATF
IVDPNNEIQFVSVTAGSVGRNVEEVLRVLDALQSDELCACNWRKGDPT
LNATELLKASALGSIVGQTYREVEVVLVDGGSTDRTLDIANSFRPELG
SRLVVHSGPDDGPYDAMNRGVGVATGEWVLFLGADDTLYEPTTLAQVA
AFLGDHAASHLVYGDVVMRSTKSRHAGPFDLDRLLFETNLCHQSIFYR
RELFDGIGPYNLRYRVWADWDFNIRCFSNPALITRYMDVVISEYNDMT
GFSMRQGTDKEFRKRLPMYFWVAGWETCRRMLAFLKDKENRRLALRTR
LIRVKAVSKERSAEPRIRRHRHAEIILSMPGFGVILGAEFLAATGGDM
AAFASADRLAGVAGLAPVPRDSGRISGNLKRPRRYDRRLLRACVSIRT
DPSSRTYYDRKRTEGKRHTQAVLALARRRLNVLWAMLRDHAVYHPATT
TAAARLKLRRGERPMSLGQVFDPRANALHSFPLTGRMPWAPFIVSSWL
RNPHPAQYFTARCLRILPGLWIGAQGGSAAKLLMSGAPIEYVLKDSAV
WMFKFDIGGTPRDIPVAGIWNGSLWTPAWGGIHAIASNAYQFRNVIPA
RWSVSSAVLPNYRLVAALPMAYHNQRMRFRTDLSYGVYGFAEINPIAL
VEKPALSWKSRLRRKNSSIALANMEDGGSVGRSNDIPGRRARFIGEKA
EDPPAPSPRPALRIPNPLLGLD.

The vaccine may be a nucleic acid vaccine comprising a polynucleotide encoding any one or more of the polypeptides described herein. The vaccine may comprise a polynucleotide comprising two or more copies, such as 3, 4, 5 or more copies of a nucleotide sequence encoding any one or more of the peptides or polypeptides described herein.

### Peptides

The invention provides immunogenic MAP peptides. The invention provides a peptide of up to 100 amino acids comprising the sequence MVINDDAQRLLSQRVANDEAALLELI (SEQ ID NO: 4) or (SEQ ID NO: 6). The peptide of the invention may have a length of from 26 to 100, 30 to 95, 35 to 80, 40 to 75, 45 to 70, 50 to 65, 55 to 60 amino acids. The peptide may be a fragment of P900.

The peptide of the invention may further comprise the amino acid sequence: VTTLADGGEVTWAID or VTTLADGGEVTWAIDLNA; and/or VDAGKADHY, EVVVAQPVWAGVDAGKADHY or MTVTEVVVAQPVWAGVDAGKADHY. The peptide of the invention may comprise the amino acid sequence of any one of SEQ ID NOs: 11 to 20. The peptide linker in any one of SEQ ID NOs: 1 to 20 may be any of the peptide linkers as described below.

The peptide may comprise, consist of or consist essentially of the amino acid sequence of SEQ ID NO: 4 or SEQ ID NO: 6.

### Polypeptides

In one aspect, the invention provides a polypeptide that comprises an amino acid sequence of at least 9 contiguous amino acids from a region of MAP P900 fused to one or more additional MAP polypeptides, or a fragment thereof. The additional MAP polypeptide may, in one embodiment, be a fragment comprising the all or part of the C-terminal extracellular region of P900. Alternatively, or additionally, the at least one additional MAP polypeptide may be comprised of all or part of a different MAP protein.

The polypeptide comprising an amino acid sequence of at least 9 contiguous amino acids from the region of MAP P900 shown in SEQ ID NO: 2 may be any of the polypeptides described above. In preferred embodiments, this polypeptide comprises the amino acid sequence shown in SEQ ID NO: 3, the amino acid sequence shown in SEQ ID NO: 7 and/or the amino acid sequence shown in SEQ ID NO: 9. In a particularly preferred embodiment, this polypeptide comprises the amino acid sequence shown in SEQ ID NO: 3.

The p12 polypeptide disclosed herein comprises the C-terminal extracellular region of MAP P900. Therefore, in one embodiment, the additional MAP polypeptide is the p12 polypeptide or a fragment or variant thereof. Any one of the p12 polypeptides disclosed herein may be used. For example, the fragment of the p12 polypeptide may comprise, consist essentially of, or consist of the amino acid sequence shown in SEQ ID NO: 48 and/or or any one of SEQ ID NOs: 44 to 47.

The additional MAP polypeptide may be an ahpC polypeptide, a gsd polypeptide, a p12 polypeptide and/or a mpa polypeptide. In one embodiment, the polypeptide comprises the amino acid sequence MVINDDAQRLLSQR and any one of or any combination of an ahpC polypeptide, a gsd polypeptide, a p12 polypeptide and a mpa polypeptide. In some embodiments, the polypeptide may be phosphorylated at the serine residue in the MVINDDAQRLLSQR amino acid sequence.

The ahpC polypeptide can be any of the ahpC polypeptides described herein. The gsd polypeptide may be any of the gsd polypeptides described herein. The mpa polypeptide may be any of the MPA polypeptides described herein.

In a preferred embodiment, the N-terminal P900 polypeptide is provided with any one or more of the four polypeptides ahpC, gsd, p12 and mpa in a single fusion protein. The four polypeptide sequences in such a fusion protein may be any of the polypeptides or variants described herein. The four polypeptides may be provided in any order in the fusion protein. In one embodiment they are provided in the order N-terminal P900-ahpC-gsd-p12-mpa.

In one embodiment, the ahpC, gsd, p12 and mpa polypeptides present in a fusion protein are those given in SEQ ID NOs: 32, 34, 36 and 40.

In an alternative embodiment, the polypeptides may be present in two or more separate polypeptide molecules, which may or may not be linked by non-covalent linkages. For example, the polypeptides may be provided separately, or may be provided in two, three or more separate fusion protein polypeptide molecules. For example, the N-terminal P900 polypeptide may be provided as one molecule and the ahpC, gsd, p12 and mpa may be provided as a fusion protein.

In the fusion protein, linker sequences may separate the required polypeptide sequences and/or there may or may not be additional sequences present at the N terminal or C terminal of the peptide. Typically the fusion protein comprises 1, 2, 3, or more such linkers. The linkers are typically 1, 2, 3, 4 or more amino acids in length. Thus in the peptide 1, 2, 3 or all of the polypeptide sequences may be contiguous with each other or may be separated from each other, for example by such linkers.

The peptides and polypeptides may be used as induce immune responses against MAP in subjects.

In one embodiment the polypeptide comprises the amino acid sequence of SEQ ID NO: 41 wherein a peptide comprising an amino acid sequence of at least 9 contiguous amino acids from the region of MAP P900 shown in SEQ ID NO: 2, preferably the amino acid sequence MVINDDAQRLLSQR, is inserted between: positions 7 and 8, positions 199 and 200, positions 442 and 443, positions 577 and 578, and/or positions 820 and 821.

In another embodiment, the polypeptide comprises the amino acid sequence of SEQ ID NO: 42 wherein a peptide comprising an amino acid sequence of at least 9 contiguous amino acids from the region of MAP P900 shown in SEQ ID NO: 2, preferably the amino acid sequence MVINDDAQRLLSQR, is inserted between: positions 7 and 8, positions 199 and 200, positions 442 and 443, positions 582 and 583, and/or positions 825 and 826.

In either SEQ ID NO: 41 or 42, the MQIFVKL leader sequence may be replaced by any other suitable leader sequence. Similarly, the PALRIPNPLLGLD tag may be replaced by any other tag. The insertions of the at least 9 contiguous amino acids from SEQ ID NO: 2 may be made directly into the one or more of the indicated positions of SEQ ID NO: 41 or 42, or a peptide linker may be included at one or both ends of the inserted sequence.

### Variants

A “polypeptide” is used herein in its broadest sense to refer to a compound of two or more subunit amino acids, amino acid analogs, or other peptidomimetics. The term “polypeptide” is used synonymously with the term “peptide”. The term “polypeptide” thus includes short peptide sequences and also longer polypeptides and proteins. As used herein, the term “amino acid” refers to either natural and/or unnatural or synthetic amino acids, including glycine and both the D or L optical isomers, and amino acid analogs and peptidomimetics.

A variant of a polypeptide may comprise 1, 2, 3, 4, 5, up to 10, up to 20, up to 30 or more amino acid substitutions and/or deletions from the sequences given in the sequence listing. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

Preferred “variants” include those in which instead of the naturally occurring amino acid the amino acid which appears in the sequence is a structural analog thereof. Amino acids used in the sequences may also be derivatized or modified, e.g. labelled, providing the function of the peptide is not significantly adversely affected.

Variants as described above may be prepared during synthesis of the peptide or by post-production modification, or when the peptide is in recombinant form using the known techniques of site-directed mutagenesis, random mutagenesis, or enzymatic cleavage and/or ligation of nucleic acids.

Suitable variants may comprise sequences of naturally occurring polypeptides from mycobacteria other than MAP. For example, a variant ahpC polypeptide sequence may derive from a different mycobacterial strain to MAP. Such naturally occurring variants preferably maintain the ability to stimulate an immune response which is capable of acting against MAP. That is, the immune response to the variant polypeptide will react against MAP polypeptides as well as the variant polypeptide used.

Preferably variants according to the invention have an amino acid sequence which has more than 60%, or more than 70%, e.g. 75 or 80%, preferably more than 85%, e.g. more than 90 or 95% amino acid identity to, for example, SEQ ID NO: 2, 22, 32, 24, 34, 26, 36, 30 or 40, (according to the test described hereinafter). This level of amino acid identity may be seen across the full length of the sequence or over a part of the sequence, such as 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G, P, S, N, D, Q, E, K, R. Sequence identity at a particular residue is intended to include identical residues which have simply been derivatized.

Particular modifications can be made to any of the wild type MAP proteins sequences given in SEQ ID Nos: 2, 22, 32, 24, 34, 26, 36, 30 or 40. For example, modification can be made to try to improve the overall properties of the variant protein as an immunogen.

In one embodiment, a wild-type protein may be modified by deletion or substitution to remove an acylation site. Such an acylation site might affect the overall conformation of the protein. By omitting acylation sites, for example by excluding or substituting a cysteine residue, the presentation of effective epitopes within the protein may be optimised. For example, the wild type MAP gsd sequence given in SEQ ID NO: 24 includes a cysteine residue at position 22. In the variant of SEQ ID NO: 34, the amino acid sequence has been modified by truncation at the N-terminal such that this cysteine residue is no longer present. Similarly, the N-terminal fragment of P900 shown in SEQ ID NO: 22 comprises a cysteine residue at position 25. This cysteine is preferably deleted, or substituted by one or more amino acid, such as by a peptide linker. Such a modification may be made to a wild type protein or to any of the variant or fragment sequences, such as the codon optimised sequences, described herein.

In another embodiment, a wild type MAP protein may be modified to disable or remove potential cross-reacting epitopes. For example, where a polypeptide of the invention is intended for use in a human, the polypeptide sequence may be modified to disable or remove potential cross-reacting human epitopes, such as sequences which generate antibodies in human patients which may cross-react with similar sequences in human proteins. Modifications may thus be made to the MAP sequences to avoid such cross-reactivity but to maintain the ability to generate an anti-MAP immune response.

For example within the wild type MAP gsd sequence the lysine residues at positions 239 and 241 (see SEQ ID NO: 24) may each be substituted with asparagine. An equivalent substitution may be made in any of the variant or fragment gsd sequences described herein. For example in the variant sequence of SEQ ID NO: 34, the lysine residues at positions 216 and 218 may be replaced with asparagines. This may be achieved by modifying the nucleic acid sequence which encodes the gsd polypeptide. For example, in the gsd polynucleotide sequence of SEQ ID NO: 33, the AAG codons at positions 646 to 648 and 651 to 654 may be replaced by AAT. This maintains the optimised human codon usage of SEQ ID NO: 33 and further removes potentially cross-reacting human epitopes.

Similarly, modifications mat be made to the MAP ahpC sequence. In the wild-type ahpC sequence of SEQ ID NO: 22, the lysine at position 29 may be replaced with threonine and the proline at position 31 may be replaced with leucine. An equivalent substitution may be made in any of the variant or fragment ahpC sequences described herein. For example, in the modified variant sequence of SEQ ID NO: 32, the same substitutions may be made at the position 28 lysine and the position 30 proline. This may be achieved by modifying the nucleic acid sequence which encodes the ahpC polypeptide. For example, in the ahpC polynucleotide sequence of SEQ ID NO: 31, the AAA codon at positions 82 to 84 may be replaced by ACA and the CCC codon at positions 88 to 90 may be replaced by CTC. This maintains the optimised human codon usage of SEQ ID NO: 31 and further removes potentially cross-reacting human epitopes.

Similarly, modifications may be made to reduce the hydrophobicity of the protein and thus to help optimise the surface presentation of epitopes. For example, the wild-type mpa sequence of SEQ ID NO: 30 includes ten transmembrane regions. In order to reduce the hydrophobicity of the protein, one or more of these regions, or parts of these regions, may be omitted or substituted. For example, one, more or all of the transmembrane regions may be deleted. Such regions may be deleted totally or partially, optionally leaving none, one, two or more amino acid residues from the ends of the transmembrane sequence in the protein. Thus one modification may be the deletion or substitution of one or more hydrophobic amino acids. An example of this is seen in SEQ ID NO: 40 which is a variant of MAP mpa in which most of the transmembrane sequences have been deleted, leaving only one or two amino acids from the transmembrane regions in the variant polypeptide. Another example is deletion of the four N-terminal amino acid residues, MTVT, of SEQ ID NO: 22.

Polypeptide “fragments” may be made by truncation, e.g. by removal of one or more amino acids from the N and/or C-terminal ends of a polypeptide. Up to 10, up to 20, up to 30, up to 40 or more amino acids may be removed from the N and/or C terminal in this way. Fragments may also be generated by one or more internal deletions. For example, a variant of the invention may consist of or comprise two or more epitope regions from a full length polypeptide of the region in the absence of non-epitope amino acids. Preferably a fragment of an N-terminal P900, ahpC, gsd, p12 or mpa polypeptide comprises at least one epitope capable of inducing an immune response against the unmodified MAP polypeptide. Such fragments may be derived from a sequence of SEQ ID NO: 2, 22, 32, 24, 34, 26, 36, 30 or 40, or may be derived from a variant peptide as described herein. Preferably such fragments are between 8 and 150 residues in length, e.g. 8 to 50 or 8 to 30 residues. Alternatively, fragments of the invention may be longer sequences, for example comprising at least 50%, at least 60%, at least 70%, at least 80% or at least 90% of the full length polypeptide.

Preferably, a variant is a functional variant thereof. In particular, a variant polypeptide should retain the ability to stimulate an immune response against the unmodified MAP polypeptide. In one embodiment, a functional variant polypeptide should be capable of acting as an antigen and should include at least one functional epitope from the original polypeptide.

An “antigen” refers to any agent, generally a macromolecule, which can elicit an immunological response in an individual. As used herein, “antigen” is generally used to refer to a polypeptide molecule or portion thereof which contains one or more epitopes. Furthermore, for the purposes of the present invention, an “antigen” includes a polypeptide having modifications, such as deletions, additions and substitutions (generally conservative in nature) to the native sequence, so long as the polypeptide maintains sufficient immunogenicity. These modifications may be deliberate, for example through site-directed mutagenesis, or may be accidental, such as through mutations of hosts which produce the antigens.

An “immune response” against an antigen of interest is the development in an individual of a humoral and/or a cellular immune response to that antigen. A “humoral immune response” refers to an immune response mediated by antibody molecules, while a “cellular immune response” is one mediated by T-lymphocytes and/or other white blood cells.

As used herein, the term “epitope” generally refers to the site on a target antigen which is recognised by an immune receptor such as a T-cell receptor and/or an antibody. Preferably it is a short peptide derived from or as part of a protein. However the term is also intended to include peptides with glycopeptides and carbohydrate epitopes. A single antigenic molecule may comprise several different epitopes. The term “epitope” also includes modified sequences of amino acids or carbohydrates which stimulate responses which recognise the whole organism.

It is advantageous if the selected epitope is specific to MAP, or involved in the pathogenicity of MAP. For example, it is advantageous if the immune receptor and/or antibody which recognises the epitope will only recognise this epitope from MAP, and not epitopes in other unrelated proteins, in particular proteins from unrelated organisms or host proteins. If the epitope is involved in pathogenicity of MAP, then an immune response against such an epitope may be used to target pathogenic MAP infections.

An epitope may also be related to equivalent epitopes on other mycobacteria. For example, many individuals suffering from MAP infection are also infected by M. avium as a secondary co-pathogen. Other M. avium complexes may be present or involved in Crohn's disease, Johne's disease, Ulcerative Colitis, Psoriasis, Thyroiditis, Sarcoidosis, Parkinson's disease, Multiple Sclerosis, Type 1 Diabetes, arthritis, ankylosing spondylitis, rheumatoid arthritis, irritable bowel syndrome, inflammatory bowel disease, chronic enteritis, Alzheimer's disease, multiple sclerosis, idiopathic pulmonary fibrosis, leprosy and/or chronic fatigue syndrome. Many of the proteins expressed in MAP such as AhpC are very similar to those expressed in M. avium. If the polypeptide of the invention includes one or more epitopes which are capable of stimulating an immune response which acts against M. avium in addition to MAP, a further, secondary, therapeutic effect may be achieved.

Epitopes can be identified from knowledge of the amino acid and corresponding DNA sequences of the peptide or polypeptide, as well as from the nature of particular amino acids (e.g., size, charge, etc.) and the codon dictionary, without undue experimentation. See, e.g., Ivan Roitt, Essential Immunology, 1988; Janis Kuby, Immunology, 1992 e.g., pp. 79-81. Some guidelines in determining whether a protein or an epitope of interest will stimulate a response, include: peptide length—the peptide should be at least 8 or 9 amino acids long to fit into the MHC class I complex and at least 8-25, such at least as 13-25 amino acids long to fit into a class II MHC complex. These lengths are the minimum for the peptide to bind to the respective MHC complex. It is preferred for the peptides to be longer than these lengths because cells may cut peptides. The peptide should contain an appropriate anchor motif which will enable it to bind to the various class I or class II molecules with high enough specificity to generate an immune response. This can be done, without undue experimentation, by comparing the sequence of the protein of interest with published structures of peptides associated with the MHC molecules. Thus, the skilled artisan can ascertain an epitope of interest by comparing the protein sequence with sequences listed in the protein database.

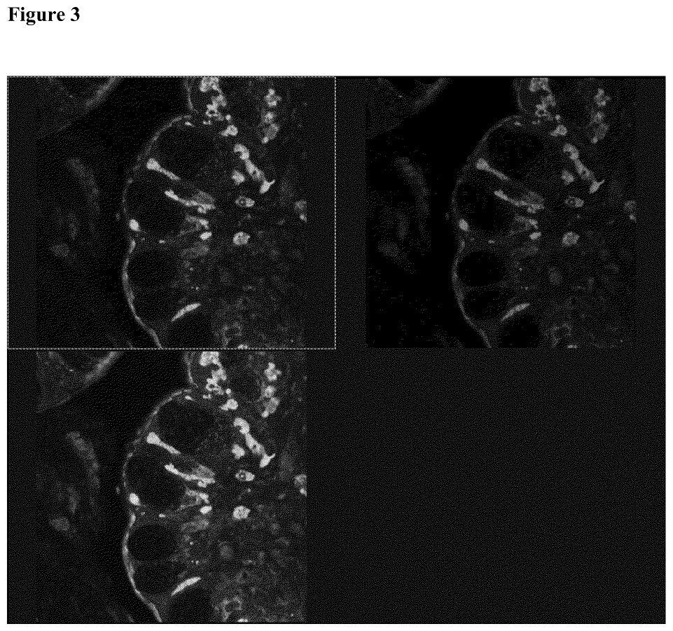

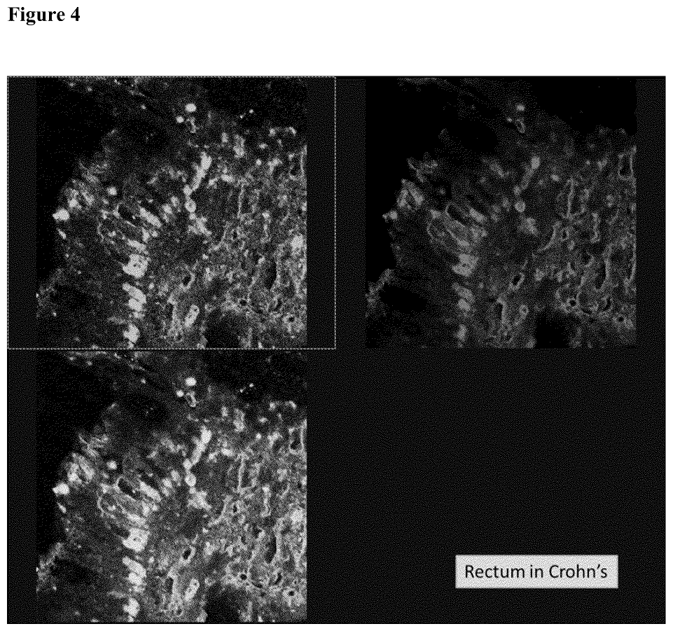

Suitable epitopes may thus be identified by routinely used methods such as those demonstrated in FIGS. 3 and 4 for identifying the strong T cell epitope GFAEINPIA (peptide 9.1) in the 5^{th} extracellular loop of mpa. In such a method, a library of short peptides which are fragments of the polypeptide sequence of interested may be generated and each of these peptides assessed separately for their ability to identify an immune response against the full length polypeptide. Members of the library may be screened in groups or pools or individual members of the library, such as individual members of a single pool, may be assessed separately.

In a further example, epitope scanning of the individual proteins of SEQ ID NOs: 32, 34, 36 and 40 revealed a number of predicted class I and class II epitopes.

In the ahpC variant sequence of SEQ ID NO: 32, predicted strong class II epitopes were identified at amino acids 48 to 56, 90 to 101 and 161 to 169. An ahpC polypeptide of the invention, such as an ahpC variant or fragment polypeptide, preferably comprises at least one, for example one, two or all three of these epitopes.

In the gsd variant sequence of SEQ ID NO: 34, predicted class I epitopes were identified at amino acids 1 to 32, 58 to 68, 99 to 119, 123 to 147, 159 to 169, 180 to 194 and 200 to 231, and predicted strong class II epitopes were identified at amino acids 64 to 76, 95 to 110, 192 to 206 and 223 to 240. A gsd polypeptide of the invention, such as a gsd variant or fragment polypeptide, preferably comprises at least one, for example one, two, three, four, five, six, seven, eight, nine, ten or all of these epitopes.

In the p12 variant sequence of SEQ ID NO: 36, predicted class I epitopes were identified at amino acids 33 to 56 and 98 to 117 and a predicted strong class II epitope was identified at amino acids 3 to 10. A p12 polypeptide of the invention, such as a p12 variant or fragment polypeptide, preferably comprises at least one, for example one, two or all three of these epitopes.

In the mpa variant sequence of SEQ ID NO: 40, a predicted class I epitope was identified at amino acids 130 to 160, and predicted strong class II epitopes were identified at amino acids 56 to 64 and 150 to 160. An mpa polypeptide of the invention, such as an mpa variant or fragment polypeptide, preferably comprises at least one, for example one, two or all three of these epitopes.

As shown in the Examples, a particular strong T cell epitope has been identified in the mpa polypeptide sequence. This epitope has the amino acid sequence GFAEINPIA and is located at amino acids 357 to 365 of SEQ ID NO: 30 and amino acids 177 to 185 of SEQ ID NO: 40. This sequence is found in the construct of SEQ ID NO: 42 at amino acids 761 to 769. A preferred mpa polypeptide sequence is a sequence which comprises GFAEINPIA. Such a sequence may also comprise one, two or all three of the predicted class I and class II epitopes mentioned above.

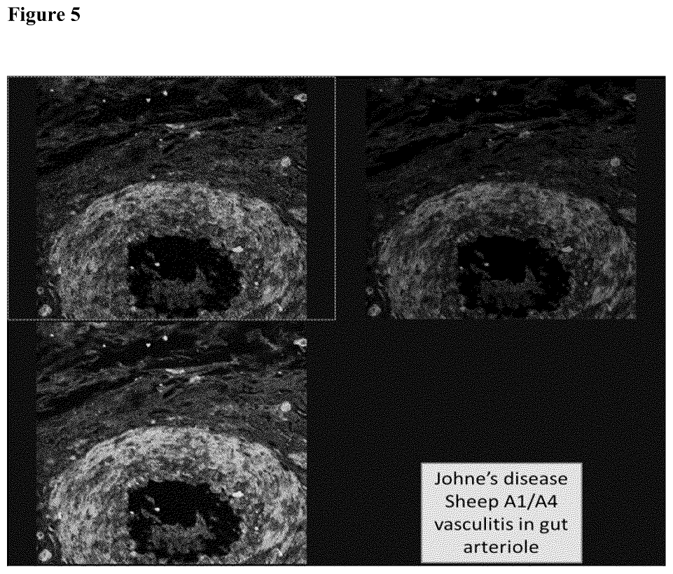

This epitope is believed to be located in the fifth extracellular loop of mpa (FIG. 5A). A preferred mpa polypeptide may therefore maintain the sequence of the fifth extracellular loop. An mpa polypeptide may therefore comprise the amino acid sequence GFAEINPIA and also adjacent amino acids from the fifth extracellular loop of mpa. Preferably, this fifth extracellular loop will be present in a polypeptide of the invention in a suitable form and conformation for it to be recognised by the immune system.

A peptide or polypeptide of the invention or for use in the invention may comprise further additional sequences, for example those encoded by the polynucleotides and vectors described below. For example, it may comprise additional epitopes, therapeutic polypeptides, adjuvants or immunomodulatory molecules.

The polypeptide may comprise a leader sequence, i.e. a sequence at or near the amino terminus of the polypeptide that functions in targeting or regulation of the polypeptide. For example a sequence may be included in the polypeptide that targets it to particular tissues in the body, or which helps the processing or folding of the polypeptide upon expression. Various such sequences are well known in the art and could be selected by the skilled reader depending upon, for example, the desired properties and production method of the polypeptide.

A polypeptide may further comprise a tag or label to identify or screen for the polypeptide, or for expression of the polypeptide. Suitable labels include radioisotopes such as ¹²⁵I, ³²P or ³⁵S, fluorescent labels, enzyme labels, or other protein labels such as biotin. Suitable tags may be short amino acid sequences that can be identified by routine screening methods. For example, a short amino acid sequence may be included that is recognised by a particular monoclonal antibody.

The sequence given in SEQ ID NO: 40 or 41 comprises the four modified polypeptides of SEQ ID NOs: 32, 34, 36 or 37, and 40, and additional sequences such as a ubiquitin leader sequence and a pK tag.

Peptides of the invention, as defined herein, may be chemically modified, for example, post-translationally modified. For example they may be glycosylated or comprise modified amino acid residues. They can be in a variety of forms of polypeptide derivatives, including amides and conjugates with polypeptides.

Chemically modified peptides also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Peptides may also be modified by phosphorylation, for example 3 amino phosphorylation and by glycosylation for example mannosylation.

Also included as chemically modified peptides are those which contain one or more naturally occurring amino acid derivatives of the twenty standard amino acids. For example, 4-hydroxyproline may be substituted for proline or homoserine may be substituted for serine.

The peptide of the invention or polypeptide or the polypeptides in the vaccine of the invention may be modified at the N-terminus and/or at the C-terminus and/or may be conjugated or coupled to a carrier molecule. Peptides/polypeptides may, for example, be conjugated to a bacterial saccharide or a carrier protein, such as keyhole limpet hemocyanin (KLH), bovine serum albumin (BSA), human serum albumin (HSA) or ovalbumin (OVA). The peptides may be biotinylated at the N-terminus or C-terminus, may be amidated at the N-terminus or C-terminus and/or may have a peptide tag added at the N-terminus or the C-terminus. The peptide tag may be, for example, a polylysine, such as a branched polylysine octamer, or a cell penetrating peptide such as an oligo-arginine (e.g. a polyarginine octamer or nonomer). Preferably, the peptide is biotinylated at the N-terminus and has an amide group or a branched polylysine octamer at the C-terminus. One or more additional amino acid residues may be added at the N-terminus and/or the C-terminus, optionally in addition to other terminal modifications. For example, one or more, such as two, alanine residues may be added at the N-terminus to increase immunogenicity and specificity and/or charged residues, for example GKK may be added at the N-terminus or preferably the C-terminus to reduce hydrophobicity. Where residues, such as GKK, are added at one terminus, the mirror image residues, such as KKG, may be added at the other terminus.

### Peptide Linkers

The peptides and polypeptides of the invention may comprise two or more, such as 3, 4, 5, 6 or more fragments of MAP polypeptides joined together by one or more peptide linkers. The peptide linker may, for example, be any suitable multi-epitope vaccine linker. The linker may be, for example, from 1 to 15 amino acids in length, such as from 2 to 10, 3 to 6, or 4 to 5 amino acids in length. Specific examples of suitable linkers include: GGG, GG, SGSG, AG, GGGS, AAY, a dilysine linker (KK) EAAAK, AAY and HEYGAEALERAG.

### Polynucleotides

The invention also relates to polynucleotide constructs comprising nucleic acid sequences which encode a peptide or polypeptide of the invention. For example, a single nucleic acid molecule may be provided which encodes any of the peptides or polypeptides, such as the fusion proteins, described above. The vaccine of the invention may comprise any one or more of the polynucleotides described herein.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

In one embodiment, therefore, a polynucleotide of the invention comprises a nucleic acid sequence comprising all or part of any one of the sequences shown in SEQ ID NOs: 21, 31, 23, 33, 25, 35, 29, 39, 41, 62, 63, 64, 65, 67, 68 and 69. The nucleic acid sequence in the polynucleotide may alternatively be a variant of one of these specific sequences. For example, a variant may be a substitution, deletion or addition variant of any of the above nucleic acid sequences. A variant of one of the four genes may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to one of the MAP polynucleotides shown in SEQ ID NOs: 1, 21, 23, 25, 27, 29, 31, 33, 35, 37, 39, 41, 62, 63, 64, 65, 67, 68 and 69 preferably at least 80 or 90% and more preferably at least 95%, 97% or 99% homologous thereto. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified MAP polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologues typically hybridize with the relevant polynucleotide at a level significantly above background. The signal level generated by the interaction between the homologue and the polynucleotide is typically at least 10 fold, preferably at least 100 fold, as intense as “background hybridisation”. The intensity of interaction may be measured, for example, by radiolabelling the probe, e.g. with ³²P. Selective hybridisation is typically achieved using conditions of medium to high stringency, (for example, 0.03M sodium chloride and 0.003M sodium citrate at from about 50° C. to about 60° C.

Stringent hybridization conditions can include 50% formamide, 5× Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

In one embodiment the coding sequence of the polynucleotide construct may be optimised to more closely resemble the codon usage of highly expressed genes in mammalian cells. Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria and mammalian cells, and some species show a stronger bias away from a random codon selection than others.

For example, humans and other mammals are less strongly biased than certain bacteria or viruses. For these reasons, it is possible that, for example a mycobacterial gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In the polynucleotide of the invention, the codon usage pattern may therefore be altered from that found naturally in MAP to more closely represent the codon bias of the target organism, e.g. a mammal, especially a human. The “codon usage coefficient” is a measure of how closely the codon pattern of a given polynucleotide sequence resembles that of a target species. Codon frequencies can be derived from literature sources for the highly expressed genes of many species (see e.g. Nakamura et. al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are “frequently used codons”. If a polynucleotide sequence has a codon usage coefficient of 1, all of the codons are “most frequent” codons for highly expressed genes of the target species.

According to the present invention, the codon usage pattern of the polynucleotide of the invention will preferably exclude codons with a relative synonymous codon usage (RSCU) value of less than 0.2 in highly expressed genes of the target organism. A RSCU value is the observed number of codons divided by the number expected if all codons for that amino acid were used equally frequently. The polynucleotide of the invention will generally have a codon usage coefficient for highly expressed human genes of greater than 0.3, preferably greater than 0.4, most preferably greater than 0.5. Codon usage tables for human can also be found in GenBank.

It can thus be seen that the particular polynucleotide sequence which encodes a polypeptide of the invention may be altered to optimise the codons based on the species to be treated. As an example of this, the MAP sequences given in SEQ ID Nos: 21, 23, 25 and 29 have been codon optimised for human use in the polynucleotides of SEQ ID Nos: 31, 33, and 35. Such modifications may improve the ability of such polynucleotides to express their encoded proteins in a human cell.

As explained above in relation to polypeptides, the polynucleotides of the invention may also be modified to disable or remove potential cross-reacting epitopes in the encoded polypeptide.

Polynucleotide “fragments” according to the invention may be made by truncation, e.g. by removal of one or more nucleotides from one or both ends of a polynucleotide. Up to 10, up to 20, up to 30, up to 40, up to 50, up to 75, up to 100, up to 200 or more amino acids may be removed from the 3′ and/or 5′ end of the polynucleotide in this way. Fragments may also be generated by one or more internal deletions. For example, a variant of the invention may encode a polypeptide that consists of or comprises two or more epitope regions from a full length polypeptide of the invention in the absence of non-epitope amino acids. Preferably a fragment of an ahpC, gsd, p12 or mpa polynucleotide sequence comprises at least one region encoding an epitope capable of inducing an immune response against the unmodified MAP polypeptide. Such fragments may be derived from a sequence of SEQ ID NO: 21, 31, 23, 33, 25, 35, 29 or 39 or may be derived from a variant polynucleotide as described herein. Preferably such fragments are between 24 and 500 residues in length, e.g. 24 to 400, 24 to 300, 24 to 100, 100 to 200 or 200 to 400 residues. Alternatively, fragments of the invention may be longer sequences, for example comprising at least 50%, at least 60%, at least 70%, at least 80% or at least 90% of a full length polynucleotide of the invention.

A peptide of the invention may thus be produced from or delivered in the form of a polynucleotide which encodes, and is capable of expressing, it. Polynucleotides of the invention can be synthesised according to methods well known in the art, as described by way of example in Sambrook et al (1989, Molecular Cloning—a laboratory manual; Cold Spring Harbor Press). Substantially pure antigen preparations can be obtained using standard molecular biological tools. That is, polynucleotide sequences coding for the above-described moieties can be obtained using recombinant methods, such as by screening cDNA and genomic libraries from cells expressing an antigen, or by deriving the coding sequence for a polypeptide from a vector known to include the same. Furthermore, the desired sequences can be isolated directly from cells and tissues containing the same, using standard techniques, such as phenol extraction and PCR of cDNA or genomic DNA. See, e.g., Sambrook et al., supra, for a description of techniques used to obtain and isolate DNA. Polynucleotide sequences can also be produced synthetically, rather than cloned.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used.

Once the sequences have been obtained, they may be linked together to provide a nucleic acid molecule using standard cloning or molecular biology techniques. Alternatively, the sequences can be produced synthetically, rather than cloned. The nucleotide sequence can be designed with the appropriate codons for the particular amino acid sequence desired. As explained herein, one will generally select preferred codons for the intended host in which the sequence will be expressed. The complete sequence can then be assembled from overlapping oligonucleotides prepared by standard methods and assembled into a complete coding sequence.

### Vectors

The nucleic acid molecules of the present invention may be provided in the form of an expression cassette which includes control sequences operably linked to the inserted sequence, thus allowing for expression of the polypeptide of the invention in vivo in a targeted subject species. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

Thus, a polypeptide of the invention may be provided by delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extrachromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters, such as β-actin promoters, may be used. Tissue-specific promoters are especially preferred. Mammalian promoters include the metallothionein promoter which can be induced in response to heavy metals such as cadmium.

In one embodiment a viral promoter is used to drive expression from the polynucleotide. Typical viral promoters for mammalian cell expression include the SV40 large T antigen promoter, adenovirus promoters, the Moloney murine leukaemia virus long terminal repeat (MMLV LTR), the mouse mammary tumor virus LTR promoter, the rous sarcoma virus (RSV) LTR promoter, the SV40 early promoter, the human cytomegalovirus (CMV) IE promoter, adenovirus, including the adenovirus major late promoter (Ad MLP), HSV promoters (such as the HSV IE promoters), or HPV promoters, particularly the HPV upstream regulatory region (URR). All these promoters are readily available in the art.

In one embodiment, the promoter is a Cytomegalovirus (CMV) promoter. A preferred promoter element is the CMV immediate early (IE) promoter devoid of intron A, but including exon 1. Thus the expression from the polynucleotide may be under the control of hCMV IE early promoter. Expression vectors using the hCMV immediate early promoter include for example, pWRG7128, and pBC12/CMV and pJW4303. A hCMV immediate early promoter sequence can be obtained using known methods. A native hCMV immediate early promoter can be isolated directly from a sample of the virus, using standard techniques. U.S. Pat. No. 5,385,839, for example, describes the cloning of a hCMV promoter region. The sequence of a hCMV immediate early promoter is available at Genbank #M60321 (hCMV Towne strain) and X17403 (hCMV Ad169 strain). A native sequence could therefore be isolated by PCR using PCR primers based on the known sequence. See e.g. Sambrook et al, supra, for a description of techniques used to obtain and isolate DNA. A suitable hCMV promoter sequence could also be isolated from an existing plasmid vector. Promoter sequences can also be produced synthetically.

A polynucleotide, expression cassette or vector of the invention may comprise an untranslated leader sequence. In general the untranslated leader sequence has a length of from about 10 to about 200 nucleotides, for example from about 15 to 150 nucleotides, preferably 15 to about 130 nucleotides. Leader sequences comprising, for example, 15, 50, 75 or 100 nucleotides may be used. Generally a functional untranslated leader sequence is one which is able to provide a translational start site for expression of a coding sequence in operable linkage with the leader sequence.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the expression cassette or vector.

Expression systems often include transcriptional modulator elements, referred to as “enhancers”. Enhancers are broadly defined as a cis-acting agent, which when operably linked to a promoter/gene sequence, will increase transcription of that gene sequence. Enhancers can function from positions that are much further away from a sequence of interest than other expression control elements (e.g. promoters), and may operate when positioned in either orientation relative to the sequence of interest. Enhancers have been identified from a number of viral sources, including polyoma virus, BK virus, cytomegalovirus (CMV), adenovirus, simian virus 40 (SV40), Moloney sarcoma virus, bovine papilloma virus and Rous sarcoma virus. Examples of suitable enhancers include the SV40 early gene enhancer, the enhancer/promoter derived from the long terminal repeat (LTR) of the Rous Sarcoma Virus, and elements derived from human or murine CMV, for example, elements included in the CMV intron A sequence.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus.

Nucleic acids encoding for polypeptides known to display antiviral or antibacterial activity, immunomodulatory molecules such as cytokines (e.g. TNF-alpha, interferons such as IL-6, and IL-2, interferons, colony stimulating factors such as GM-CSF), adjuvants and co-stimulatory and accessory molecules (B7-1, B7-2) may be included in a polynucleotide, expression cassette or vector of the invention. Alternatively, such polypeptides may be provided separately, for example in a formulation comprising a molecule of the invention, or may be administered simultaneously, sequentially or separately with a composition of the invention. Concurrent provision of an immunomodulatory molecule and a polypeptide of the invention at a site in vivo may enhance the generation of specific effectors which may help to enhance the immune response. The degree of enhancement of the immune response may be dependent upon the specific immunostimulatory molecules and/or adjuvants used because different immunostimulatory molecules may elicit different mechanisms for enhancing and/or modulating the immune response. By way of example, the different effector mechanisms/immunomodulatory molecules include but are not limited to augmentation of help signal (IL-2), recruitment of professional APC (GM-CSF), increase in T cell frequency (IL-2), effect on antigen processing pathway and MHC expression (IFN-gamma and TNF-alpha) and diversion of immune response away from the Th1 response and towards a Th2 response. Unmethylated CpG containing oligonucleotides are also preferential inducers of a Th1 response and are suitable for use in the present invention.

In some embodiments, the polynucleotide, expression cassette or vector will encode an adjuvant, or an adjuvant will otherwise be provided. As used herein, the term “adjuvant” refers to any material or composition capable of specifically or non-specifically altering, enhancing, directing, redirecting, potentiating or initiating an antigen-specific immune response.

A suitable adjuvant may be an ADP-ribosylating bacterial toxin. These include diphtheria toxin (DT), pertussis toxin (PT), cholera toxin (CT), the E. coli heat labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile and Staphylococcus aureus EDIN. Most ADP-ribosylating bacterial toxins contain A and B subunits.

Polynucleotides of interest may be used in vitro or in vivo in the production of a peptide of the invention. Such polynucleotides may be administered or used in the manufacture of a medicament for the treatment of Crohn's disease or another disease or condition characterised by the expression of MAP.

Gene therapy and nucleic acid immunization are approaches which provide for the introduction of a nucleic acid molecule encoding one or more selected antigens into a host cell for the in vivo expression of the antigen or antigens. Methods for gene delivery are known in the art. See, e.g., U.S. Pat. Nos. 5,399,346, 5,580,859 and 5,589,466. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.”

Each of these delivery techniques requires efficient expression of the nucleic acid in the transfected cell, to provide a sufficient amount of the therapeutic or antigenic gene product. Several factors are known to affect the levels of expression obtained, including transfection efficiency, and the efficiency with which the gene or sequence of interest is transcribed and the mRNA translated.

The agent produced by a host cell may be secreted or may be contained intracellularly depending on the polynucleotide and/or the vector used. As will be understood by those of skill in the art, expression vectors containing the polynucleotides of the invention can be designed with signal sequences which direct secretion of the polypeptide expressed from the vector through a particular prokaryotic or eukaryotic cell membrane.

The vectors and expression cassettes of the present invention may be administered directly as “a naked nucleic acid construct”, preferably further comprising flanking sequences homologous to the host cell genome. As used herein, the term “naked DNA” refers to a vector such as a plasmid comprising a polynucleotide of the present invention together with a short promoter region to control its production. It is called “naked” DNA because the vectors are not carried in any delivery vehicle. When such a vector enters a host cell, such as a eukaryotic cell, the proteins it encodes are transcribed and translated within the cell.

The vector of the invention may thus be a plasmid vector, that is, an autonomously replicating, extrachromosomal circular or linear DNA molecule. The plasmid may include additional elements, such as an origin of replication, or selector genes. Such elements are known in the art and can be included using standard techniques. Numerous suitable expression plasmids are known in the art. For example, one suitable plasmid is pSG2. This plasmid was originally isolated from Streptomyces ghanaensis. The length of 13.8 kb, single restriction sites for HindIII, EcoRV and PvuII and the possibility of deleting non-essential regions of the plasmid make pSG2 a suitable basic replicon for vector development.

Alternatively, the vectors of the present invention may be introduced into suitable host cells using a variety of viral techniques which are known in the art, such as for example infection with recombinant viral vectors such as retroviruses, herpes simplex viruses and adenoviruses.

In one embodiment, the vector itself may be a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors. In the case of viral vectors, administration of the polynucleotide is mediated by viral infection of a target cell.

A number of viral based systems have been developed for transfecting mammalian cells.

For example, a selected recombinant nucleic acid molecule can be inserted into a vector and packaged as retroviral particles using techniques known in the art. The recombinant virus can then be isolated and delivered to cells of the subject either in vivo or ex vivo. Retroviral vectors may be based upon the Moloney murine leukaemia virus (Mo-MLV). In a retroviral vector, one or more of the viral genes (gag, pol & env) are generally replaced with the gene of interest.

A number of adenovirus vectors are known. Adenovirus subgroup C serotypes 2 and 5 are commonly used as vectors. The wild type adenovirus genome is approximately 35 kb of which up to 30 kb can be replaced with foreign DNA. There are four early transcriptional units (E1, E2, E3 & E4), which have regulatory functions, & a late transcript, which codes for structural proteins. Adenovirus vectors may have the E1 and/or E3 gene inactivated. The missing gene(s) may then be supplied in trans either by a helper virus, plasmid or integrated into a helper cell genome. Adenovirus vectors may use an E2a temperature sensitive mutant or an E4 deletion. Minimal adenovirus vectors may contain only the inverted terminal repeats (ITRs) & a packaging sequence around the transgene, all the necessary viral genes being provided in trans by a helper virus. Suitable adenoviral vectors thus include Ad5 vectors and simian adenovirus vectors. For example, ChAdOx2, which is a is a simian-derived non-replicative vaccine vector developed by Oxford University for use in humans minimising cross reactivity to any pre-existing adenoviral immunity (Morris et al. Future Virol. 2016; 11(9):649-659) may be used. In some embodiments, a polynucleotide encoding any one of the above-described polypeptides, such as for example a polypeptide having the sequence shown in any one of SEQ ID NOs: 41 and 62 to 69, may by inserted into a ChAdOx2 vector in the same manner as the HAV ChAdOx2 vaccine construct containing HAV and expressing the MAP genes AhpC, Gsd, p12 and mpa described in (Morris et al. Future Virol. 2016; 11(9):649-659).

Viral vectors may also be derived from the pox family of viruses, including vaccinia viruses and avian poxvirus such as fowlpox vaccines. For example, modified vaccinia virus Ankara (MVA) is a strain of vaccinia virus which does not replicate in most cell types, including normal human tissues. A recombinant MVA vector may therefore be used to deliver the polypeptide of the invention.

Addition types of virus such as adeno-associated virus (AAV) or herpes simplex virus (HSV) may also be used to develop suitable vector systems.

As an alternative to viral vectors, liposomal preparations can alternatively be used to deliver the nucleic acid molecules of the invention. Useful liposomal preparations include cationic (positively charged), anionic (negatively charged) and neutral preparations, with cationic liposomes particularly preferred. Cationic liposomes may mediate intracellular delivery of plasmid DNA and mRNA.

As another alternative to viral vector systems, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyornithine, spermine, spermidine, as well as conjugates of these molecules.

In one embodiment, the vector may be a targeted vector, that is a vector whose ability to infect or transfect or transduce a cell or to be expressed in a host and/or target cell is restricted to certain cell types within the host subject, usually cells having a common or similar phenotype.

In one embodiment, the vector of the invention may comprise a single expression cassette, from which a single polypeptide sequence can be expressed. Alternatively, a vector of the invention may comprise two or more expression cassettes each capable of expressing a different polypeptide, such that the vector as a whole is capable of expressing all required polypeptides. Where the polypeptides are expressed from more than one locus in the vector, or are expressed as multiple separate molecules, the expression of the multiple sequences is preferably coordinated such that all polypeptides are expressed together. For example, the same or similar promoters may be used to control expression of the various components. Inducible promoters may be used so that expression of the various polypeptide components can be coordinated.

### Cell Lines

The invention also includes cells that have been modified to express a peptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a peptide of the invention include mammalian HEK293T, CHO, HeLa and COS cells. Preferably the cell line selected will be one which is not only stable, but also allows for mature glycosylation and cell surface expression of a polypeptide. Expression may be achieved in transformed oocytes. A suitable peptide may be expressed in cells of a transgenic non-human animal, preferably a mouse. A transgenic non-human animal expressing a peptide of the invention is included within the scope of the invention. A peptide of the invention may also be expressed in Xenopus laevis oocytes or melanophores.

Such cell lines of the invention may be cultured using routine methods to produce a polypeptide of the invention, or may be used therapeutically or prophylactically to deliver polypeptides of the invention to a subject. For example, cell lines capable of secreting a polypeptide of the invention may be administered to a subject. Alternatively, polynucleotides, expression cassettes or vectors of the invention may be administered to a cell from a subject ex vivo and the cell then returned to the body of the subject.

For example, methods for the ex vivo delivery and reimplantation of transformed cells into a subject are known (e.g., dextran-mediated transfection, calcium phosphate precipitation, electroporation, and direct microinjection into nuclei).

### Pharmaceutical Compositions

Formulation of a composition comprising a peptide, polypeptide, polynucleotide or vector as described above, can be carried out using standard pharmaceutical formulation chemistries and methodologies all of which are readily available to the reasonably skilled artisan. For example, compositions containing one or more molecules of the invention can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e. g., sterile pyrogen-free water) prior to parenteral administration of the reconstituted composition. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated compositions will include an amount of the molecule (e.g. vector) of interest which is sufficient to mount an immunological response. An appropriate effective amount can be readily determined by one of skill in the art. Such an amount will fall in a relatively broad range that can be determined through routine trials. The compositions may contain from about 0.1% to about 99.9% of the vector and can be administered directly to the subject or, alternatively, delivered ex vivo, to cells derived from the subject, using methods known to those skilled in the art.

The vaccine may in one aspect be peptide vaccine. In one embodiment the peptide(s) in the vaccine may be delivered using any suitable delivery system, for example an emulsion based delivery system, a liposome based delivery system, a virosome based delivery system, a transfersome based delivery system, an archeosome based delivery system, a niosome based delivery system, a cochleate based delivery system and/or a particulate delivery system.

The peptide may be administered with an adjuvant, or the vaccine may include an adjuvant. The adjuvant may, for example, be selected from Freund's complete adjuvant (CFA or FCA), Freund's incomplete adjuvant (IFA or FIA), Montanide™ ISA 720, ISCOMs, ISCOMATRIX™. Particulate delivery systems can also serve as adjuvants. The particulate delivery system may comprise nanoparticles. The nanoparticles may, for example be made of a natural polymer such as albumin, collagen, starch, chitosan or dextran, or of synthetic polymer such as polylactides (PLA), polyglycolides or polyglocolic acid (PGA) and their copolymers poly(lactide-co-glycolide) PLGA, poly(e-caprolactone) (PCL), poly(hydroxybutyrate) (PHB) and their copolymers. The nanoparticles may, alternatively, be carbon nanotubes, silicon dioxide nanoparticles, dendrimers, ferritin nanoparticles, peptide nanocarriers, gold nanoparticles, liposome-polycation-DNA (LPD) complex, oligosaccharide ester derivatives (OEDs) microparticles and combination systems.

### Therapeutic Methods

The vaccine, peptides, polypeptides, polynucleotides and vectors disclosed herein can be used in the treatment or prevention of infection by MAP, or in the treatment or prevention of any disease, condition or symptom which is associated with MAP infection, that is any disease condition or symptom which is a direct or indirect result of MAP infection, or which results from a disease or condition to which the presence of MAP contributes. MAP is known to be linked to numerous specific medical conditions, such as chronic inflammation of the intestine, including inflammatory bowel disease and as well as Irritable Bowel Syndrome. For example, MAP infection can cause chronic enteritis, such as Johne's disease (paratuberculosis) in livestock and Crohn's disease and Irritable Bowel Syndrome in humans. Other diseases or conditions associated with MAP include, but are not limited to, Parkinson's disease, Alzheimer's disease, Multiple Sclerosis, Irritable Bowel Syndrome, Ulcerative colitis, type 1 Diabetes Mellitus, Thyroiditis, Rheumatoid arthritis, Psoriasis, Ankylosing Spondylitis, Sarcoidosis, Idiopathic Pulmonary Fibrosis, Chronic Fatigue Syndrome and other complex disorders with a chronic autoimmune inflammatory component. The vaccine, peptides, polypeptides, polynucleotides and vectors disclosed herein may therefore be used in the prevention or treatment of any of these specific conditions.

The subject being treated may in some instances be identified by a clinical diagnostic for MAP infection. In one embodiment, the MAP infection is detected by a clinically-applicable simple diagnostic for human and animal MAP infection using monoclonal antibodies as described in International Patent Application No. PCT/GB2018/050075, which is incorporated herein by reference in its entirety. For example, one or two pairs of mutually-exclusive monoclonal antibodies that target specific peptide sequences in the accessible amino- and carboxy-terminal extracellular domains of the multi-copy IS900-encoded protein may be used to detect MAP in a sample from a subject. In this diagnostic test, one antibody of a pair recognises the native peptide sequence and the other its phosphorylated derivative. Simultaneous use of two or more differentially fluorophore-labelled monoclonals results for the first time in bright clear images of MAP packing the cytoplasm of MAP infected host cells and displayed on infected cell surfaces. The diagnostic method may, for example, use a pair of monoclonal antibodies directed to MVINDDAQRLLSQR and MVINDDAQRLL[pS]QR and/or a pair of monoclonal antibodies directed to YLSALVSIRTDPSSR and YLSALVSIRTDPS[pS]R. The antibodies may for example be labelled with the same or different fluorophores.

Flow Cytometry of blood samples using of these pairs of specific co-localising monoclonal antibodies provides a simple accurate detection method that allows quantification of MAP-infected circulating white blood cells and their subtypes. Use of these fluorophore labelled antibodies on tissue samples such as endoscopic biopsies and surgically resected tissues reveals strong images of brightly lit MAP infected cells and their distribution and host cell subtypes.

These specific reagents and resulting new clinical MAP tests on blood and tissues have shown that everyone with Crohn's disease is infected with MAP. The methods and reagents provide for the first time simple accurate tests for the diagnosis and quantification of MAP infection in humans and animals. They can also be applied to clinical screening blood and tissue samples from people with other diseases where MAP is a candidate pathogen like Parkinson's disease, Alzheimer's disease, Multiple Sclerosis, Irritable Bowel Syndrome, Ulcerative colitis, type 1 Diabetes Mellitus, Thyroiditis, Rheumatoid arthritis, Psoriasis, Ankylosing Spondylitis, Sarcoidosis, Idiopathic Pulmonary Fibrosis, Chronic Fatigue Syndrome and other complex disorders with a chronic autoimmune inflammatory component (Hui K Y et al. 2018; Sci. Transl. Med. 10, eaai7795; Hütlova A et al. 2018. The EMBO Journal e98694).

Accordingly, the present invention relates to a vaccine, peptide, polypeptide, polynucleotide, vector, cell or composition as disclosed herein for use in a method of therapy, in particular in a method or treating or preventing a disease, disorder or symptoms associated with or caused by a MAP infection. These molecules of the invention may thus also be used in the manufacture of a medicament for treating or preventing such a disease, disorder or condition. Thus, the present invention also encompasses the use of a vaccine, peptide, polypeptide, polynucleotide or vaccine vector according to the invention in the manufacture of a medicament for treating or preventing MAP infection or a condition or symptom associated with MAP infection. In particular, the molecules of the invention are proposed for the treatment or prevention of a chronic inflammation of the intestine, preferably in a mammal such as a human, cow, sheep or goat. The invention thus also provides a method of treating or preventing any such disease, disorder or symptom comprising administering to a subject in need thereof a polypeptide, polynucleotide, expression cassette, vector, cell, antibody or composition of the invention.

The present invention is broadly applicable to vaccination methods and is relevant to the development of prophylactic and/or therapeutic vaccines (including immunotherapeutic vaccines). It is to be appreciated that all references herein to treatment include curative, palliative and prophylactic treatment.

According to the present invention, the peptide, polynucleotide, vector, or vaccine may be employed alone as part of a composition, such as but not limited to a pharmaceutical composition or a vaccine composition or an immunotherapeutic composition to prevent and/or treat a condition associated with MAP infection. The administration of the composition may be for either “prophylactic” or “therapeutic” purpose. As used herein, the term “therapeutic” or “treatment” includes any of following: the prevention of infection or reinfection; the reduction or elimination of symptoms; and the reduction or complete elimination of a pathogen. Treatment may be effected prophylactically (prior to infection) or therapeutically (following infection).

Prophylaxis or therapy includes but is not limited to eliciting an effective immune response to a polypeptide of the invention and/or alleviating, reducing, curing or at least partially arresting symptoms and/or complications resulting from or associated with a MAP infection. When provided prophylactically, the composition of the present invention is typically provided in advance of any symptom. The prophylactic administration of the composition of the present invention is to prevent or ameliorate any subsequent infection or disease. When provided therapeutically, the composition of the present invention is typically provided at or shortly after the onset of a symptom of infection or disease. Thus the composition of the present invention may be provided either prior to the anticipated exposure to MAP or onset of the associated disease state or after the initiation of an infection or disease.

The vaccine is typically considered to be prophylactically effective if it reduces the MAP load in a vaccinated subject compared to an un-vaccinated subject after post-vaccination challenge with MAP. Where the subject is infected with MAP prior to vaccination, the vaccine is typically considered to be effective if MAP load is reduced compared to before vaccination. MAP load in blood or tissues may be determined. The MAP and/or MAP components, e.g. peptides or proteins, may be present on the surface of cells or intracellularly within the cytoplasm. MAP expression may be determined by antibody staining (WO 2018/130836).

The effectiveness of the vaccine may be determined in any suitable way. For example, changes in cytokine expression, T-cell activation, antibody production and/or peripheral blood mononuclear cell (PBMC) MAP-killing may be used to determine the effectiveness of the vaccine. Where cytokine expression is monitored, for example, a decrease in inhibitory cytokine production, such as IL-10 production, is a desirable effect. The generation of T-cells specific for epitopes in the vaccine may, for example, be detected by stimulating T-cells ex vivo with a peptide present in the vaccine and detecting IFN-γ release, for example using an ELISpot or ELISA assay. Suitable assays for detecting antibodies specific to the polypeptide in the vaccine, for example, antibodies to one or more peptides present in the vaccine, are known in the art. Similarly, ex vivo assays for monitoring PBMC MAP-killing are known.

### Subject to be Treated

The present invention relates in particular to the treatment or prevention of diseases or other conditions which are associated with infection by MAP. These treatments may be used on any animal which is susceptible to infection by MAP.

The subject to be treated may be any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly. If a mammal, the subject will preferably be a human, but may also be a domestic livestock, laboratory subject or pet animal.

The subject to be treated may thus be any vertebrate that is susceptible to infection by MAP. Numerous animals have been shown in the art to be capable of such infection, including livestock such as cattle, goat and sheep, primates such as macaques and humans, other mammals including alpaca, antelope, ass, elk, horses, deer, dogs, gerbils and rabbits, and birds including the chicken. The compositions of the present invention may thus be used in the treatment of any such species.

### Combined Therapy

In one embodiment, the method of treating or preventing MAP infection or a condition associated with MAP infection may comprise administering a further therapeutic agent which has activity against MAP or a further therapeutic agent used in the treatment of a condition which is associated with MAP infection to the subject.

The further therapeutic agent may be another polynucleotide, vector or polypeptide, for example, one or more antimicrobial agent, such as a combination including Rifabutin and Clarithromycin, is administered to the patient, either alone or in combination with one or more additional therapeutic agents. The treatment may be a prophylactic or therapeutic MAP vaccine. The treatment may comprise passive immunotherapy administering to the subject anti-MAP monoclonal antibodies such as the antibodies and peptides described herein.

The therapeutic agent may be, for example an agent which has activity against MAP, or an agent used in the treatment of a condition which is associated with MAP infection. The vaccine, peptide, polypeptide, polynucleotide or vaccine vector of the invention is preferably administered in an amount which is sufficient to augment the anti-MAP effects of the other therapeutic agent or vice versa. Numerous other agents may be used in the treatment of MAP or conditions which are associated with MAP infection. These include the rifamycins such as rifabutin and rifaximin, clarithromycin and other macrolides, azathioprine, methotrexate, Humira, 6-mercaptopurine and/or Infliximab. Various anti-tuberculosis drugs may also be used.

The other therapeutic agent may be an agent which potentiates the effects of the molecule of the vaccine, peptide, polypeptide, polynucleotide or vaccine vector the invention. For example, the other agent may be an immunomodulatory molecule or an adjuvant which enhances the immune response to the polypeptide. Alternatively, the other molecule may increase the susceptibility of MAP present in the subject to attack, such as attack from the immune system.

In one embodiment, therefore, the vaccine, peptide, polypeptide, polynucleotide or vaccine vector of the invention is used for therapy in combination with one or more other therapeutic agents.

The vaccine, peptide, polypeptide, polynucleotide or vaccine vector may be administered separately, simultaneously or sequentially. The vaccine, peptide, polypeptide, polynucleotide or vaccine vector may be administered in the same or different compositions. Accordingly, in a method of the invention, the subject may also be treated with a further therapeutic agent.

A composition may therefore be formulated which comprises the vaccine, peptide, polypeptide, polynucleotide or vaccine vector of the invention and also one or more other therapeutic molecules. For example, a vector of the invention may be formulated with another vector which encodes one or more other antigens or therapeutic molecules. The vaccine, peptide, polypeptide, polynucleotide or vaccine vector of the invention may alternatively be formulated with one or more therapeutic proteins.

The present invention also encompasses the use of a vaccine, peptide, polypeptide, polynucleotide or vaccine vector according to the invention in combination with an additional therapeutic agent in the manufacture of a medicament for treating or preventing MAP infection or a condition or symptom associated with MAP infection.

### Delivery Methods

Once formulated the vaccine compositions can be delivered to a subject in vivo using a variety of known routes and techniques. For example, a composition can be provided as an injectable solution, suspension or emulsion and administered via parenteral, subcutaneous, epidermal, intradermal, intramuscular, intraarterial, intraperitoneal, intravenous injection using a conventional needle and syringe, or using a liquid jet injection system. Compositions can also be administered topically to skin or mucosal tissue, such as nasally, intratracheally, intestinal, rectally or vaginally, or provided as a finely divided spray suitable for respiratory or pulmonary administration. Other modes of administration include oral administration, suppositories, and active or passive transdermal delivery techniques. Particularly in relation to the present invention, compositions may be administered directly to the gastrointestinal tract.

Alternatively, the compositions can be administered ex vivo, for example delivery and reimplantation of transformed cells into a subject are known (e.g., dextran-mediated transfection, calcium phosphate precipitation, electroporation, and direct microinjection into nuclei).

### Delivery Regimes

The compositions are administered to a subject in an amount that is compatible with the dosage formulation and that will be prophylactically and/or therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed.

As used herein, the term “prophylactically or therapeutically effective dose” means a dose in an amount sufficient to elicit an immune response to one or more epitopes of a polypeptide of the invention and/or to alleviate, reduce, cure or at least partially arrest symptoms and/or complications from a disease, such as an inflammatory bowel disorder, which is associated with a MAP infection.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one embodiment, a single dose is administered on a single occasion. In an alternative embodiment, a number of doses are administered to a subject on the same occasion but, for example, at different sites. In a further embodiment, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

In one embodiment, different compositions of the invention may be administered at different sites or on different occasions as part of the same treatment regime. It is known that improved immune responses may be generated to an antigen by varying the vectors used to deliver the antigen. There is evidence that in some instances antibody and/or cellular immune responses may be improved by using two different vectors administered sequentially as a “prime” and a “boost”.

For example, the same peptide, polypeptide, or polynucleotide of the invention may be administered as a “prime” in one composition, and then subsequently administered as a “boost” in a different composition. The two vaccine compositions used for the “prime” and “boost” may differ. For example, they may differ in the choice of vector comprising the polynucleotide. For example, two or more of different vectors each selected from plasmid vectors, poxvirus vectors, adenovirus vectors or other vectors as described herein may be administered sequentially.

In one embodiment, a “prime” is effected by administering a polynucleotide of the invention in a plasmid vector such as pSG2. A “boost” is then effected at a later time using a polynucleotide of the invention in a poxvirus vector such as MVA.

In an alternative embodiment a “prime” is effected by administering a polynucleotide of the invention in an adenovirus vector such as Ad5. A “boost” is then effected at a later time using a polynucleotide of the invention in a poxvirus vector such as MVA.

The vaccine composition used for the “prime” may differ from the vaccine composition used for the “boost” in that one composition may comprise a peptide or polypeptide and the other composition may comprise a polynucleotide. For example, in one embodiment, the “prime” may be effected by administering a peptide or polypeptide and the “boost” may be effected by using a polynucleotide. As a specific example of this, the “prime” may comprise administering a peptide or polypeptide, for example a peptide comprising, consisting essentially of or consisting of the sequence shown in any one of SEQ ID NOs: 3 to 6, and the “boost” may be effected by administering a polynucleotide comprising an N-terminal fragment of P900, such as a polynucleotide comprising an N-terminal fragment of P900 and one or more of an ahpC polypeptide, a gsd polypeptide, a p12 polypeptide and a MPA polypeptide. The polypeptide may have one of the specific sequences shown in SEQ ID NO: 41 and SEQ ID NO: 42, in which the MAP P900 N-terminal fragment is inserted at the junction between the aphC, and gsd polypeptides, between the gsd and p12 polypeptides, between the p12 and mpa polypeptides, before the aphC polypeptide and/or after the mpa polypeptide. It is envisaged that such an immunisation protocol may be particularly beneficial in immunising livestock, such as cattle or sheep, particularly where a phosphorylated form of the MAP P900 N-terminal fragment is used, such as a peptide comprising, consisting of, or consisting essentially of the amino acid sequence shown in SEQ ID NO: 4 or 6.

In such a prime-boost protocol, one or more administrations of the prime and/or the boost may be performed. For example, the prime and/or boost step may be achieved using a single administration or using two or more administrations at different sites and/or on different occasions. In one embodiment, two administrations on different occasions are given for the prime step and a single administration on a later occasion is given for the boost step.

Different administrations may be performed on the same occasion, on the same day, one, two, three, four, five or six days apart, one, two, three, four or more weeks apart. Preferably, administrations are 1 to 5 weeks apart, more preferably 2 to 4 weeks apart, such as 2 weeks, 3 weeks or 4 weeks apart. The schedule and timing of such multiple administrations can be optimised for a particular composition or compositions by one of skill in the art by routine trials.

Dosages for administration will depend upon a number of factors including the nature of the composition, the route of administration and the schedule and timing of the administration regime. Suitable doses of a molecule of the invention may be in the order of up to 15 μg, up to 20 μg, up to 25 μg, up to 30 μg, up to 50 μg, up to 100 μg, up to 500 μg or more per administration. For some molecules of the invention, such as plasmids, the dose used may be higher, for example, up to 1 mg, up to 2 mg, up to 3 mg, up to 4 mg, up to 5 mg or higher. Such doses may be provided in a liquid formulation, at a concentration suitable to allow an appropriate volume for administration by the selected route. In the case of a viral vector, a dose of about 10⁶, 10⁷, 10⁸, 10⁹, 10¹⁰ or more pfu may be given per administration. For example, a dose of 10⁹ pfu or 25 μg of a vector of the invention may be administered in a 50 μl dose at multiple sites and/or on multiple occasions.

### Kits

The invention also relates to a combination of components described herein suitable for use in a treatment of the invention which are packaged in the form of a kit in a container. Such kits may comprise a series of components to allow for a treatment of the invention. For example, a kit may comprise two or more different vectors of the invention, or one or more vectors of the invention and one or more additional therapeutic agents suitable for simultaneous administration, or for sequential or separate administration such as using a prime and boost protocol. The kit may optionally contain other suitable reagent(s), control(s) or instructions and the like.

### EXAMPLES

### Example 1: Efficacy of hAd5 HAV Prime and MVA HAV Boost Vaccination in Bovine Calves

A BBSRC-funded trial of HAV vaccination in protection against experimental MAP infection was carried out (2010-2014) by St George's University of London, The Jenner Institute University of Oxford, The Roslin Institute University of Edinburgh, Animal Health and Welfare and the Agri-Food and Biosciences Institute of Northern Ireland.

The amino acid sequence of the HAV vaccine insert with leader peptide and PK tail at either end in bold is shown below. The sequences from its four MAP genes 1589c (AhpC), 1234 (Gsd), 2444c (p12) and 1235 (mpa) which have been described above, comprise the peptide sequences in between. The junctions between the HAV vaccine components are marked *.

MQIFVKL*PLLTIGDQFPAYELTALIAGDLSKVDAKQPGDYFTTVTSEDH
AGKWRVVFFWPKDFTGPEIATFGKLNDEFEDRDAQVLGVSIDSEFVHFNW
RAQHEDLKNLPFPMLSDIKRELSLATGVLNADGVADRATFIVDPNNEIQF
VSVTAGSVGRNVEEVLRVLDALQSDELCACNWRKGDPTLNATELLKASAL
*GSIVGQTYREVEVVLVDGGSTDRTLDIANSFRPELGSRLVVHSGPDDGP
YDAMNRGVGVATGEWVLFLGADDTLYEPTTLAQVAAFLGDHAASHLVYGD
VVMRSTKSRHAGPFDLDRLLFETNLCHQSIFYRRELFDGIGPYNLRYRVW
ADWDFNIRCFSNPALITRYMDVVISEYNDMTGFSMRQGTDKEFRKRLPMY
FWVAGWETCRRMLAFLKDKENRRLALRTRLIRVKAVSKERSAEP*RIRRH
RHAEIILSMPGFGVILGAEFLAATGGDMAAFASADRLAGVAGLAPVPRDS
GRISGNLKRPRRYDRRLLRACYLSALVSIRTDPSSRTYYDRKRTEGKRHT
QAVLALARRRLNVLWAMLRDHAVYHPATTTAAARL*KLRRGERPMSLGQV
FDPRANALHSFPLTGRMPWAPFIVSSWLRNPHPAQYFTARCLRILPGLWI
GAQGGSAAKLLMSGAPIEYVLKDSAVWMFKFDIGGTPRDIPVAGIWNGSL
WTPAWGGIHAIASNAYQFRNVIPARWSVSSAVLPNYRLVAALPMAYHNQR
MRFRTDLSYGVYGFAEINPIALVEKPALSWKSRLRRKNSSIALANMEDGG
SVGRSNDIPGRRARFIGEKAEDPPAPSPR*PALRIPNPLLGLD

The underlined sequence is that of p12, the carboxyterminal region of the P900 protein encoded by the positive strand of the IS900 element. The p12 portion within HAV has a short cytoplasmic domain followed by the highlighted predicted transmembrane-like sequence, followed by the remaining extracellular portion of the P900 protein. The extracellular carboxyterminal portion of P900 is exported by the MAP cell and further cleaved by limited proteolysis to traffic between host cells in exosome like vesicles. The IS900 derived component of the HAV vaccine has the highest level of cellular immunogenicity of the 4 HAV vaccine components.

Six Holstein Friesian calves were given the priming dose of 10⁹ viral particles (vp) of hAd5 HAV (the AhpC, gsd, p12 and mpa antigen expressing adenovirus vaccine described in WO 2007/017635) in 1 mL sterile PBS by intradermal injection (id) into the skin of the neck. Five control calves were given the same dose of hAd5 expressing Green Fluorescent Protein (GFP). At 6 weeks calves in the test group received the boosting dose of 10⁹ plaque forming units (pfu) of MVA HAV in 1 mL sterile PBS id. Control calves received the same id dose of MVA GFP.

At 12 weeks all animals received 5×10⁸ live virulent MAP strain R0808 given orally in 20 ml PBS on 2 consecutive days. They were then followed for 38 weeks. No adverse effects of vaccination or inflammatory disease over the period of the study were seen in any of the animals. Blood and faecal samples were obtained before and after each prime and boost vaccination and MAP challenge. Observations were continued with monthly blood and faecal sampling for a period of 38 weeks at the end of which the animals were euthanized.

Shortly after oral MAP challenge all animals in the control group shed MAP in their faeces and continued throughout the study. In all test animals HAV vaccination prevented detectable faecal shedding of MAP throughout the study. All six calves in the test group responded with an increased PBMC release of IFN-γ following PPD-J stimulation not seen in the control group. This was accompanied by a rise in the percentage of CD4+IFN-γ+ and CD8+IFN-γ+ secreting cells which was absent from the control group. Specific cellular immune responses to HAV vaccine peptides were seen in all HAV vaccinated but not in control animals two weeks after boosting and were maintained throughout the study.

Immediately prior to the oral administration of MAP, laboratory tests of the ability of PBMC from control calves to kill MAP were the same as those of PBMC from HAV vaccinated calves. Within 1 week of challenge the in vitro MAP killing capacity of PBMC from control calves dropped dramatically by 30% but was unchanged in HAV vaccinated calves. At the same time circulating PBMC from 5/6 HAV vaccinated and 4/5 control calves tested PCR positive for MAP.

A significant impairment of the in vitro capacity of PBMC from control calves to kill MAP remained and by week 14 the proportion of these animals with MAP positive PBMC in their blood remained at 4/5. At the same time MAP positive PBMC in blood of vaccinated calves had fallen to 2/6. These trends in blood continued so that over the 19 week second half of the study following MAP infection 3/5 control animals became consistently MAP positive and the other 2 intermittently positive. By contrast PBMC from 4/6 HAV vaccinated calves remained consistently MAP negative. Each of the other 2 animals had only 1 of 5 MAP PCR tests positive over the last 19 week period of observation. ELISpot responses of PBMCs to stimulation with HAV specific peptide antigens which were absent from the control group of animals, continued throughout the study in all vaccinated animals.

At autopsy, full thickness tissue samples were taken from 11 sites throughout the length of the small intestine comprising 2 from the duodenum, 6 from the jejunum and 3 from the ileum together with 4 mesenteric lymph nodes (MLN) and tissue from the spleen. MAP loads in tissues at autopsy were measured by specific qPCR. In the 5 control calves all tissue samples were positive for MAP with microbial loads of up to 5 logs per gram of sample. As a group these tissue samples comprised totals of 10 from the duodenum, 30 from the jejunum, 15 from the ileum, 20 from mesenteric lymph nodes (MLN) and 1 sample each for the 5 spleens.

By contrast, in the 6 HAV vaccinated calves 10 of 12 tissue samples from the duodenum, 28 of 36 from the jejunum, 12 of 18 from the ileum, 7 of 24 from MLN and 3 of the 6 spleens tested negative for MAP. In the residual MAP positive samples, HAV vaccination was associated microbial loads substantially lower than corresponding samples in the control group. As with other chronic enteric human pathogens such as Tuberculosis, Yersinia, Legionella and others, MAP demonstrates an ability to persist in MLN for which further strategies may be devised.

This study was necessarily designed to test the ability of HAV vaccination in a protective role against MAP infection in bovine calves. Both the control and HAV Vaccinated group of calves became MAP positive in blood after MAP challenge. All control calves remained MAP positive in blood and shed MAP in their faeces throughout the study. All HAV vaccinated calves appeared to eliminate MAP from blood and blocked detectible faecal shedding.

### Example 2: Phases of Development of MAP Antibodies

### Phase 1. Mapping Mouse Antibody Binding Peptide Domains in P900

IS900 (NCBI accession: AE016958.1) is a DNA insertion element of 1451-53 bp discovered by the present inventor and colleagues late in 1985 in three Crohn's disease isolates of MAP (E. Green et al Nucleic Acids Research 1989; 17: 9063-73). It is present in MAP in 14-18 identical copies inserted at highly conserved sites throughout the MAP genome. This multicopy element has its own promoter, is abundantly expressed in humans and contributes to the broad pathogenic phenotype.

The positive strand of IS900 predicts a protein (P900) of 406 amino acids. Its full length amino acid sequence is unique to MAP but there are P900 ‘look-alikes’ in closely related mycobacteria and actinomycetes which cover most of the P900 molecule.

Full length P900 protein encoded by the positive strand of IS900 is toxic for E. coli cells. A less toxic truncated version consisting of amino acids 49 to 377 of P900 was made and expressed as the recombinant protein in E. coli.

Ten mice were immunised with the recombinant truncated P900 protein adherent to magnetic beads because the free recombinant protein was found not induce a satisfactory immune response. The sera from immunised mice were screened by ELISA against immobilised P900 and 4 positive mice were identified. Spleen cells from these mice were used for hybridoma fusion resulting in 10 parental clones. Supernatants from these and their successive subclones were screened against a library of 64 synthetic 15 amino acid peptides overlapping by 10 amino acids spanning the truncated P900 amino acid sequence from ELIAAVTTLADGGEV . . . to . . . DRKRTEGKRHTQAVL. The antibodies were all IgM and the clones eventually proved unstable. Despite the inability to obtain the desired monoclonal reagents, 8 peptides or peptide clusters were identified as immunogenic within the truncated P900 protein. In the peptide library these involved peptides No. 2-VTTLADGGEVTWAID, 27-NKSRAALILLTGYQT, 41-AKEVMALDTEIGDTD, 42-ALDTEIGDTDAMIEE and a cluster within the sequence

GRISGNLKRPRRYDRRLLRACYLSALVSIRTDPSSRTYYD.

### Phase 2. Preparation of Polyclonal Antibodies to P900 Sequences in Rabbits and Their Testing on Humans and Animals

### Polyclonal Antibody Preparation

Initial peptides designated A1-VTTLADGGEVTWAIDLNA, A2-NKSRAALILLTGYQTPDA, A3-NLKRPRRYDRRLLRAGYL, and A4-YLSALVSIRTDPSSR were identified. These were prepared as synthetic branched octapeptide immunogens on polylysine cores and used to immunise rabbits. Suitable titres of polyclonal antibodies were readily achieved for A1, A3, and A4. A2 was not immunogenic the rabbit. A2 was also intracellular in MAP and was not studied further.

Antibodies in A1 and A3 sera reacting with Freund's complete adjuvant (M tuberculosis H37Ra, Difco, USA) were abstracted to completion using excess antigen. Only Freund's incomplete antigen was used as the adjuvant with A4. Rabbit polyclonal reagents A1, A3 and A4 were applied in preliminary studies to explore their ability to detect their target sequences and therefore MAP immunoreactivity in human and animal tissues and human blood.

### MAP Immunoreactivity in Human Tissues

In an initial study, fresh intestinal mucosal biopsies were obtained from 14 patients diagnosed with Crohn's disease (CD) attending the endoscopy clinic at St Thomas' Hospital, London, UK and 10 control patients without inflammatory bowel disease (nIBD) attending for screening or follow up. Ethical approval was given by the Local Ethics Committee (EC03/053). Biopsies were embedded in Jung tissue freezing medium and snap frozen in liquid nitrogen in the endoscopy suite. They were then taken to the laboratory where they were coded and stored −80° C. prior to use.

Orientated biopsies were subsequently cut in 6 μm sections and mounted on PTFE coated slides and stained with A1, A3 and A4 polyclonal antibodies at a dilution of 1:400 to 1:800. Host cell phenotypic markers were CD3 for T cells, CD8 for monocytes/macrophages, CD19 for B cells, CD66b for neutrophils, CD83 for Dendritic cells, PgP9.5 for Glial cells and CD31 for endothelium. Secondary antibodies were rabbit anti-mouse TRITC (R0270 Dako, UK), rabbit anti-mouse FITC (F0261, Dako), swine anti-rabbit TRITC (R0156, Dako), swine anti-rabbit FITC (F0205, Dako) and goat anti-mouse FITC (F0479). Slides were washed ×3 in PBS and mounted in Fluoromount agent (F4680 Sigma-Aldrich, UK) followed by a coverslip.

Use of antibodies A1, A3 and A4 alone at concentrations of 1:500 to 1:800 resulted in staining of cells within the epithelium and in the underlying lamina propria. Antibodies were then used in pairs with A1 labelled with TRITC (red) and A3 or A4 with FITC (green).

The A1 site is located on the extracellular aminoterminal domain of the P900 protein adjacent to the first transmembrane region and right up against the surface of the microbial cell. The A1 peptide appeared to remain attached to MAP.

The A3 and A4 sites are located at the centre of the longer carboxyterminal extracellular domain either side of Cysteine 344. The carboxyterminal domain may either be attached or released by limited proteolytic cleavage close to the second transmembrane region.

Use of A1 (red) and A4 (green) with the carboxyterminal peptide still attached resulted in colocalisation (gold) not only in the same cells but also on submicrometre particles within the cytoplasm of infected cells. Release of the carboxyterminus resulted in a progressive gradient of colour change from gold to orange to red and the visible migration of the released peptide (green) in the cytoplasm of the affected cell. Other cells were seen to contain green only suggesting the ability of released carboxyterminal peptide to traffic to other cells which did not themselves contain MAP. This was supported by the appearance in tissues of intercellular vesicles filled green consistent with endosomes.

In the surface epithelium MAP was seen to infect enterocytes as well as intra-epithelial cells consistent with lymphocytes and macrophages. MAP was seen to cluster often in a ‘necklace’ around the base of the mucus vacuole of goblet cells releasing green carboxyterminal peptides which migrated in the cytoplasm to the apex of these cells as well as within the mucus vacuole itself.

MAP was also seen to infect cells widely in the lamina propria and particularly clusters of cells around the bases of crypts. Staining involved particularly macrophages, polymorphs and B-lymphocytes but not T-lymphocytes although the presence of T-lymphocytes adjacent to MAP clusters was frequently noted.

Abundant MAP infection in endoscopic mucosal biopsies was seen in all 14 patients with Crohn's disease. Scant clusters of immunoreactive MAP were seen in 8 out of the 10 control subjects. The other 2 control subjects contained no MAP staining at all. The addition of specific peptide to the operational buffer completely blocked staining of tissues by the corresponding antibody. Use of other peptides had no effect on antibody binding. Together with colocalisation this specific blockade reinforced the precision and specificity of the MAP detection system.

### PCR Verification of A1 Antibody Binding to MAP in Human Tissues

Lasermicrodissection pressure catapulting (LMPC) of A1 immunoreactive loci was carried out to determine whether antibody recognition in tissues equated with the presence of MAP using IS900 PCR. Fresh endoscopic mucosal biopsies were obtained from 11 consenting patients and 4 control subjects without inflammatory bowel disease. Tissues were snap frozen in liquid nitrogen and 6 μm cryostat sections were cut as previously described. Sections were transferred to PTFE-coated microscope slides for routine H&E staining. Those for LMPC were immobilised on PEN-membrane slides (Carl Zeiss MicroImaging GmbH, Germany).

Immunoreactive MAP regions were identified in sections with A1 rabbit polyclonal antibody using biotinylated alkaline phosphatase H tagged 2^{nd} antibody to rabbit immunoglobulin. After washing slides in PBS, Vectastain Universal ABC-AP kit (Vector Laboratories UK) was used for localisation of immunoreactive MAP regions according to the manufacturer's instructions. Secondary antibodies were localised using the Vector Blue Alkaline Phosphatase Substrate Kit 1 (Vector Laboratories UK).

Lasermicrodissection and pressure catapulting was used to isolate immunoreactive (IR) and non-immunoreactive (nIR) MAP regions using the Zeiss PALM MicroBeam Laser microdissection system. Prior to microdissection, particular care was taken to ensure that sections were completely air dried so that excised regions readily detached. IR and nIR regions were identified visually and the adhesive cap tube positioned above the selected area. Samples were accumulated onto the cap of the adhesive tube. DNA extraction was carried out as described (T. Bull et al. 2003 J Clin Microbiol 2003; 41:2915-23). Briefly, 200 μL of Mycobacterium Lysis Buffer (MLB), 8.6 ml molecular-grade water, 800 μL 5M NaCl, 1M 10×Tris-EDTA (TE) and 600 μL 10% SDS was added to each tube and incubated overnight at 37 deg C. 10 μL of 10 mg/ml Proteinase K (Sigma), 5 μL of 100 mg/ml Lysozyme (Sigma) and 4 μL of 120 mg/ml Lipase (Sigma) in MLB were added to each tube and incubated at 37° C. for a further 3 hours. Samples were transferred to Lysing Matrix B ribolyser tubes and 400 μL 1×TE added. Tubes were mechanically disrupted at 6.5 ms² for 45 seconds on a FastPrep Ribolyser instrument. Standard phenol-chloroform-isoamyl DNA extraction procedure was carried out. Purified DNA was resuspended in 50 μL 1×TE. Nested PCR using 2 μL of template DNA was carried out using L1 and L2 first round primers and AV1 and AV2 second round primers. The expected 298 bp PCR amplicon was visualised using 1% agarose gel electrophoresis. Stringent precautions were taken as described to exclude amplicon contamination.

All 4 control subjects tested negative by IS900 PCR. Immunoreactive regions from 7 of the 11 patients in the CD group were positive for MAP by IS900 PCR, confirmed by amplicon sequencing in 5. All the nIR regions sampled in CD patients were PCR negative. All 7 patients testing positive for MAP by IS900 PCR were undergoing treatment with azathioprine alone. The 4 PCR negative samples came from Crohn's disease patients receiving treatment with azathioprine in combination with Humira or 6-mercaptopurine and Infliximab.

### Phase 3. Preparation of Murine Monoclonal Antibodies to Optimised P900 Peptide Sequences Within the Selected A0, A1, A3 and A4 Sites and Phosphorylated Derivatives

At this stage, an additional target site for monoclonal antibody production designated A0 was introduced comprising the sequence MVINDDAQRL, residues 26-39 in the extracellular aminoterminal domain of P900. Few identical matches to this sequence were found in NCBI databases.

The production of murine monoclonal antibodies was first attempted in the following manner. Immunogen peptides in each case incorporating a solitary Cys thiol for linkage to KLH were prepared for A0 (MVINDDAQRL-C), A3 (C-NLKRPRRYDR) and A4 (C-VSIRTDPSSR) and 5 mice were immunized in each group. Despite good immunological responses in some of the mice in each group, no monoclonals recognising their native targets in tissues were obtained. This was found to be due to the exclusive use of the target peptide for screening ELISAs and clonal selection being coated directly on to ELISA plate wells. This resulted in substantial artifact and the project was a comprehensive failure.

On the other hand the inventor found that it was essential for the target synthetic peptides used in screening ELISAs to be alpha-n Biotinylated and attached to wells coated with streptavidin. This increased the steric accessibility of the attached mobile peptides and permitted the adoption of the appropriate configuration of the peptide for antibody in the liquid phase. There was close correlation between antibody binding to target peptide in this form in ELISAs and to the native peptide in target tissues.

The essential Streptavidin coating and Biotinyl-peptide immobilisation in ELISA wells was adopted and used throughout the next project. During this project, mouse sera and culture supernatants were selected for binding to Reference peptide but not Negative peptides. Selected samples were subsequently tested by immunofluorescence on human and animal tissues and cells infected with MAP.

Immunogens were synthesised using the peptide sequences A0X MVINDDAQRLLSQR-C, A1 VTTLADGGEVTWAID-C, and XA4 YLSALVSIRTDPSSR-C in each case the Cys thiol was used to link to KLH using standard methods. These constructs were used to immunise groups of 5 to 10 BalbC mice. Good serological responses to immunisation occurred in all groups and promising candidate clones were obtained for each group. Despite additions to immunisation protocols including in vitro immunisation and follow on immunisation using different immunogen adducts together with much additional work, no suitable final stable IgG clones could be obtained.

The materials used initially in the next project were as follows:

| | |
|---|---|
| Immunogen Peptide | A0 ac-MVINDDAQRL-8branchedPolylysineOctamer |
| Reference peptide | Biotinyl-MVINDDAQRL-amide |
| Negative peptide 1 | Biotinyl-MVINDDLQR-amide |
| Negative peptide 2 | Biotinyl-MVINNDAE-amide |
| Immunogen peptide | A1 ac-VTTLADGGEVT-8branchedPolylysineOctamer |
| Reference peptide | Biotinyl-VTTLADGGEVT-amide |
| Negative peptide 1 | Biotinyl-VATMADGGEVT-amide |
| Negative peptide 2 | Biotiny1-VTRLADGGEVT-amide |
| Immunogen peptide | A3 ac-NLKRPRRYDR-8branchedPolylysineOctamer |
| Reference peptide | Biotinyl-NLKRPRRYDR-amide |
| Negative peptide 1 | Biotinyl-NLKRPRR-amide |
| Negative peptide 2 | Biotinyl-NLRRPRRYHR-amide |
| Negative peptide 3 | Biotiny1-NLHRPRRYHR-amide |
| Negative peptide 4 | Biotiny1-NMRRPRRYNR-amide |
| Negative peptide 5 | Biotinyl-NLRRPKRYNR-amide |
| Negative peptide 6 | Biotinyl-NLQRPRRYNR-amide |
| Immunogen peptide | A4 ac-VSIRTDPSSR-8branchedPolylysineOctamer |
| Reference peptide | Biotinyl-VSIRTDPSSR-amide |
| Negative peptide 1 | Biotiny1-VSIRTDP-amide |
| Negative peptide 2 | Biotiny1-SIRSDPSSR-amide |
| Negative peptide 3 | Biotinyl-YSIRSDPASR-amide |
| Negative peptide 4 | Biotinyl-VSVRYDPSSR-amide |
| Negative peptide 5 | Biotiny1-IAIRTDPASR-amide |

Groups of 5 Swiss Webster mice were immunised with the immunogen peptide constructs in Freund's Complete Antigen on day 1 followed by 2 booster shots on day 14 and day 21 using Freund's Incomplete antigen. Boosting was continued but it was clear that none of the 4 groups were responding satisfactorily. Fresh peptide immunogens ac-MVINDDAQRL-C, ac-VTTLADGGEVT-C, ac-NLKRPRRYDR-C, and ac-VSIRTDPSSR-C were synthesised, coupled via the C- to KLH and immunisations continued.

Transient responses in the A0 and A1 groups were not sustained. Both flatlined and were terminated.

Serum from one mouse in each of the A3 and A4 groups achieved a sufficient titre to proceed to fusion and development of parental clones. A satisfactory subclone was not subsequently obtained for A3 and this project was terminated. A satisfactory subclone was achieved for A4 which recognised the reference peptide and none of the 5 negative peptides and was taken through final production and Protein A affinity purification.

At this stage three further modifications were introduced into the protocol:
1. The use of Balb/C mice.
2. Adoption of the technique of administering the immunogen at the base of the tail vein followed by direct fusion of pooled cells from inguinal lymph nodes.
3. Redesigned projects with the following new peptide immunogens.

| | |
|---|---|
| Immunogen peptide | A0X C-MVINDDAQRLLSQR-amide |
| Reference peptide | Biotinyl-MVINDDAQRLLSQR-amide |
| Negative peptide 1 | Biotiny1-MVINDDLQRIILFL-amide |
| Negative peptide 2 | Biotinyl-MSINDDAQKLKDRL-amide |
| Immunogen peptide | AOXP C-MVINDDAQRLL[pS]QR-amide BSA conjugated |
| Reference peptide | Biotiny1-MVINDDAQRLL[PS]QR-amide |
| Negative peptide 1 | Biotiny1-MVINDDAQRLLSQR-amide |
| Immunogen peptide | XA1 ac-AAVTTLADGGEVTWAIDGKK-C BSA conjugated |
| Reference peptide | Biotinyl-KKGAAVTTLADGGEVTWAID-amide |
| Negative peptide 1 | Biotinyl-KKGAAGTTLADGGEVTWAID-amide |
| Negative peptide 2 | Biotinyl-KKGSTVATMADGGEVTWAID-amide |
| Negative peptide 3 | Biotinyl-KKGQAVTRLADGGEVTWAVD-amide |
| Negative peptide 4 | Biotinyl-KKGFEVTTLADGTEVATSPL-amide |

The addition of the two Alanine residues to the amino terminus at this site was designed to increase immunogenicity and specificity. The addition of the charged -GKK residues at the carboxyterminus of the immunogen peptide was designed to overcome its increased hydrophobicity. In the event that the immunogen peptide formed micelles during the coupling reaction the presence of the charged GKK moiety adjacent to the Cysteine thiol would favour its accessibility to the BSA. The inclusion of mirror image KKG- at the aminoterminus of the reference peptide would favour the selection of antibody specific for the target sequence itself.

| | |
|---|---|
| Immunogen peptide | XA4P C-YLSALVSIRTDPS[pS]R-amide BSA conjugated |
| Reference peptide | Biotinyl-YLSALVSIRTDPS[PS]R-amide |
| Negative peptide 1 | Biotinyl-YLSAL VSIRTDPSSR-amide |
| Negative peptide 2 | Biotinyl-YLSALYSIRSDPA[PS]R-amide |
| Negative peptide 3 | Biotinyl-YLSALVSVRYDPS[pS]R-amide |
| Negative peptide 4 | Biotinyl-YLSAQIAIRTDPA[PS]R-amide |

All five A0X, A0XP, XA1, A4 and XA4P projects incorporating clonal selection for Reference peptide recognition by ELISA with limited or no binding to Negative Control peptides, followed by tissue and cell staining by selected clonal supernatants binding to tissues and cells, have been brought to successful conclusions. Affinity purified A0X, A0XP, XA1, A4 and XA4P monoclonals were obtained.

### Example 3: Uses of the Diagnostic Technology for the Detection and Characterisation of MAP Infections in Samples From Humans and Animals and in Food Safety

### 1. Detection and Measurement of MAP Infecting Human Gut Tissues

Endoscopic biopsies were studied from 45 people with Crohn's disease and some other disorders such as Irritable Bowel Syndrome (Scanu et al. Mycobacterium avium subspecies paratuberculosis infection in cases of Irritable Bowel Syndrome and comparison with Crohn's disease and Johne's disease: common neural and immune pathogenicities. J Clin Microbiol 2007: 45:3883-90). Samples were immediately fixed in formalin, followed by standard processing and embedding in paraffin Histopathology blocks. Preliminary work was carried out which identified 2 μm sections as optimal. Sections were treated with a standard antigen retrieval protocol. They were then stained with dilutions of monoclonal antibodies in the range 1 in 500 to 1 in 5000. Both direct fluorophore labelling of primary antibodies as well as the use of secondary antibodies labelled with fluorophore were employed. Tissues were stained with each of the primary antibodies A0X, A0XP, XA1, A4, XA4P used alone and viewed with using a Zeiss AxioSkop 2 microscope at magnifications of ×100 and ×200 to obtain a general impression of the distribution of MAP and then subsequently at ×400 and ×1000. MAP in humans is a Ziehl-Neelsen (Z-N) staining negative form and appears to be in the size range 0.3-1 μm. Higher magnification is required for satisfactory resolution.

A0X, XA1, A4 and XA4P all stained MAP in human gut, more specifically in endoscopic biopsies of the gut in all of the 45 people with Crohn's disease tested. However, staining of human gut tissues by A0XP was not seen in humans other than the occasional fluorescent signal from the lumen of a tissue blood vessel containing an A0XP positive cell in the blood. Unlike in animals, phosphorylation of A0X does not appear to occur widely in human gut. Phosphorylated A0XP however is seen in human blood in MAP infections. Staining of MAP by A0X, A0XP, XA1, A4 and XA4P is seen in human blood in MAP infection and in all people with Crohn's disease tested.

Antibodies were also used in combinations and viewed by confocal microscopy. Preferably antibodies were used in pairs. Preferred pairs were A0X with A0XP or XA1 from the amino terminus of the parent MAP molecule, and A4 with XA4P from the carboxyterminal end. Preferred pairs were also A0X with A4 and XA1 with A4 labelled with a red or green fluorophore respectively. This provided gold staining when the reagents colocalised specifically in the cytoplasm, not only of the same cells, but on the sub-micrometre MAP particles within the cytoplasm of infected cells. Such colocalisation provided strong confirmation of the specificity of MAP detection.

The use of antibody pairs comprising XA1 with A4 and A0X with A4 revealed a further aspect of the method. This is because whereas A0X and XA1 appeared to remain attached to the MAP organism itself or released to remain in the cell or displayed on the cell surface, A4 is frequently released from MAP to become displayed on the cell surface as well as released from the infected cell to traffic between cells. When A0X or XA1 are labelled with a red fluorophore and A4 labelled with a green one, the original gold colocalisation is progressively depleted to orange, and then to red as the green labelled A4 traffics to and enters other cells. Membrane bound structures filled with A4 green were seen, consistent with the presence of intercellular vesicles.

Biopsy samples from all the patients with Crohn's disease tested positive for MAP. This was observed in cells of the mucosal compartment, particularly the basal portion of the epithelial cells and the cytoplasm surrounding the basal portion of the mucus in goblet cells. Other MAP containing cells in the mucosal compartment were intra-epithelial macrophages and dendritic cells as well as intra-epithelial lymphocytes. MAP containing cells and free bacilli were also observed in the luminal mucus gel layer. In the lamina propria MAP infection was common in macrophages, polymorphs and B-lymphocytes. T-lymphocytes were rarely seen to be involved themselves, but frequently occurred adjacent to MAP filled macrophages. MAP positive cells were frequently seen in the lumen of small blood vessels. In duodenal biopsies MAP staining of Brunner's glands was limited only to the occasional macrophage filled with MAP while the glandular cells themselves were unaffected. However MAP containing cells were present in the interstitial connective tissue of Brunner's glands. A0XP in cells within tissues appeared to concentrate around the nucleus. These images of MAP in human tissues can be adapted to become quantitative and enable monitoring of the MAP infective load.

Surgical resection samples were also available from 4 patients with Crohn's disease. These samples permitted the examination not only the deeper layers of the gut through to the serosa, but also larger blood vessels, lymphatic vessels, extra intestinal fat wrapping and regional lymph nodes in the gut mesentery. As with the biopsy tissues the mucosa and sub-mucosa of each of these 4 patients were strongly positive for MAP. It was also found that the MAP infection extended right through the wall of the gut involving lymphatic vessels, the tissue between muscle layers and the serosa itself. In some sections lymphatic vessels full of stained MAP organisms were seen.

It has long been known that one of the pathological features of Crohn's disease is a vasculitis deemed to be autoimmune. The diagnostic antibodies showed that the thickened walls of such blood vessels were extensively infiltrated with MAP which also involved the surrounding perivascular connective tissues. Another characteristic pathological feature of Crohn's disease is the increase in fatty tissue around the gut. This is particularly well seen in the terminal ileum where it is termed ‘fat wrapping’. The adipocytes of this fat are known to be a rich source of the inflammatory marker CRP (C-reactive protein). The diagnostic method showed that the thin cytoplasm of the adipocytes in this tissue were extensively infected with MAP. Abundant MAP was also seen in cells of the interstitial connective tissues within the fat. MAP was also seen to involve regional lymph nodes.

The gut tissues of all 5 people diagnosed with Irritable Bowel Syndrome (IBS) who were tested were also seen to be widely infected with MAP in a manner very similar to CD. Positive MAP staining of gut endoscopic biopsies was also seen in cases of Thyroiditis and Psoriasis.

### 2. Animal Gut Tissues

Gut tissue samples were studied from 3 cows, 1 sheep, 1 goat, 1 red deer and 2 fallow deer all diagnosed with Johne's disease (JD). Autopsy samples were processed and stained with the primary antibodies, as described for humans. Tissues from all the animals were extensively infected with MAP which was generally present in the Ziehl-Neelsen positive phenotype. MAP in the guts of these animals diagnosed with Johne's disease stained with the A0X, A0XP, A4 and XA4P antibodies as well as with XA1. The microscopic appearance of MAP in animals was usually that of the classical Z-N positive mycobacterial phenotype but the monoclonal antibodies of the present work also demonstrated the pathogens in the paucimicrobial form.

The infective load of MAP in animals with Johne's disease was heavier than that found in humans in keeping with the well-recognised common pluribacillary form of JD. The MAP phenotype itself was consistent with ZN-positive cells. There was extensive involvement of mucosa and lamina propria and all layers of the gut. Cords of cells were seen which resulted from microvasculature full of MAP infected leucocytes. In addition, the thickened walls of vasculitic blood vessels and perivascular tissues were infiltrated with MAP infected cells as was seen in humans. MAP infection was also seen in neurovascular bundles affecting ganglion cells as well as nerve sheathes. This is consistent with the well described damage to the enteric nervous system of animals diagnosed with JD, much as in humans diagnosed with CD. A conspicuous difference between the gut tissues of these 5 ruminant species and humans is that A0XP is widely present in these animal gut tissues but appeared to be absent from human gut tissues.

### 3. Human Blood

Unlike in human gut tissues, A0XP, the phosphorylated form of A0X, is widely expressed in human blood. A0XP (MVINDDAQRLL[pS]QR) is the Serine phosphorylated form of A0X peptide in the extracellular amino terminal region of the IS900 protein. XA4P (YLSALVSIRTDPS[pS]R) is the A4 peptide in the extracellular carboxyterminal region of the IS900 protein with the distal of its two adjacent Serine residues phosphorylated. In CD both A0XP and XA4P are expressed within and on the surface of MAP-containing cells in human blood.

This provides 2 pairs of sterically accessible mutually exclusive antibodies on the P900 polypeptide for use in flow cytometry with A0X/A0XP on the amino terminal extracellular domain and A4/XA4P on the carboxy terminal extracellular domain. Each pair exists in a dynamic equilibrium as substrate and product, the sum of which provides a robust signal for determining the percentage of peripheral blood leucocyte populations infected with intra-cellular MAP. A0X/A0XP either remain attached to MAP or can be released within the cell and on the infected host cell's surface. A4/XA4P provide a similar signal but A4 can exit MAP infected cells and traffic between cells so that the cell populations containing A4/XA4P comprise those containing MAP organisms with an additional smaller population in which the A4/XA4P has been acquired by inter-cellular trafficking.

Flow cytometry on routine EDTA clinical blood samples was performed on 42 people with Crohn's disease using direct fluorophore labelled A0X-FITC/A0XP-PC5.5 and A4-FITC/XA4P-PC5.5 in an exploratory study. All people tested positive for MAP, with the proportion of the total circulating white blood cell population positive for MAP ranging from 3.9% to 47.1%. Use of phenotypic markers of the principal blood cell lineages enables a breakdown according to host cell type. These proportions were generally greater with A4/XA4P than they were with A0X/A0XP. The ratios A0X/A0XP and A4/XA4P provided a measure of phosphorylation activity. A high ratio of XA4P/A4 tended to characterise people with Crohn's disease in a higher state of activity. In such people intact monocytes in blood completely coated with segregated masses of A4/XA4P could be seen.

A second flow cytometry was carried out in 24 consecutive patients with Crohn's disease to determine the proportion (%) of total circulating white blood cells containing MAP.

Blood samples were collected into standard EDTA Vacutainer tubes. 100 μl of blood was then added to the required number of 12×75 mm round bottomed Falcon tubes. These were incubated for 5 minutes with 5 μl Human Seroblock (Bio-Rad). Anti-human CD45 APC conjugated antibody (Beckman Coulter) was added to all tubes to enable gating on the leucocyte populations. Half of the tubes (labelled ‘surface stained’) were then treated with the anti-MAP monoclonal antibodies for 15 minutes at room temperature in the dark. 0.5 mls OptiLyse C (Beckman Coulter) red blood cell lysis buffer was then added to all these tubes and incubated for 10 minutes at room temperature in the dark. The cells were then washed by adding 0.5 mls of PBS and centrifuged at 325 G for 5 minutes and the supernatant discarded.

All tubes were then fixed with 100 μl Fixation Medium A (Thermo Fisher Scientific) for 5 minutes then washed as above. The tubes that had not been stained with anti-MAP antibody (labelled ‘Permeabilised’) were incubated with Invitrogen Permeabilised Medium B (Thermo Fisher Scientific) to the pellet, anti-MAP monoclonal antibodies were added and incubated for 15 minutes at room temperature in the dark, followed by PBS washing as above. All tubes were then made up to 1 ml with flow buffer (PBS (Ca and Mg free), 0.2% sodium azide and 2% Bovine Serum Albumin (BSA). The samples were then acquired on a CytoFLEX flow cytometer (Beckman Coulter) gated on SSC vs CD45 and subsequently the data was an analysed using CytExpert software (Beckman Coulter).

The results of the second study in 24 people with Crohn's disease are shown in the table below (Table 2). There were 8 women and 16 men between the ages of 18 and 49 years. The numbers in the table along the rows indicate the % of the total circulating white blood cell population in each person stained by the corresponding MAP antibody A0X alone, A0XP alone or both A0X+A0XP as well as A4 alone, XA4P alone or both A4+XA4P. Because the A0 antibodies remain attached to their target peptides for longer than the A4 antibodies, the SUM of the A0 data in an individual person were taken as the measure of the % of circulating WBCs containing MAP (highlighted centre column). This SUM varies with the progress, clinical course and responses to treatment of Crohn's disease providing a direct access to the contribution to pathogenicity made by this unique multicopy insertion element.

The Flow Cytometry data from A4, XA4P and A4+XA4P provides a second direct insight into a probable contribution to pathogenicity of MAP by recording a further aspect of the in vivo function of P900. This is the ability to observe the phosphorylation and trafficking of the attached and released carboxy terminus both with the phosphorylation of the downstream serine as in the present work and that of the upstream partner and the presence and effect of dual phosphorylation.

Table 1 summarises the Flow Cytometry data obtained from 24 patients with Crohn's disease based on the use of the monoclonal antibodies A0X and A0XP on the extracellular amino-terminus of P900 on the left of the table and A4 and XA4P on the extracellular carboxy-terminus of P900 on the right. The data for each patient in each row separate into binding to the surface of white blood cells (surf) and binding to whole permeabilised cells (perm). The total proportion of cells infected by MAP is given by the sum of the percentages % in separate permeabilised cell populations identified by A0X alone, A0XP alone and A0X+A0XP (highlighted). This is the preferred measurement because A0X peptides tend to remain bound to the host intracellular mycobacterial cells longer than A4 peptides. On the right of the table are the results using A4, XA4P and A4+XA4P stained cell populations. In this study the strongly predicted phosphorylation of the distal serine in XA4P is used but similar studies may target the phosphorylated proximal serine of the pair or in dual phosphorylation.

The potential of the data comes together when we look at detail. The % total MAP loading across the group of 24 patients ranges from 1.52% to 48.9% and appears at the present stage to vary with the activity of the disease. Peaks or troughs in the % of MAP positive cells may follow the onset of anti-MAP treatments. More data will come as larger numbers of people are tested and with different diseases particularly in the “autoimmune and auto inflammatory” group, and with access to the loading of individual cell types. More data will also be obtained from studying the clinical correlates of phosphorylation events and monitoring the effects of different treatments.

The data show that the proportion of cells with A0X/A0XP or both on their surfaces is about half. In the permeabilised cells, the total cell percentage with A0X/A0XP is in close agreement with that using A4/XA4P, whereas the sum of A4/XA4P on the cell surface is considerably less than with A0X/A0XP. This would be consistent with a greater loss of A4/XA4P from the cell surface which is in keeping with its recognised greater mobility. Studies of the effect of these phosphorylation events will require larger clinical studies.

The flow cytometry system is the first example to be developed for paratuberculosis infection in Crohn's disease. This can now be used to study MAP infection in other diseases including especially psoriasis, thyroiditis, Parkinson's disease, type 1 diabetes, arthritis, ankylosing spondylitis, irritable bowel syndrome, ulcerative colitis, inflammatory bowel disease, Alzheimer's disease, multiple sclerosis, sarcoidosis, idiopathic pulmonary fibrosis and/or chronic fatigue syndrome. With Crohn's disease as with these other diseases, particularly “autoimmune conditions”, whether or not the presence of MAP is contributing to disease causation or progression will come from whether or not specific anti-MAP therapy leads to remission or healing of the disease. At present, the therapeutic T-cell vaccine against MAP is in early clinical trials.

TABLE 2

| | | | | | A0X + | A0X/ | | | A0X + | A0X/ |
|---|---|---|---|---|---|---|---|---|---|---|
| MAP Monoclonal | | | A0X | A0XP | A0XP | A0XP | A0X | A0XP | A0XP | A0XP |
| Patient | M/F | Age | surf | surf | surf | SUM | perm | perm | perm | SUM |
| 1 | F | 32 | 5.67 | 0.7 | 1.49 | 7.86 | 25.02 | 2.93 | 1.11 | 29.06 |
| 2 | M | 48 | 2.37 | 4.94 | 4.85 | 12.16 | 7.52 | 12.42 | 10.87 | 30.81 |
| 3 | M | 29 | 7.59 | 0.05 | 1.30 | 8.94 | 7.66 | 5.71 | 8.33 | 21.70 |
| 4 | M | 28 | 1.50 | 0.19 | 3.07 | 4.76 | 4.90 | 1.12 | 0.29 | 6.31 |
| 5 | M | 37 | 0.90 | 0.77 | 1.37 | 3.04 | 11.02 | 10.33 | 0.19 | 21.54 |
| 6 | F | 40 | 16.15 | 0.76 | 0.37 | 17.28 | 20.62 | 0.30 | 0.76 | 21.68 |
| 7 | F | 25 | 10.43 | 0.38 | 0.73 | 11.54 | 23.13 | 0.41 | 1.09 | 24.63 |
| 8 | M | 39 | 1.32 | 6.62 | 2.08 | 10.02 | 2.32 | 8.12 | 5.01 | 15.45 |
| 9 | M | 20 | 37.93 | 0.17 | 0.02 | 38.12 | 45.82 | 0.44 | 1.83 | 48.09 |
| 10 | M | 33 | 14.81 | 1.45 | 0.31 | 16.57 | 23.05 | 2.88 | 13.98 | 39.91 |
| 11 | F | 48 | 3.95 | 1.8 | 1.49 | 7.24 | 4.92 | 1.97 | 1.71 | 8.60 |
| 12 | F | 26 | 1.06 | 0.74 | 1.89 | 3.69 | 2.39 | 1.65 | 2.00 | 6.04 |
| 13 | M | 27 | 1.39 | 1.65 | 2.00 | 5.04 | 3.42 | 2.61 | 2.02 | 8.05 |
| 14 | M | 29 | 2.28 | 2.3 | 1.86 | 6.44 | 2.31 | 5.01 | 2.36 | 9.68 |
| 15 | M | 29 | 1.95 | 12.86 | 4.99 | 19.80 | 3.40 | 15.85 | 5.71 | 24.96 |
| 16 | M | 25 | 0.02 | 1.32 | 0.03 | 1.37 | 0.11 | 1.41 | 0.00 | 1.52 |
| 17 | M | 37 | 0.24 | 0.33 | 0.05 | 0.62 | 11.40 | 11.39 | 5.81 | 28.60 |
| 18 | M | 18 | 1.26 | 1.96 | 6.63 | 9.85 | 1.78 | 3.41 | 5.39 | 10.58 |
| 19 | F | 48 | 1.79 | 2.79 | 2.96 | 7.54 | 2.62 | 5.96 | 5.15 | 13.73 |
| 20 | F | 18 | 0.92 | 5.15 | 0.64 | 6.71 | 1.22 | 5.67 | 1.20 | 8.09 |
| 21 | M | 20 | 11.78 | 0.36 | 8.85 | 20.99 | 15.41 | 0.30 | 9.28 | 24.99 |
| 22 | M | 49 | 0.35 | 1.27 | 0.80 | 2.42 | 1.58 | 1.97 | 2.76 | 6.31 |
| 23 | M | 19 | 0.77 | 0.68 | 3.83 | 5.28 | 1.76 | 1.72 | 4.01 | 7.49 |
| 24 | F | 48 | 0.05 | 4.09 | 0.02 | 4.16 | 0.94 | 6.83 | 0.21 | 7.98 |
| SUM | | | | | | 231.44 | | | | 425.80 |
| average | | | | | | 9.64 | | | | 17.74 |
| | | | | | A4 + | A4/ | | | A4 + | A4/ |
| MAP Monoclonal | | | A4 | XA4P | XA4P | XA4P | A4 | XA4P | XA4P | XA4P |
| Patient | M/F | Age | surf | surf | surf | SUM | perm | perm | perm | SUM |
| 1 | F | 32 | 3.14 | 6.63 | 5.98 | 15.75 | 3.91 | 19.76 | 6.88 | 30.55 |
| 2 | M | 48 | 5.47 | 1.19 | 0.13 | 6.79 | 7.54 | 1.27 | 2.29 | 11.10 |
| 3 | M | 29 | 3.09 | 3.70 | 1.44 | 8.23 | 9.05 | 4.23 | 2.82 | 16.10 |
| 4 | M | 28 | 3.54 | 1.00 | 0.34 | 4.88 | 5.85 | 0.23 | 1.25 | 7.33 |
| 5 | M | 37 | 0.31 | 1.42 | 0.90 | 2.63 | 6.85 | 19.24 | 2.94 | 29.03 |
| 6 | F | 40 | 1.20 | 0.14 | 0.21 | 1.55 | 1.25 | 2.77 | 1.88 | 5.90 |
| 7 | F | 25 | 2.59 | 0.14 | 0.50 | 3.23 | 2.89 | 12.09 | 31.62 | 46.60 |
| 8 | M | 39 | 8.35 | 8.73 | 6.09 | 23.17 | 11.92 | 10.83 | 12.83 | 35.58 |
| 9 | M | 20 | 0.41 | 0.00 | 0.12 | 2.50 | 19.89 | 5.76 | 3.01 | 28.66 |
| 10 | M | 33 | 21.26 | 0.03 | 1.23 | 22.52 | 7.90 | 0.84 | 41.03 | 49.77 |
| 11 | F | 48 | 2.20 | 0.02 | 0.28 | 2.50 | 2.35 | 4.17 | 1.72 | 8.24 |
| 12 | F | 26 | 0.45 | 0.15 | 0.10 | 0.70 | 2.77 | 0.96 | 0.81 | 4.54 |
| 13 | M | 27 | 2.17 | 0.96 | 0.81 | 3.94 | 3.04 | 1.46 | 1.35 | 5.85 |
| 14 | M | 29 | 0.30 | 6.31 | 2.50 | 9.11 | 2.33 | 11.74 | 7.91 | 21.98 |
| 15 | M | 29 | 0.00 | 0.56 | 2.45 | 3.01 | 0.00 | 6.98 | 8.26 | 15.24 |
| 16 | M | 25 | 0.68 | 0.64 | 0.46 | 1.78 | 1.38 | 1.37 | 0.47 | 3.22 |
| 17 | M | 37 | 0.05 | 2.24 | 0.14 | 2.43 | 2.27 | 4.31 | 1.09 | 7.67 |
| 18 | M | 18 | 0.36 | 9.11 | 1.53 | 11.00 | 0.42 | 13.6 | 1.76 | 15.78 |
| 19 | F | 48 | 1.04 | 3.40 | 3.60 | 8.04 | 2.43 | 4.88 | 8.14 | 15.45 |
| 20 | F | 18 | 5.12 | 0.53 | 2.61 | 8.26 | 5.62 | 0.85 | 5.61 | 12.08 |
| 21 | M | 20 | 0.85 | 2.70 | 0.36 | 3.91 | 19.92 | 4.1 | 6.84 | 30.86 |
| 22 | M | 49 | 8.80 | 0.31 | 7.08 | 16.19 | 9.06 | 0.58 | 9.12 | 18.76 |
| 23 | M | 19 | 0.38 | 0.21 | 0.21 | 0.80 | 2.06 | 0.76 | 0.41 | 3.23 |
| 24 | F | 48 | 0.00 | 6.76 | 4.62 | 11.38 | 0.49 | 7.81 | 4.91 | 13.21 |
| SUM | | | | | | 174.30 | | | | 436.73 |
| average | | | | | | 7.26 | | | | 18.20 |

### 3.2 Cytology

Cells isolated from peripheral blood were stained with a combination of two directly conjugated monoclonal antibodies: A0X (FITC/Green)+A0XP (Cy 5.5/Red) or A4 FITC/Green)+XA4P (Cy 5.5/Red). Confocal images were viewed using a Leica SP-2 confocal microscope, recorded and stored in JPEG format.

Results: peripheral blood cells showed considerable heterogeneity in their staining pattern with cells either negative, positive for a single antibody only, or positive for both antibodies. This latter observation is demonstrated by the clear co-localisation of the fluorescent reporter molecules. Although the phenotype of positive cells is yet to be established DIC (differential interference contrast) imaging and Flow cytometry data indicate that positive cells are non-lymphocytic in origin.

### 4. Animal Blood

### 4.1 Cats

A domestic cat (Cat 1) became unwell with weight loss, diarrhoea, distended abdomen and poor general condition. Ultrasound scan of the abdomen of the clinically affected animal showed thickening of the wall throughout the colon. Endoscopy and biopsy by the veterinarian showed clinically and histologically that the animal had Inflammatory Bowel Disease. Flow cytometry was performed on 2 EDTA blood samples over a period of 4 months. The proportion of total circulating white blood cells infected with MAP in the cat was 7.6% and 9.8%. Immunofluorescence microscopy on the endoscopic biopsy samples from Cat 1 confirmed the presence of MAP with a histological appearance similar to that seen in both animals with Johne's disease and humans with Crohn's disease.

During this period, a new kitten (Cat 2) was introduced to the same household. It was clinically well at the time of purchase from the breeder. A week following introduction to the household, the kitten developed bloody diarrhoea. Routine stool microbiology was negative. Flow cytometry was again performed on 2 EDTA blood samples over a period of 4 months. The proportion of circulating white cells infected with MAP was 16.4% and 14.3%. These data confirmed that both animals had a systemic MAP infection.

### 4.2 Dairy Cows

EDTA blood samples were obtained from 4 dairy cows. These animals were part of a closed dairy herd of more than 20 years standing with no known clinical cases of Johne's disease. Intermittent ELISA testing of individual milk samples from the herd had shown that one of the 4 sampled cows had had 2 positive ELISA readings and 2 other sampled cows had had 1 positive ELISA reading amongst multiple negative results. The fourth cow had had no raised ELISA readings on milk at all. Flow cytometry was performed on the 4 blood samples using A0X/A0XP and A4/XA4P pairs of monoclonal antibodies. The results showed that the proportions of the total circulating white blood cell populations in these animals infected with intracellular MAP, were 10.9%, 36.3%, 40.1%, and 45.2%. These results are a further indication of the ability of a significant systemic MAP infection to persist in a subclinical state. They also demonstrate that the present diagnostic technology has a much greater sensitivity than conventional diagnostic methods with the ability to reveal the true scale of the long term threat to animal and human health posed by these pathogens.

### 5. Human Breast Milk

A 3 month old male child presented with rectal bleeding and episodes of abdominal pain. He was investigated including upper and lower Gastrointestinal endoscopy with multiple biopsies, which led to the establishment of a diagnosis of Crohn's disease at 8 months. MAP tests, subsequently requested and carried out on his paraffin embedded histopathology blocks showed extensive infection with MAP of the stomach and duodenum and in all biopsies from the terminal ileum to the rectum. His mother, who did not have Crohn's disease, had never fed him anything except her own milk. However she had been diagnosed with auto-immune thyroiditis which is linked genetically to Crohn's disease. MAP testing requested by her on a 50 ml sample of expressed breast milk showed abundant MAP infected cells in the centrifugal pellet.

### 6. Human Skin Samples in Psoriasis

3-4 mm punch biopsy full thickness skin samples were obtained under local anaesthesia from 2 adults each diagnosed with Psoriasis. Samples were taken from the central region of a psoriatic skin lesion and an additional sample from the periphery of the lesion overlapping with normal skin. A normal skin sample between lesions was also obtained. Samples were formalin fixed, processed and embedded in routine histopathology blocks, following standard procedures. Sections of 2 μm were cut, immobilised on Vectabond microscope slides, treated for antigen retrieval, and stained with XA1/A4 monoclonal antibodies and examined by confocal microscopy.

Biopsies taken from within the psoriatic lesions were positive for MAP in both adults. Gold colocalisation of XA1 and A4 was seen in inflammatory cells in the thickened epidermal layer with staining persisting into the stratum corneum. Staining was also conspicuous in the germinal layer. Positive MAP staining extended throughout inflammatory cells in the rete and in inflammatory cells within the dermis. MAP positive cells were also seen around the hair follicles. A conspicuous associated feature was the presence of MAP within the pilo-sebaceous unit.

An abnormality of sebaceous glands might contribute to the dry, scaly nature of the superficial layers of psoriatic plaques. A further conspicuous feature in the dermis was MAP involvement of neurovascular bundles with colocalising XA1/A4 staining of these pathogens within thickened arterial walls and perivascular connective tissues. MAP staining of adjacent nerve bundles was also seen. Staining from biopsies taken at the periphery of psoriatic plaques showed that MAP staining stopped at the boundary between the plaque and normal skin. MAP was also absent from biopsies of normal looking skin between plaques. This would be consistent with a role for MAP in psoriatic plaque formation.

### 7. Measuring the Proportion of MAP Positive Cells in Synovial Joint Fluid in Arthritis

An adult female human with Psoriasis presented with discomfort and an acute effusion in her right knee joint. There was no history of trauma. The joint was warm and distended but was not acutely tender. Other joints were unaffected. A 20 ml sample of straw coloured slightly opalescent fluid was aspirated and the cells separated by centrifugation. These were washed, stained with fluorophore labelled A0X/A0XP and examined by flow cytometry. The proportion of cells containing MAP in the joint fluid was 8.56%. This was similar to the % of MAP-positive peripheral white blood cells in her blood at the time.

### Example 4: Construction of New ChAdOx2 Vaccines

Based on the above results, the hAd5 HAV vaccine described in Example 1 has been developed to include additional MAP peptides at the amino terminus. Two new vaccines have been produced: HAVX1 and HAVX2. HAVX1 and HAVX2 are five gene vaccines, whilst HAV (Bull et al. 2007 PLoS ONE 2(11): e1229; Bull et al. 2014 Veterinary Research 45:112; Example 1 above) is a four gene vaccine. HAVX1 encodes the sequence:

MTVTEVVVAQPVWAGVDAGKADHYCMVINDDAQRLLSQRVANDEAALLEL
IAAVTTLADGGEVTWAIDLNAGGAALLIALLIAAGQRLLYIPGATVHHAA
GSYRGE

followed by the 2A sequence (APVKQTLNFDLLKLAGDVESNPGP)—followed by HAV (SEQ ID NO: 41).

The HAVX1 represents the normal extra-mycobacterial region, the transmembrane region and the first intra-mycobacterial portion of the P900 sequence stopping short of the active site mechanism of the putative transposase. It has no known toxicity and is abundantly expressed in vivo.

HAVX2 encodes the sequence:

MVINDDAQRLLSQRVDAGKADHYAVTTLADGGEVTWAIDLNAGGAALLIA
LLIAAGQRLLYIPGATVHHAAGSYRGE

followed by the 2A sequence (APVKQTLNFDLLKLAGDVESNPGP)—followed by HAV (SEQ ID NO: 41).

The HAVX2 disrupts the normal sequence, places emphasis on the N-terminal epitope and exposes a further T-cell epitope just before it dips into the transmembrane sequence.

These rearrangements are not predicted to introduce any risk and are compatible with the safe handling of new ChAdOx2 HAVX1 and ChAdOx2 HAVX2 vaccine constructs.

1. A Mycobacterium avium subspecies paratuberculosis (MAP) vaccine comprising a polynucleotide encoding a polypeptide which comprises a fragment of MAP P900, wherein the fragment of MAP P900 has a length of up to 150 amino acids and comprises an amino acid sequence of at least 9 contiguous amino acids from the region of MAP P900 shown in SEQ ID NO: 2.

2. The MAP vaccine of claim 1, wherein the polypeptide comprises the amino acid sequence MVINDDAQRLLSQR (SEQ ID NO: 3).

3. The MAP vaccine of claim 2, wherein the polypeptide comprises the amino acid sequence
(SEQ ID NO: 4)
MVINDDAQRLLSQRVANDEAALLELI

4. (canceled)

5. The MAP vaccine of claim 1, wherein the polypeptide comprises the amino acid sequence VTTLADGGEVTWAID (SEQ ID NO: 7).

6. The MAP vaccine of claim 5, wherein the polypeptide comprises the amino acid sequence VTTLADGGEVTWAIDLNA (SEQ ID NO: 8).

7. The MAP vaccine of claim 1, wherein the polypeptide comprises the amino acid sequence EVVVAQPVWAGVDAGKADHY (SEQ ID NO: 9).

8. The MAP vaccine of claim 7, wherein the polypeptide comprises the amino acid sequence
(SEQ ID NO: 10)
MTVTEVVVAQPVWAGVDAGKADHY

9. The MAP vaccine of claim 1, which comprises one or more polynucleotides encoding two or more polypeptides, each comprising a fragment of MAP P900, wherein the fragment of MAP P900 has a length of up to 150 amino acids and comprises an amino acid sequence of at least 9 contiguous amino acids from the region of MAP P900 shown in SEQ ID NO: 2.

10. (canceled)

11. The MAP vaccine of claim 1, which comprises one or more polynucleotides encoding at least two of the following polypeptides: a polypeptide comprising the amino acid sequence MVINDDAQRLLSQR (SEQ ID NO: 3), a polypeptide comprising the amino acid sequence VTTLADGGEVTWAID (SEQ ID NO: 7), and a polypeptide comprising the amino acid sequence
(SEQ ID NO: 9)
EVVVAQPVWAGVDAGKADHY

12. The MAP vaccine of claim 11, wherein the one or more polynucleotides encode a polypeptide comprising the amino acid sequence MVINDDAQRLLSQRVANDEAALLELI (SEQ ID NO: 4), a polypeptide comprising the amino acid sequence VTTLADGGEVTWAIDLNA (SEQ ID NO: 8) and/or a polypeptide comprising the amino acid sequence MTVTEVVVAQPVWAGVDAGKADHY (SEQ ID NO: 10).

13. The MAP vaccine of claim 1, wherein the polypeptide comprises the amino acid sequence:
(i)
(SEQ ID NO: 11)
MVINDDAQRLLSQRX₂VTTLADGGEVTWAID;
(ii)
(SEQ ID NO: 12)
MVINDDAQRLLSQRX₂VTTLADGGEVTWAIDLNA;
(iii)
(SEQ ID NO: 13)
MVINDDAQRLLSQRVANDEAALLELIXVTTLADGGEVTWAID;
(iii)
(SEQ ID NO: 14)
MVINDDAQRLLSQRVANDEAALLELIX₂VTTLADGGEVTWAIDLNA;
(iv)
(SEQ ID NO: 15)
EVVVAQPVWAGVDAGKADHYX₁MVINDDAQRLLSQR;
(vi)
(SEQ ID NO: 16)
EVVVAQPVWAGVDAGKADHYX₁MVINDDAQRLLSQRVANDEAALLELI;
(vii)
(SEQ ID NO: 17)
MTVTEVVVAQPVWAGVDAGKADHYX₁MVINDDAQRLLSQR;
(viii)
(SEQ ID NO: 18)
MTVTEVVVAQPVWAGVDAGKADHYX₁MVINDDAQRLLSQRVANDEAALLE
LI;
(ix)
(SEQ ID NO: 52)
MVINDDAQRLLSQRVDAGKADHYAVTTLADGGEVTWAID;
(x)
(SEQ ID NO: 146)
MVINDDAQRLLSQRVDAGKADHYAVTTLADGGEVTWAIDLNA;
(xi)
(SEQ ID NO: 60)
MVINDDAQRLLSQRVDAGKADHYAVTTLADGGEVTWAIDLNAGGAALLIA
LLIAAGQRLLYIPG[[X₃]]ATVHHAAGSYRGE;
[[or]]
(xii)
(SEQ ID NO: 61)
MVINDDAQRLLSQRVDAGKADHYAVTTLADGGEVTWAIDLNAGGAALLIA
LLIAAGQRLLYIPGRTVHHAAGSYRGE;
(xiii)
(SEQ ID NO: 83)
MTVTEVVVAQPVWAGVDAGKADHYCMVINDDAQRLLSQRVANDEAALLEL
IAAVTTLADGGEVTWAIDLNAGGAALLIALLIAAGQRLLYIPG[[X₃]]
ATVHHAAGSYRGE;
or
(xiv)
(SEQ ID NO: 58)
MTVTEVVVAQPVWAGVDAGKADHYCMVINDDAQRLLSQRVANDEAALLEL
IAAVTTLADGGEVTWAIDLNAGGAALLIALLIAAGQRLLYIPGRTVHHAA
GSYRGE,
wherein X₁ is a peptide linker or C, X₂ is a peptide linker, A or AA.

14. The MAP vaccine of claim 13, wherein the polypeptide comprises the amino acid sequence
(i)
(SEQ ID NO: 19)
EVVVAQPVWAGVDAGKADHYXIMVINDDAQRLLSQRVANDE
AALLELI X₂VTTLADGGEVTWAIDLNA;
or
(ii)
(SEQ ID NO: 20)
MTVTEVVVAQPVWAGVDAGKADHYXIMVINDDAQRLLSQRV
ANDEAALLELI X₂VTTLADGGEVTWAIDLNA,
wherein X₁ is a peptide linker or C and X₂ is a peptide linker, A or AA.

15. (canceled)

16. The MAP vaccine of claim 1, further comprising at least one polynucleotide encoding an additional MAP polypeptide, or a fragment thereof.

17-18. (canceled)

19. The MAP vaccine of claim 17, wherein the additional MAP polypeptide is at least one of the following: a ahpC polypeptide, a gsd polypeptide, a p12 polypeptide and a mpa polypeptide, wherein:
(a) said ahpC polypeptide has the amino acid sequence given in SEQ ID NO: 32; and/or
(b) said gsd polypeptide has the amino acid sequence given in SEQ ID NO: 34; and/or
(c) said pl2 polypeptide has the amino acid sequence given in SEQ ID NO: 36 or SEQ ID NO: 38; and/or
(d) said mpa polypeptide bas the amino acid sequence given in SEQ ID NO: 40.

20. The MAP vaccine of claim 19, wherein the polypeptide comprises the amino acid sequence shown in SEQ ID NO: 41, wherein a peptide comprising the amino acid sequence MVINDDAQRLLSQR is added at the N-terminus or inserted between: positions 7 and 8, positions 199 and 200, positions 442 and 443, positions 577 and 578, and/or positions 820 and 821; or the amino acid sequence shown in SEQ ID NO: 42, wherein a peptide comprising the amino acid sequence MVINDDAQRLLSQR is added at the N-terminus or inserted between: positions 7 and 8, positions 199 and 200, positions 442 and 443, positions 582 and 583, and/or positions 825 and 826.

21. The MAP vaccine of claim 20, wherein the polypeptide comprises the amino acid sequence shown in any one of SEQ ID NOs: 62, 63, 64, 65, 67, 68 and 69.

22-36. (canceled)

37. The MAP vaccine of claim 1, wherein the polynucleotide is comprised in a vector.

38. (canceled)

39. The MAP vaccine claim 37, wherein the vector is a viral vector.

40. The MAP vaccine of claim 39, wherein the viral vector is an adenoviral vector or a poxvirus vector.

41. (canceled)

42. A method of treating or preventing MAP infection or a condition or symptom associated with MAP infection comprising administering to a subject in need thereof an effective amount of the MAP vaccine of claim 1.

43. (canceled)
